SEQUENCE LISTING (93 sequences)

SEQ ID NO: 1 - protein, 856 aa, Homo sapiens
MKPRPAGFVDNKLKQRVIQYLTSNKCGKYVDIGVLASDLQRVYSIDYGRRKRNAFRIQVEKVFSIISSEKELKNLTELEDEHLAKRARQGEEDNEYTESYSDDDSSMEDYPDPQSANHMNSSLLSLYRKGNPDSVSNTPEMEQRETTSSTPRISSKTGSIPLKTPAKDSEGGWFIDKTPSVKKDSFFLDLSCEKSNPKKPITEIQDSKDSSLLESDMKRKGKLKNKGSKRKKEDLQEVDGEIEAVLQKKAKARGLEFQISNVKFEDVGGNDMTLKEVCKMLIHMRHPEVYHHLGVVPPRGVLLHGPPGCGKTLLAHAIAGELDLPILKVAAPEIVSGVSGESEQKLRELFEQAVSNAPCIIFIDEIDAITPKREVASKDMERRIVAQLLTCMDDLNNVAATARVLVIGATNRPDSLDPALRRAGRFDREICLGIPDEASRERILQTLCRKLRLPQAFDFCHLAHLTPGFVGADLMALCREAAMCAVNRVLMKLQEQQKKNPEMEDLPSKGVQEERLGTEPTSETQDELQRLLGLLRDQDPLSEEQMQGLCIELNDFIVALSSVQPSAKREGFVTVPNVTWADIGALEDIREELTMAILAPVRNPDQFKALGLVTPAGVLLAGPPGCGKTLLAKAVANESGLNFISVKGPELLNMYVGESERAVRQVFQRAKNSAPCVIFFDEVDALCPRRSDRETGASVRVVNQLLTEMDGLEARQQVFIMAATNRPDIIDPAILRPGRLDKTLFVGLPPPADRLAILKTITKNGTKPPLDADVNLEAIAGDLRCDCYTGADLSALVREASICALRQEMARQKSGNEKGELKVSHKHFEEAFKKVRSSISKKDQIMYERLQESLSR

SEQ ID NO: 2 - protein, 659 aa, Homo sapiens
MEDYPDPQDSKDSSLLESDMKRKGKLKNKGSKRKKEDLQEVDGEIEAVLQKKAKARGLEFQISNVKFEDVGGNDMTLKEVCKMLIHMRHPEVYHHLGVVPPRGVLLHGPPGCGKTLLAHAIAGELDLPILKVAAPEIVSGVSGESEQKLRELFEQAVSNAPCIIFIDEIDAITPKREVASKDMERRIVAQLLTCMDDLNNVAATARVLVIGATNRPDSLDPALRRAGRFDREICLGIPDEASRERILQTLCRKLRLPQAFDFCHLAHLTPGFVGADLMALCREAAMCAVNRVLMKLQEQQKKNPEMEDLPSKGVQEERLGTEPTSETQDELQRLLGLLRDQDPLSEEQMQGLCIELNDFIVALSSVQPSAKREGFVTVPNVTWADIGALEDIREELTMAILAPVRNPDQFKALGLVTPAGVLLAGPPGCGKTLLAKAVANESGLNFISVKGPELLNMYVGESERAVRQVFQRAKNSAPCVIFFDEVDALCPRRSDRETGASVRVVNQLLTEMDGLEARQQVFIMAATNRPDIIDPAILRPGRLDKTLFVGLPPPADRLAILKTITKNGTKPPLDADVNLEAIAGDLRCDCYTGADLSALVREASICALRQEMARQKSGNEKGELKVSHKHFEEAFKKVRSSISKKDQIMYERLQESLSR

SEQ ID NO: 3 - DNA, 638 bp, Artificial Sequence
  NVL gene synthesis
agcggtctcacatgaagccacgtcctgcaggatttgtggacaacaaactcaagcaacgcgttattcagtatctgaccagtaacaagtgcggcaaatacgtagacataggggttttggcgtctgatctgcagagggtgtactctatcgactatggtcggaggaaacggaatgcctttcgaattcaggtcgaaaaggtgttctccatcatcagctcagagaaagagctgaagaatctcactgaacttgaggacgaacatctcgccaaaagagccagacaaggcgaagaggacaatgagtacacagagtcctacagtgatgatgactcatcaatggaggactatcctgatccccagtcagctaaccacatgaactcttctctgcttagcctgtataggaaggggaatcccgatagtgtgagcaacacaccggaaatggagcagagagaaaccaccagttccactccacgcatatcctccaaaaccggtagcattcccttgaaaactcctgctaaagactctgaaggaggctggtttatcgacaaaacgcctagcgtcaagaaggatagcttcttcctggatctgagctgtgagaagtccaatcccaagaagccaatcacagagattcaggatgagaccgaa

SEQ ID NO: 4 - DNA, 29 bp, Artificial Sequence
  sense NVL-IF1
ataggtctccaggattcaaaagattcttc

SEQ ID NO: 5 - DNA, 34 bp, Artificial Sequence
  asense NVL-IF1
tatggtctcatcgacccggctgagggactcctgc

SEQ ID NO: 6 - DNA, 1980 bp, Artificial Sequence
  NVL-PCR-fragment
ataggtctccaggattcaaaagattcttctcttttggagagtgatatgaaacggaaaggcaagctaaagaataaaggaagcaaaaggaagaaagaagatcttcaggaagtagatggagaaattgaagctgtcctacaaaagaaagctaaagccagggggttagaattccagatctccaacgtgaagtttgaagatgtgggaggcaatgatatgacattaaaagaggtctgcaagatgctcatacacatgcgtcacccggaggtgtaccaccacctgggcgtcgtgccccctcgtggagttctccttcatggaccaccaggctgtgggaagacattacttgcacatgcaattgctggggaacttgacctgccaattttgaaagtggctgctccagagattgtgtctggagtatccggagagtctgagcagaagctgagagaactatttgagcaagctgtgtcaaatgcaccatgtatcattttcattgatgaaattgatgctattacccccaaaagagaagtggcttcaaaagatatggaacgaagaattgtagcccaactcctaacctgcatggatgatctgaataatgtggctgctacagcccgggtcctagttattggagctactaatcgaccagactcgttagaccctgctttgagacgtgcgggaaggttcgaccgagaaatatgcctaggtatcccagatgaagcatccagggaaagaatacttcaaacattgtgcagaaaactgaggcttcctcaagcttttgatttctgtcacttagcacacctaactccaggctttgttggtgctgatctcatggcactgtgccgagaggcagcaatgtgtgcagtcaatagagtcttaatgaagctacaggaacagcagaagaaaaatcctgaaatggaagatttgccatctaaaggagtccaggaggaaaggctgggaactgagcccacttctgaaacacaggatgaattacaaaggctgctggggttgctaagagaccaagatcccctctcagaggagcagatgcaaggactgtgcattgaactgaatgatttcattgttgctctatcctcagtccaaccctctgccaaaagggaaggctttgtcactgtccctaatgtgacatgggcagatattggtgccctggaagacattagagaggagctcaccatggcaatattggcaccagtacgcaacccagaccagttcaaagctcttggattggtgactccagctggggtcctccttgctggtcctcctggctgtgggaagactctgctggcgaaggctgttgcaaatgagtccggactaaattttatatctgtcaagggccccgaattactaaacatgtatgttggtgagagtgaacgtgctgtgcgacaagtttttcaacgagccaagaactcagcaccctgtgtgatattctttgatgaagtggatgctttatgtcctcgaagatcagaccgagagacaggggcaagtgtccgagtggtgaatcagctacttacagagatggatggtctggaagcacgccagcaggtttttattatggcagccactaacaggccagatataattgaccctgcaatcctgcgcccgggccgcctggacaaaacactgtttgtgggtttaccgccccctgcagatcgccttgccatcttaaaaactatcacaaaaaatggtaccaaaccaccactggatgcagatgtaaatttggaagcaattgctggtgaccttcgctgtgattgctatacgggcgcagatctctctgctttggtacgagaagcttctatctgtgccctgagacaggaaatggcaagacagaagagtggaaatgaaaaaggtgaactcaaggttagtcataagcattttgaagaagctttcaagaaagtaagatcatctatatcaaaaaaggatcaaatcatgtatgaacgtttgcaggagtccctcagccgggtcgatgagaccata

SEQ ID NO: 7 - DNA, 8182 bp, Artificial Sequence
  pTriEx-1-NVL[human]-IF1-H8
ggagtcgctgcgacgctgccttcgccccgtgccccgctccgccgccgcctcgcgccgcccgccccggctctgactgaccgcgttactcccacaggtgagcgggcgggacggcccttctccttcgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcggaccgaaattaatacgactcactataggggaattgtgagcggataacaattccccggagttaatccgggacctttaattcaacccaacacaatatattatagttaaataagaattattatcaaatcatttgtatattaattaaaatactatactgtaaattacattttatttacaatcaaaggagatataccatgaagccacgtcctgcaggatttgtggacaacaaactcaagcaacgcgttattcagtatctgaccagtaacaagtgcggcaaatacgtagacataggggttttggcgtctgatctgcagagggtgtactctatcgactatggtcggaggaaacggaatgcctttcgaattcaggtcgaaaaggtgttctccatcatcagctcagagaaagagctgaagaatctcactgaacttgaggacgaacatctcgccaaaagagccagacaaggcgaagaggacaatgagtacacagagtcctacagtgatgatgactcatcaatggaggactatcctgatccccagtcagctaaccacatgaactcttctctgcttagcctgtataggaaggggaatcccgatagtgtgagcaacacaccggaaatggagcagagagaaaccaccagttccactccacgcatatcctccaaaaccggtagcattcccttgaaaactcctgctaaagactctgaaggaggctggtttatcgacaaaacgcctagcgtcaagaaggatagcttcttcctggatctgagctgtgagaagtccaatcccaagaagccaatcacagagattcaggattcaaaagattcttctcttttggagagtgatatgaaacggaaaggcaagctaaagaataaaggaagcaaaaggaagaaagaagatcttcaggaagtagatggagaaattgaagctgtcctacaaaagaaagctaaagccagggggttagaattccagatctccaacgtgaagtttgaagatgtgggaggcaatgatatgacattaaaagaggtctgcaagatgctcatacacatgcgtcacccggaggtgtaccaccacctgggcgtcgtgccccctcgtggagttctccttcatggaccaccaggctgtgggaagacattacttgcacatgcaattgctggggaacttgacctgccaattttgaaagtggctgctccagagattgtgtctggagtatccggagagtctgagcagaagctgagagaactatttgagcaagctgtgtcaaatgcaccatgtatcattttcattgatgaaattgatgctattacccccaaaagagaagtggcttcaaaagatatggaacgaagaattgtagcccaactcctaacctgcatggatgatctgaataatgtggctgctacagcccgggtcctagttattggagctactaatcgaccagactcgttagaccctgctttgagacgtgcgggaaggttcgaccgagaaatatgcctaggtatcccagatgaagcatccagggaaagaatacttcaaacattgtgcagaaaactgaggcttcctcaagcttttgatttctgtcacttagcacacctaactccaggctttgttggtgctgatctcatggcactgtgccgagaggcagcaatgtgtgcagtcaatagagtcttaatgaagctacaggaacagcagaagaaaaatcctgaaatggaagatttgccatctaaaggagtccaggaggaaaggctgggaactgagcccacttctgaaacacaggatgaattacaaaggctgctggggttgctaagagaccaagatcccctctcagaggagcagatgcaaggactgtgcattgaactgaatgatttcattgttgctctatcctcagtccaaccctctgccaaaagggaaggctttgtcactgtccctaatgtgacatgggcagatattggtgccctggaagacattagagaggagctcaccatggcaatattggcaccagtacgcaacccagaccagttcaaagctcttggattggtgactccagctggggtcctccttgctggtcctcctggctgtgggaagactctgctggcgaaggctgttgcaaatgagtccggactaaattttatatctgtcaagggccccgaattactaaacatgtatgttggtgagagtgaacgtgctgtgcgacaagtttttcaacgagccaagaactcagcaccctgtgtgatattctttgatgaagtggatgctttatgtcctcgaagatcagaccgagagacaggggcaagtgtccgagtggtgaatcagctacttacagagatggatggtctggaagcacgccagcaggtttttattatggcagccactaacaggccagatataattgaccctgcaatcctgcgcccgggccgcctggacaaaacactgtttgtgggtttaccgccccctgcagatcgccttgccatcttaaaaactatcacaaaaaatggtaccaaaccaccactggatgcagatgtaaatttggaagcaattgctggtgaccttcgctgtgattgctatacgggcgcagatctctctgctttggtacgagaagcttctatctgtgccctgagacaggaaatggcaagacagaagagtggaaatgaaaaaggtgaactcaaggttagtcataagcattttgaagaagctttcaagaaagtaagatcatctatatcaaaaaaggatcaaatcatgtatgaacgtttgcaggagtccctcagccgggtcgagcaccaccatcaccatcaccatcactaagtgattaacctcaggtgcaggctgcctatcagaaggtggtggctggtgtggccaatgccctggctcacaaataccactgagatcgatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgtaactagcataaccccttggggcctctaaacgggtcttgaggggttttttgctgaaagcatgcggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaatagagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggaacgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgtccgcgcgtttcctgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgtcaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcttcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgcatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 8 - protein, 866 aa, Artificial Sequence
  NVL[human]-IF1-H8
MKPRPAGFVDNKLKQRVIQYLTSNKCGKYVDIGVLASDLQRVYSIDYGRRKRNAFRIQVEKVFSIISSEKELKNLTELEDEHLAKRARQGEEDNEYTESYSDDDSSMEDYPDPQSANHMNSSLLSLYRKGNPDSVSNTPEMEQRETTSSTPRISSKTGSIPLKTPAKDSEGGWFIDKTPSVKKDSFFLDLSCEKSNPKKPITEIQDSKDSSLLESDMKRKGKLKNKGSKRKKEDLQEVDGEIEAVLQKKAKARGLEFQISNVKFEDVGGNDMTLKEVCKMLIHMRHPEVYHHLGVVPPRGVLLHGPPGCGKTLLAHAIAGELDLPILKVAAPEIVSGVSGESEQKLRELFEQAVSNAPCIIFIDEIDAITPKREVASKDMERRIVAQLLTCMDDLNNVAATARVLVIGATNRPDSLDPALRRAGRFDREICLGIPDEASRERILQTLCRKLRLPQAFDFCHLAHLTPGFVGADLMALCREAAMCAVNRVLMKLQEQQKKNPEMEDLPSKGVQEERLGTEPTSETQDELQRLLGLLRDQDPLSEEQMQGLCIELNDFIVALSSVQPSAKREGFVTVPNVTWADIGALEDIREELTMAILAPVRNPDQFKALGLVTPAGVLLAGPPGCGKTLLAKAVANESGLNFISVKGPELLNMYVGESERAVRQVFQRAKNSAPCVIFFDEVDALCPRRSDRETGASVRVVNQLLTEMDGLEARQQVFIMAATNRPDIIDPAILRPGRLDKTLFVGLPPPADRLAILKTITKNGTKPPLDADVNLEAIAGDLRCDCYTGADLSALVREASICALRQEMARQKSGNEKGELKVSHKHFEEAFKKVRSSISKKDQIMYERLQESLSRVEHHHHHHHH

SEQ ID NO: 9 - DNA, 7800 bp, Artificial Sequence
  pET24d-NVL[human]-IF1-H6
ggggaattgtgagcggataacaattcccctctagaaataattttgtttaactttaagaaggagatataccatgaagccacgtcctgcaggatttgtggacaacaaactcaagcaacgcgttattcagtatctgaccagtaacaagtgcggcaaatacgtagacataggggttttggcgtctgatctgcagagggtgtactctatcgactatggtcggaggaaacggaatgcctttcgaattcaggtcgaaaaggtgttctccatcatcagctcagagaaagagctgaagaatctcactgaacttgaggacgaacatctcgccaaaagagccagacaaggcgaagaggacaatgagtacacagagtcctacagtgatgatgactcatcaatggaggactatcctgatccccagtcagctaaccacatgaactcttctctgcttagcctgtataggaaggggaatcccgatagtgtgagcaacacaccggaaatggagcagagagaaaccaccagttccactccacgcatatcctccaaaaccggtagcattcccttgaaaactcctgctaaagactctgaaggaggctggtttatcgacaaaacgcctagcgtcaagaaggatagcttcttcctggatctgagctgtgagaagtccaatcccaagaagccaatcacagagattcaggattcaaaagattcttctcttttggagagtgatatgaaacggaaaggcaagctaaagaataaaggaagcaaaaggaagaaagaagatcttcaggaagtagatggagaaattgaagctgtcctacaaaagaaagctaaagccagggggttagaattccagatctccaacgtgaagtttgaagatgtgggaggcaatgatatgacattaaaagaggtctgcaagatgctcatacacatgcgtcacccggaggtgtaccaccacctgggcgtcgtgccccctcgtggagttctccttcatggaccaccaggctgtgggaagacattacttgcacatgcaattgctggggaacttgacctgccaattttgaaagtggctgctccagagattgtgtctggagtatccggagagtctgagcagaagctgagagaactatttgagcaagctgtgtcaaatgcaccatgtatcattttcattgatgaaattgatgctattacccccaaaagagaagtggcttcaaaagatatggaacgaagaattgtagcccaactcctaacctgcatggatgatctgaataatgtggctgctacagcccgggtcctagttattggagctactaatcgaccagactcgttagaccctgctttgagacgtgcgggaaggttcgaccgagaaatatgcctaggtatcccagatgaagcatccagggaaagaatacttcaaacattgtgcagaaaactgaggcttcctcaagcttttgatttctgtcacttagcacacctaactccaggctttgttggtgctgatctcatggcactgtgccgagaggcagcaatgtgtgcagtcaatagagtcttaatgaagctacaggaacagcagaagaaaaatcctgaaatggaagatttgccatctaaaggagtccaggaggaaaggctgggaactgagcccacttctgaaacacaggatgaattacaaaggctgctggggttgctaagagaccaagatcccctctcagaggagcagatgcaaggactgtgcattgaactgaatgatttcattgttgctctatcctcagtccaaccctctgccaaaagggaaggctttgtcactgtccctaatgtgacatgggcagatattggtgccctggaagacattagagaggagctcaccatggcaatattggcaccagtacgcaacccagaccagttcaaagctcttggattggtgactccagctggggtcctccttgctggtcctcctggctgtgggaagactctgctggcgaaggctgttgcaaatgagtccggactaaattttatatctgtcaagggccccgaattactaaacatgtatgttggtgagagtgaacgtgctgtgcgacaagtttttcaacgagccaagaactcagcaccctgtgtgatattctttgatgaagtggatgctttatgtcctcgaagatcagaccgagagacaggggcaagtgtccgagtggtgaatcagctacttacagagatggatggtctggaagcacgccagcaggtttttattatggcagccactaacaggccagatataattgaccctgcaatcctgcgcccgggccgcctggacaaaacactgtttgtgggtttaccgccccctgcagatcgccttgccatcttaaaaactatcacaaaaaatggtaccaaaccaccactggatgcagatgtaaatttggaagcaattgctggtgaccttcgctgtgattgctatacgggcgcagatctctctgctttggtacgagaagcttctatctgtgccctgagacaggaaatggcaagacagaagagtggaaatgaaaaaggtgaactcaaggttagtcataagcattttgaagaagctttcaagaaagtaagatcatctatatcaaaaaaggatcaaatcatgtatgaacgtttgcaggagtccctcagccgggtcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactata

SEQ ID NO: 10 - protein, 864 aa, Artificial Sequence
  NVL[human]-IF1-H6
MKPRPAGFVDNKLKQRVIQYLTSNKCGKYVDIGVLASDLQRVYSIDYGRRKRNAFRIQVEKVFSIISSEKELKNLTELEDEHLAKRARQGEEDNEYTESYSDDDSSMEDYPDPQSANHMNSSLLSLYRKGNPDSVSNTPEMEQRETTSSTPRISSKTGSIPLKTPAKDSEGGWFIDKTPSVKKDSFFLDLSCEKSNPKKPITEIQDSKDSSLLESDMKRKGKLKNKGSKRKKEDLQEVDGEIEAVLQKKAKARGLEFQISNVKFEDVGGNDMTLKEVCKMLIHMRHPEVYHHLGVVPPRGVLLHGPPGCGKTLLAHAIAGELDLPILKVAAPEIVSGVSGESEQKLRELFEQAVSNAPCIIFIDEIDAITPKREVASKDMERRIVAQLLTCMDDLNNVAATARVLVIGATNRPDSLDPALRRAGRFDREICLGIPDEASRERILQTLCRKLRLPQAFDFCHLAHLTPGFVGADLMALCREAAMCAVNRVLMKLQEQQKKNPEMEDLPSKGVQEERLGTEPTSETQDELQRLLGLLRDQDPLSEEQMQGLCIELNDFIVALSSVQPSAKREGFVTVPNVTWADIGALEDIREELTMAILAPVRNPDQFKALGLVTPAGVLLAGPPGCGKTLLAKAVANESGLNFISVKGPELLNMYVGESERAVRQVFQRAKNSAPCVIFFDEVDALCPRRSDRETGASVRVVNQLLTEMDGLEARQQVFIMAATNRPDIIDPAILRPGRLDKTLFVGLPPPADRLAILKTITKNGTKPPLDADVNLEAIAGDLRCDCYTGADLSALVREASICALRQEMARQKSGNEKGELKVSHKHFEEAFKKVRSSISKKDQIMYERLQESLSRVEHHHHHH

SEQ ID NO: 11 - protein, 341 aa, Homo sapiens
MPKRKVTFQGVGDEEDEDEIIVPKKKLVDPVAGSGGPGSRFKGKHSLDSDEEEDDDDGGSSKYDILASEDVEGQEAATLPSEGGVRITPFNLQEEMEEGHFDADGNYFLNRDAQIRDSWLDNIDWVKIRERPPGQRQASDSEEEDSLGQTSMSAQALLEGLLELLLPRETVAGALRRLGARGGGKGRKGPGQPSSPQRLDRLSGLADQMVARGNLGVYQETRERLAMRLKGLGCQTLGPHNPTPPPSLDMFAEELAEEELETPTPTQRGEAESRGDGLVDVMWEYKWENTGDAELYGPFTSAQMQTWVSEGYFPDGVYCRKLDPPGGQFYNSKRIDFDLYT

SEQ ID NO: 12 - DNA, 1026 bp, Artificial Sequence
  CD2BP2, Acc. BC001947
atgccaaagaggaaagtgaccttccaaggcgtgggagatgaggaggatgaggatgaaatcattgtccccaagaagaagctggtggaccctgtggctgggtcagggggtcctgggagccgctttaaaggcaaacactctttggatagcgatgaggaggaggatgatgatgatggggggtccagcaaatatgacatcttggcctcagaggatgtagaaggtcaggaggcagccacactccccagcgaggggggtgttcggatcacaccctttaacctgcaggaggagatggaggaaggccactttgatgccgatggcaactacttcctgaaccgggatgctcagatccgagacagctggctggacaacattgactgggtgaagatccgggagcggccacctggccagcgccaggcctcagactcggaggaggaggacagcttgggccagacctcaatgagtgcccaagccctcttggagggacttttggagctcctattgcctagagagacagtggctggggcactgaggcgtctgggggcccgaggaggaggcaaagggagaaaggggcctgggcaacccagttcccctcagcgcctggaccggctctccgggttggccgaccagatggtggcccggggcaaccttggtgtgtaccaggaaacaagggaacggttggctatgcgtctgaagggtttggggtgtcagaccctaggaccccacaatcccacacccccaccctccctggacatgttcgctgaggagttggcggaggaggaactggagaccccaacccctacccagagaggagaagcagagtcgcggggagatggtctggtggatgtgatgtgggaatataagtgggagaacacgggggatgccgagctgtatgggcccttcaccagcgcccagatgcagacctgggtgagtgaaggctacttcccggacggtgtttattgccggaagctggacccccctggtggtcagttctacaactccaaacgcattgactttgacctctacacctga

SEQ ID NO: 13 - DNA, 543 bp, Artificial Sequence
  CD2BP2, gene synthesis fragment 1, aa1-173
agcgaagacggcatgccaaagaggaaagtgaccttccaaggcgtgggagatgaggaggatgaggatgaaatcattgtccccaagaagaagctggtggaccctgtggctgggtcagggggtcctgggagccgctttaaaggcaaacactctttggatagcgatgaggaggaggatgatgatgatggggggtccagcaaatatgacatcttggcctcagaggatgtagaaggtcaggaggcagccacactccccagcgaggggggtgttcggatcacaccctttaacctgcaggaggagatggaggaaggccactttgatgccgatggcaactacttcctgaaccgggatgctcagatccgagacagctggctggacaacattgactgggtgaagatccgggagcggccacctggccagcgccaggcctcagactcggaggaggaggacagcttgggccagacctcaatgagtgcccaagccctcttggagggacttttggagctcctattgcctagagagacagtggctggggccgtcttcacc

SEQ ID NO: 14 - DNA, 536 bp, Artificial Sequence
  CD2BP2, gene synthesis fragment 2, aa174-341
agcgaagacccggggcactgaggcgtctgggggcccgaggaggaggcaaagggagaaaggggcctgggcaacccagttcccctcagcgcctggaccggctctccgggttggccgaccagatggtggcccggggcaaccttggtgtgtaccaggaaacaagggaacggttggctatgcgtctgaagggtttggggtgtcagaccctaggaccccacaatcccacacccccaccctccctggacatgttcgctgaggagttggcggaggaggaactggagaccccaacccctacccagagaggagaagcagagtcgcggggagatggtctggtggatgtgatgtgggaatataagtgggagaacacgggggatgccgagctgtatgggcccttcaccagcgcccagatgcagacctgggtgagtgaaggctacttcccggacggtgtttattgccggaagctggacccccctggtggtcagttctacaactccaaacgcattgactttgacctctacacctgatcgaccgtcttctca

SEQ ID NO: 15 - DNA, 517 bp, Artificial Sequence
  CD2BP2, gene synthesis fragment 1, BbsI
catgccaaagaggaaagtgaccttccaaggcgtgggagatgaggaggatgaggatgaaatcattgtccccaagaagaagctggtggaccctgtggctgggtcagggggtcctgggagccgctttaaaggcaaacactctttggatagcgatgaggaggaggatgatgatgatggggggtccagcaaatatgacatcttggcctcagaggatgtagaaggtcaggaggcagccacactccccagcgaggggggtgttcggatcacaccctttaacctgcaggaggagatggaggaaggccactttgatgccgatggcaactacttcctgaaccgggatgctcagatccgagacagctggctggacaacattgactgggtgaagatccgggagcggccacctggccagcgccaggcctcagactcggaggaggaggacagcttgggccagacctcaatgagtgcccaagccctcttggagggacttttggagctcctattgcctagagagacagtggct

SEQ ID NO: 16 - DNA, 510 bp, Artificial Sequence
  CD2BP2, gene synthesis fragment 2, BbsI
ggggcactgaggcgtctgggggcccgaggaggaggcaaagggagaaaggggcctgggcaacccagttcccctcagcgcctggaccggctctccgggttggccgaccagatggtggcccggggcaaccttggtgtgtaccaggaaacaagggaacggttggctatgcgtctgaagggtttggggtgtcagaccctaggaccccacaatcccacacccccaccctccctggacatgttcgctgaggagttggcggaggaggaactggagaccccaacccctacccagagaggagaagcagagtcgcggggagatggtctggtggatgtgatgtgggaatataagtgggagaacacgggggatgccgagctgtatgggcccttcaccagcgcccagatgcagacctgggtgagtgaaggctacttcccggacggtgtttattgccggaagctggacccccctggtggtcagttctacaactccaaacgcattgactttgacctctacacctga

SEQ ID NO: 17 - DNA, 39 bp, Artificial Sequence
  sense CD2BP2-fragment-2
atagaagacccggggcactgaggcgtctgggggcccgag

SEQ ID NO: 18 - DNA, 39 bp, Artificial Sequence
  asense CD2BP2-fragment-2
tatgaagacggtcgagggtgtagaggtcaaagtcaatgc

SEQ ID NO: 19 - DNA, 534 bp, Artificial Sequence
  CD2BP2-PCR-fragment
atagaagacccggggcactgaggcgtctgggggcccgaggaggaggcaaagggagaaaggggcctgggcaacccagttcccctcagcgcctggaccggctctccgggttggccgaccagatggtggcccggggcaaccttggtgtgtaccaggaaacaagggaacggttggctatgcgtctgaagggtttggggtgtcagaccctaggaccccacaatcccacacccccaccctccctggacatgttcgctgaggagttggcggaggaggaactggagaccccaacccctacccagagaggagaagcagagtcgcggggagatggtctggtggatgtgatgtgggaatataagtgggagaacacgggggatgccgagctgtatgggcccttcaccagcgcccagatgcagacctgggtgagtgaaggctacttcccggacggtgtttattgccggaagctggacccccctggtggtcagttctacaactccaaacgcattgactttgacctctacaccctcgaccgtcttcata

SEQ ID NO: 20 - DNA, 6637 bp, Artificial Sequence
  pTriEx-1-CD2BP2[human]
ggagtcgctgcgacgctgccttcgccccgtgccccgctccgccgccgcctcgcgccgcccgccccggctctgactgaccgcgttactcccacaggtgagcgggcgggacggcccttctccttcgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcggaccgaaattaatacgactcactataggggaattgtgagcggataacaattccccggagttaatccgggacctttaattcaacccaacacaatatattatagttaaataagaattattatcaaatcatttgtatattaattaaaatactatactgtaaattacattttatttacaatcaaaggagatataccatgccaaagaggaaagtgaccttccaaggcgtgggagatgaggaggatgaggatgaaatcattgtccccaagaagaagctggtggaccctgtggctgggtcagggggtcctgggagccgctttaaaggcaaacactctttggatagcgatgaggaggaggatgatgatgatggggggtccagcaaatatgacatcttggcctcagaggatgtagaaggtcaggaggcagccacactccccagcgaggggggtgttcggatcacaccctttaacctgcaggaggagatggaggaaggccactttgatgccgatggcaactacttcctgaaccgggatgctcagatccgagacagctggctggacaacattgactgggtgaagatccgggagcggccacctggccagcgccaggcctcagactcggaggaggaggacagcttgggccagacctcaatgagtgcccaagccctcttggagggacttttggagctcctattgcctagagagacagtggctggggcactgaggcgtctgggggcccgaggaggaggcaaagggagaaaggggcctgggcaacccagttcccctcagcgcctggaccggctctccgggttggccgaccagatggtggcccggggcaaccttggtgtgtaccaggaaacaagggaacggttggctatgcgtctgaagggtttggggtgtcagaccctaggaccccacaatcccacacccccaccctccctggacatgttcgctgaggagttggcggaggaggaactggagaccccaacccctacccagagaggagaagcagagtcgcggggagatggtctggtggatgtgatgtgggaatataagtgggagaacacgggggatgccgagctgtatgggcccttcaccagcgcccagatgcagacctgggtgagtgaaggctacttcccggacggtgtttattgccggaagctggacccccctggtggtcagttctacaactccaaacgcattgactttgacctctacaccctcgagcaccaccatcaccatcaccatcactaagtgattaacctcaggtgcaggctgcctatcagaaggtggtggctggtgtggccaatgccctggctcacaaataccactgagatcgatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgtaactagcataaccccttggggcctctaaacgggtcttgaggggttttttgctgaaagcatgcggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaatagagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggaacgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgtccgcgcgtttcctgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgtcaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcttcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgcatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 21 - protein, 351 aa, Artificial Sequence
  CD2BP2[human]-H8
MPKRKVTFQGVGDEEDEDEIIVPKKKLVDPVAGSGGPGSRFKGKHSLDSDEEEDDDDGGSSKYDILASEDVEGQEAATLPSEGGVRITPFNLQEEMEEGHFDADGNYFLNRDAQIRDSWLDNIDWVKIRERPPGQRQASDSEEEDSLGQTSMSAQALLEGLLELLLPRETVAGALRRLGARGGGKGRKGPGQPSSPQRLDRLSGLADQMVARGNLGVYQETRERLAMRLKGLGCQTLGPHNPTPPPSLDMFAEELAEEELETPTPTQRGEAESRGDGLVDVMWEYKWENTGDAELYGPFTSAQMQTWVSEGYFPDGVYCRKLDPPGGQFYNSKRIDFDLYTLEHHHHHHHH

SEQ ID NO: 22 - DNA, 6273 bp, Artificial Sequence
  pET24d-N-CD2BP2[human]
ggggaattgtgagcggataacaattcccctctagaaataattttgtttaactttaagaaggagatataccatgagccatcatcatcatcatcatcatcattccatgccaaagaggaaagtgaccttccaaggcgtgggagatgaggaggatgaggatgaaatcattgtccccaagaagaagctggtggaccctgtggctgggtcagggggtcctgggagccgctttaaaggcaaacactctttggatagcgatgaggaggaggatgatgatgatggggggtccagcaaatatgacatcttggcctcagaggatgtagaaggtcaggaggcagccacactccccagcgaggggggtgttcggatcacaccctttaacctgcaggaggagatggaggaaggccactttgatgccgatggcaactacttcctgaaccgggatgctcagatccgagacagctggctggacaacattgactgggtgaagatccgggagcggccacctggccagcgccaggcctcagactcggaggaggaggacagcttgggccagacctcaatgagtgcccaagccctcttggagggacttttggagctcctattgcctagagagacagtggctggggcactgaggcgtctgggggcccgaggaggaggcaaagggagaaaggggcctgggcaacccagttcccctcagcgcctggaccggctctccgggttggccgaccagatggtggcccggggcaaccttggtgtgtaccaggaaacaagggaacggttggctatgcgtctgaagggtttggggtgtcagaccctaggaccccacaatcccacacccccaccctccctggacatgttcgctgaggagttggcggaggaggaactggagaccccaacccctacccagagaggagaagcagagtcgcggggagatggtctggtggatgtgatgtgggaatataagtgggagaacacgggggatgccgagctgtatgggcccttcaccagcgcccagatgcagacctgggtgagtgaaggctacttcccggacggtgtttattgccggaagctggacccccctggtggtcagttctacaactccaaacgcattgactttgacctctacacctgatcgagtgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactata

SEQ ID NO: 23 - protein, 352 aa, Artificial Sequence
  H8-CD2BP2[human]
MSHHHHHHHHSMPKRKVTFQGVGDEEDEDEIIVPKKKLVDPVAGSGGPGSRFKGKHSLDSDEEEDDDDGGSSKYDILASEDVEGQEAATLPSEGGVRITPFNLQEEMEEGHFDADGNYFLNRDAQIRDSWLDNIDWVKIRERPPGQRQASDSEEEDSLGQTSMSAQALLEGLLELLLPRETVAGALRRLGARGGGKGRKGPGQPSSPQRLDRLSGLADQMVARGNLGVYQETRERLAMRLKGLGCQTLGPHNPTPPPSLDMFAEELAEEELETPTPTQRGEAESRGDGLVDVMWEYKWENTGDAELYGPFTSAQMQTWVSEGYFPDGVYCRKLDPPGGQFYNSKRIDFDLYT

SEQ ID NO: 24 - protein, 1668 aa, Homo sapiens
MSARAAAAKSTAMEETAIWEQHTVTLHRAPGFGFGIAISGGRDNPHFQSGETSIVISDVLKGGPAEGQLQENDRVAMVNGVSMDNVEHAFAVQQLRKSGKNAKITIRRKKKVQIPVSRPDPEPVSDNEEDSYDEEIHDPRSGRSGVVNRRSEKIWPRDRSASRERSLSPRSDRRSVASSQPAKPTKVTLVKSRKNEEYGLRLASHIFVKEISQDSLAARDGNIQEGDVVLKINGTVTENMSLTDAKTLIERSKGKLKMVVQRDERATLLNVPDLSDSIHSANASERDDISEIQSLASDHSGRSHDRPPRRSRSRSPDQRSEPSDHSRHSPQQPSNGSLRSRDEERISKPGAVSTPVKHADDHTPKTVEEVTVERNEKQTPSLPEPKPVYAQVGQPDVDLPVSPSDGVLPNSTHEDGILRPSMKLVKFRKGDSVGLRLAGGNDVGIFVAGVLEDSPAAKEGLEEGDQILRVNNVDFTNIIREEAVLFLLDLPKGEEVTILAQKKKDVYRRIVESDVGDSFYIRTHFEYEKESPYGLSFNKGEVFRVVDTLYNGKLGSWLAIRIGKNHKEVERGIIPNKNRAEQLASVQYTLPKTAGGDRADFWRFRGLRSSKRNLRKSREDLSAQPVQTKFPAYERVVLREAGFLRPVTIFGPIADVAREKLAREEPDIYQIAKSEPRDAGTDQRSSGIIRLHTIKQIIDQDKHALLDVTPNAVDRLNYAQWYPIVVFLNPDSKQGVKTMRMRLCPESRKSARKLYERSHKLRKNNHHLFTTTINLNSMNDGWYGALKEAIQQQQNQLVWVSEGKADGATSDDLDLHDDRLSYLSAPGSEYSMYSTDSRHTSDYEDTDTEGGAYTDQELDETLNDEVGTPPESAITRSSEPVREDSSGMHHENQTYPPYSPQAQPQPIHRIDSPGFKPASQQVYRKDPYPEEMMRQNHVLKQPAVSHPGHRPDKEPNLTYEPQLPYVEKQASRDLEQPTYRYESSSYTDQFSRNYEHRLRYEDRVPMYEEQWSYYDDKQPYPSRPPFDNQHSQDLDSRQHPEESSERGYFPRFEEPAPLSYDSRPRYEQAPRASALRHEEQPAPGYDTHGRLRPEAQPHPSAGPKPAESKQYFEQYSRSYEQVPPQGFTSRAGHFEPLHGAAAVPPLIPSSQHKPEALPSNTKPLPPPPTQTEEEEDPAMKPQSVLTRVKMFENKRSASLETKKDVNDTGSFKPPEVASKPSGAPIIGPKPTSQNQFSEHDKTLYRIPEPQKPQLKPPEDIVRSNHYDPEEDEEYYRKQLSYFDRRSFENKPPAHIAASHLSEPAKPAHSQNQSNFSSYSSKGKPPEADGVDRSFGEKRYEPIQATPPPPPLPSQYAQPSQPVTSASLHIHSKGAHGEGNSVSLDFQNSLVSKPDPPPSQNKPATFRPPNREDTAQAAFYPQKSFPDKAPVNGTEQTQKTVTPAYNRFTPKPYTSSARPFERKFESPKFNHNLLPSETAHKPDLSSKTPTSPKTLVKSHSLAQPPEFDSGVETFSIHAEKPKYQINNISTVPKAIPVSPSAVEEDEDEDGHTVVATARGIFNSNGGVLSSIETGVSIIIPQGAIPEGVEQEIYFKVCRDNSILPPLDKEKGETLLSPLVMCGPHGLKFLKPVELRLPHCDPKTWQNKCLPGDPNYLVGANCVSVLIDHF

SEQ ID NO: 25 - DNA, 33 bp, Artificial Sequence
  sense TJP1-IF2-ZF1
ataggtctctcatgtccgccagagctgcggctg

SEQ ID NO: 26 - DNA, 38 bp, Artificial Sequence
  asense TJP1-IF2-ZF1
ataggtctctacacctgttgagaggctggcttaaatcc

SEQ ID NO: 27 - DNA, 35 bp, Artificial Sequence
  sense TJP1-IF2-ZF2
atacgtctcagtgtatagaaaggatccatatcccg

SEQ ID NO: 28 - DNA, 41 bp, Artificial Sequence
  asense TJP1-IF2-ZF2
atacgtctcctcgagaaagtggtcaataaggacagaaacac

SEQ ID NO: 29 - DNA, 2788 bp, Artificial Sequence
  TJP1-PCR-fragment-1
ataggtctctcatgtccgccagagctgcggctgccaagagcacagcaatggaggaaacagctatatgggaacaacatacagtgacgcttcacagggctcctggatttggatttggaattgcaatatctggtggacgagataatcctcattttcagagtggggaaacgtcaatagtgatttcagatgtgctgaaaggaggaccagctgaaggacagctacaggaaaatgaccgagttgcaatggttaacggagtttcaatggataatgttgaacatgcttttgctgttcagcaactaaggaaaagtgggaaaaatgcaaaaattacaattagaaggaagaagaaagttcaaataccagtaagtcgtcctgatcctgaaccagtatctgataatgaagaagatagttatgatgaggaaatacatgatccaagaagtggccggagtggtgtggttaacagaaggagtgagaagatttggccgagggatagaagtgcaagtagagagaggagcttgtccccgcggtcagacaggcggtcagtggcttccagccagcctgctaaacctactaaagtcacactggtgaaatcccggaaaaatgaagaatatggtcttcgattggcaagccatatatttgttaaggaaatttcacaagatagtttggcagcaagagatggcaatattcaagaaggtgatgttgtattgaagataaatggtactgtgacagaaaatatgtcattgacagatgcaaagacattgatagaaaggtctaaaggcaaattaaaaatggtagttcaaagagatgaacgggctacgctattgaatgtccctgatctttctgacagcatccactctgctaatgcctctgagagagacgacatttcagaaattcagtcactggcatcagatcattctggtcgatcacacgataggcctccccgccgcagccggtcacgatctcctgaccagcggtcagagccttctgatcattccaggcactcgccgcagcagccaagcaatggcagtctccggagtagagatgaagagagaatttctaaacctggggctgtctcaactcctgtaaagcatgctgatgatcacacacctaaaacagtggaagaagttacagttgaaagaaatgagaaacaaacaccttctcttccagaaccaaagcctgtgtatgcccaagttgggcaaccagatgtggatttacctgtcagtccatctgatggtgtcctacctaattcaactcatgaagatgggattcttcggcccagcatgaaattggtaaaattcagaaaaggagatagtgtgggtttgcggctggctggtggaaatgatgttggaatatttgtagctggcgttctagaagatagccctgcagccaaggaaggcttagaggaaggtgatcaaattctcagggtaaacaacgtagattttacaaatatcataagagaagaagccgtccttttcctgcttgacctccctaaaggagaagaagtgaccatattggctcagaagaagaaggatgtttatcgtcgcattgtagaatcagatgtaggagattctttctatattagaacccattttgaatatgaaaaggaatctccctatggacttagttttaacaaaggagaggtgttccgtgttgtggataccttgtacaatggaaaactgggctcttggcttgctattcgaattggtaaaaatcataaggaggtagaacgaggcatcatccctaataagaacagagctgagcagctagccagtgtacagtatacacttccaaaaacagcaggcggagaccgtgctgacttctggagattcagaggtcttcgcagctccaagagaaatcttcgaaaaagcagagaggatttgtccgctcagcctgttcaaacaaagtttccagcttatgaaagagtggttcttcgagaagctggatttctgaggcctgtaaccatttttggaccaatagctgatgttgccagagaaaagctggcaagagaagaaccagatatttatcaaattgcaaagagtgaaccacgagacgctggaactgaccaacgtagctctggcattattcgcctgcatacaataaagcaaatcatagatcaagacaaacatgctttattagatgtaacaccaaatgcagttgatcgtcttaactatgcccagtggtatccaattgttgtatttcttaaccctgattctaagcaaggagtaaaaacaatgagaatgaggttatgtccagaatctcggaaaagtgccaggaagttatacgagcgatctcataaacttcgtaaaaataatcaccatctttttacaactacaattaacttaaattcaatgaatgatggttggtatggtgcgctgaaagaagcaattcaacaacagcaaaaccagctggtatgggtttccgagggaaaggcggatggtgctacaagtgatgaccttgatttgcatgatgatcgtctgtcctacctgtcagctccaggtagtgaatactcaatgtatagcacggacagtagacacacttctgactatgaagacacagacacagaaggcggggcctacactgatcaagaactagatgaaactcttaatgatgaggttgggactccaccggagtctgccattacacggtcctctgagcctgtaagagaggactcctctggaatgcatcatgaaaaccaaacatatcctccttactcaccacaagcgcagccacaaccaattcatagaatagactcccctggatttaagccagcctctcaacaggtgtagagacctat

SEQ ID NO: 30 - DNA, 2266 bp, Artificial Sequence
  TJP1-PCR-fragment-2
atacgtctcagtgtatagaaaggatccatatcccgaggaaatgatgaggcagaaccatgttttgaaacagccagccgttagtcacccagggcacaggccagacaaagagcctaatctgacctatgaaccccaactcccatacgtagagaaacaagccagcagagacctcgagcagcccacatacagatacgagtcctcaagctatacggaccagttttctcgaaactatgaacatcgtctgcgatacgaagatcgcgtccccatgtatgaagaacagtggtcatattatgatgacaaacagccctacccatctcggccaccttttgataatcagcactctcaagaccttgactccagacagcatcccgaagagtcctcagaacgagggtactttccacgttttgaagagccagcccctctgtcttacgacagcagaccacgttacgaacaggcacctagagcatccgccctgcggcacgaagagcagccagctcctgggtatgacacacatggtagactcagaccggaagcccagccccacccttcagcagggcccaagcctgcagagtccaagcagtattttgagcaatattcacgcagttacgagcaagtaccaccccaaggatttacctctagagcaggtcattttgagcctctccatggtgctgcagctgtccctccgctgataccttcatctcagcataagccagaagctctgccttcaaacaccaaaccactgcctccacccccaactcaaaccgaagaagaggaagatccagcaatgaagccacagtctgtactcaccagagttaagatgtttgaaaacaaaagatctgcatccttagagaccaagaaggatgtaaatgacactggcagttttaagcctccagaagtagcatctaaaccttcaggtgctcccatcattggtcccaaacccacttctcagaatcaattcagtgaacatgacaaaactctgtacaggatcccagaacctcaaaaacctcaactgaagccacctgaagatattgttcggtccaatcattatgaccctgaagaagatgaagaatattatcgaaaacagctgtcatactttgaccgaagaagttttgagaataagcctcctgcacacattgctgccagccatctctccgagcctgcaaagccagctcattctcagaatcaatcaaatttttctagttattcttcaaagggaaagcctcctgaagctgatggtgtggatagatcatttggcgagaaacgctatgaacccatccaggccactccccctcctcctccattgccctcgcagtatgcccagccatctcagcctgtcaccagcgcgtctctccacatacattctaagggagcacatggtgaaggtaattcagtgtcattggattttcagaattccttagtgtccaaaccagacccacctccatctcagaataagccagcaactttcagaccaccaaaccgagaagatactgctcaggcagctttctatccccagaaaagttttccagataaagccccagttaatggaactgaacagactcagaaaacagtcactccagcatacaatcgattcacaccaaaaccatatacaagttctgcccgaccatttgaacgcaagtttgaaagtcctaaattcaatcacaatcttctgccaagtgaaactgcacataaacctgacttgtcttcaaaaactcccacttctccaaaaactcttgtgaaatcgcacagtttggcacagcctcctgagtttgacagtggagttgaaactttctctatccatgcagagaagcctaaatatcaaataaataatatcagcacagtgcctaaagctattcctgtgagtccttcagctgtggaagaggatgaagatgaagatggtcatactgtggtggccacagcccgaggcatatttaacagcaatgggggcgtgctgagttccatagaaactggtgttagtataattatccctcaaggagccattcccgaaggagttgagcaggaaatctatttcaaggtctgccgggacaacagcatccttccacctttagataaagagaaaggtgaaacactgctgagtcctttggtgatgtgtggtccccatggcctcaagttcctgaagcctgtggagctgcgcttaccacactgtgatcctaaaacctggcaaaacaagtgtcttcccggagatccaaattatctcgttggagcaaactgtgtttctgtccttattgaccactttctcgaggagacgtat

SEQ ID NO: 31 - DNA, 10618 bp, Artificial Sequence
  pTriEx-1-TJP1[human]-IF2
ggggaattgtgagcggataacaattccccggagttaatccgggacctttaattcaacccaacacaatatattatagttaaataagaattattatcaaatcatttgtatattaattaaaatactatactgtaaattacattttatttacaatcaaaggagatataccatgtccgccagagctgcggctgccaagagcacagcaatggaggaaacagctatatgggaacaacatacagtgacgcttcacagggctcctggatttggatttggaattgcaatatctggtggacgagataatcctcattttcagagtggggaaacgtcaatagtgatttcagatgtgctgaaaggaggaccagctgaaggacagctacaggaaaatgaccgagttgcaatggttaacggagtttcaatggataatgttgaacatgcttttgctgttcagcaactaaggaaaagtgggaaaaatgcaaaaattacaattagaaggaagaagaaagttcaaataccagtaagtcgtcctgatcctgaaccagtatctgataatgaagaagatagttatgatgaggaaatacatgatccaagaagtggccggagtggtgtggttaacagaaggagtgagaagatttggccgagggatagaagtgcaagtagagagaggagcttgtccccgcggtcagacaggcggtcagtggcttccagccagcctgctaaacctactaaagtcacactggtgaaatcccggaaaaatgaagaatatggtcttcgattggcaagccatatatttgttaaggaaatttcacaagatagtttggcagcaagagatggcaatattcaagaaggtgatgttgtattgaagataaatggtactgtgacagaaaatatgtcattgacagatgcaaagacattgatagaaaggtctaaaggcaaattaaaaatggtagttcaaagagatgaacgggctacgctattgaatgtccctgatctttctgacagcatccactctgctaatgcctctgagagagacgacatttcagaaattcagtcactggcatcagatcattctggtcgatcacacgataggcctccccgccgcagccggtcacgatctcctgaccagcggtcagagccttctgatcattccaggcactcgccgcagcagccaagcaatggcagtctccggagtagagatgaagagagaatttctaaacctggggctgtctcaactcctgtaaagcatgctgatgatcacacacctaaaacagtggaagaagttacagttgaaagaaatgagaaacaaacaccttctcttccagaaccaaagcctgtgtatgcccaagttgggcaaccagatgtggatttacctgtcagtccatctgatggtgtcctacctaattcaactcatgaagatgggattcttcggcccagcatgaaattggtaaaattcagaaaaggagatagtgtgggtttgcggctggctggtggaaatgatgttggaatatttgtagctggcgttctagaagatagccctgcagccaaggaaggcttagaggaaggtgatcaaattctcagggtaaacaacgtagattttacaaatatcataagagaagaagccgtccttttcctgcttgacctccctaaaggagaagaagtgaccatattggctcagaagaagaaggatgtttatcgtcgcattgtagaatcagatgtaggagattctttctatattagaacccattttgaatatgaaaaggaatctccctatggacttagttttaacaaaggagaggtgttccgtgttgtggataccttgtacaatggaaaactgggctcttggcttgctattcgaattggtaaaaatcataaggaggtagaacgaggcatcatccctaataagaacagagctgagcagctagccagtgtacagtatacacttccaaaaacagcaggcggagaccgtgctgacttctggagattcagaggtcttcgcagctccaagagaaatcttcgaaaaagcagagaggatttgtccgctcagcctgttcaaacaaagtttccagcttatgaaagagtggttcttcgagaagctggatttctgaggcctgtaaccatttttggaccaatagctgatgttgccagagaaaagctggcaagagaagaaccagatatttatcaaattgcaaagagtgaaccacgagacgctggaactgaccaacgtagctctggcattattcgcctgcatacaataaagcaaatcatagatcaagacaaacatgctttattagatgtaacaccaaatgcagttgatcgtcttaactatgcccagtggtatccaattgttgtatttcttaaccctgattctaagcaaggagtaaaaacaatgagaatgaggttatgtccagaatctcggaaaagtgccaggaagttatacgagcgatctcataaacttcgtaaaaataatcaccatctttttacaactacaattaacttaaattcaatgaatgatggttggtatggtgcgctgaaagaagcaattcaacaacagcaaaaccagctggtatgggtttccgagggaaaggcggatggtgctacaagtgatgaccttgatttgcatgatgatcgtctgtcctacctgtcagctccaggtagtgaatactcaatgtatagcacggacagtagacacacttctgactatgaagacacagacacagaaggcggggcctacactgatcaagaactagatgaaactcttaatgatgaggttgggactccaccggagtctgccattacacggtcctctgagcctgtaagagaggactcctctggaatgcatcatgaaaaccaaacatatcctccttactcaccacaagcgcagccacaaccaattcatagaatagactcccctggatttaagccagcctctcaacaggtgtatagaaaggatccatatcccgaggaaatgatgaggcagaaccatgttttgaaacagccagccgttagtcacccagggcacaggccagacaaagagcctaatctgacctatgaaccccaactcccatacgtagagaaacaagccagcagagacctcgagcagcccacatacagatacgagtcctcaagctatacggaccagttttctcgaaactatgaacatcgtctgcgatacgaagatcgcgtccccatgtatgaagaacagtggtcatattatgatgacaaacagccctacccatctcggccaccttttgataatcagcactctcaagaccttgactccagacagcatcccgaagagtcctcagaacgagggtactttccacgttttgaagagccagcccctctgtcttacgacagcagaccacgttacgaacaggcacctagagcatccgccctgcggcacgaagagcagccagctcctgggtatgacacacatggtagactcagaccggaagcccagccccacccttcagcagggcccaagcctgcagagtccaagcagtattttgagcaatattcacgcagttacgagcaagtaccaccccaaggatttacctctagagcaggtcattttgagcctctccatggtgctgcagctgtccctccgctgataccttcatctcagcataagccagaagctctgccttcaaacaccaaaccactgcctccacccccaactcaaaccgaagaagaggaagatccagcaatgaagccacagtctgtactcaccagagttaagatgtttgaaaacaaaagatctgcatccttagagaccaagaaggatgtaaatgacactggcagttttaagcctccagaagtagcatctaaaccttcaggtgctcccatcattggtcccaaacccacttctcagaatcaattcagtgaacatgacaaaactctgtacaggatcccagaacctcaaaaacctcaactgaagccacctgaagatattgttcggtccaatcattatgaccctgaagaagatgaagaatattatcgaaaacagctgtcatactttgaccgaagaagttttgagaataagcctcctgcacacattgctgccagccatctctccgagcctgcaaagccagctcattctcagaatcaatcaaatttttctagttattcttcaaagggaaagcctcctgaagctgatggtgtggatagatcatttggcgagaaacgctatgaacccatccaggccactccccctcctcctccattgccctcgcagtatgcccagccatctcagcctgtcaccagcgcgtctctccacatacattctaagggagcacatggtgaaggtaattcagtgtcattggattttcagaattccttagtgtccaaaccagacccacctccatctcagaataagccagcaactttcagaccaccaaaccgagaagatactgctcaggcagctttctatccccagaaaagttttccagataaagccccagttaatggaactgaacagactcagaaaacagtcactccagcatacaatcgattcacaccaaaaccatatacaagttctgcccgaccatttgaacgcaagtttgaaagtcctaaattcaatcacaatcttctgccaagtgaaactgcacataaacctgacttgtcttcaaaaactcccacttctccaaaaactcttgtgaaatcgcacagtttggcacagcctcctgagtttgacagtggagttgaaactttctctatccatgcagagaagcctaaatatcaaataaataatatcagcacagtgcctaaagctattcctgtgagtccttcagctgtggaagaggatgaagatgaagatggtcatactgtggtggccacagcccgaggcatatttaacagcaatgggggcgtgctgagttccatagaaactggtgttagtataattatccctcaaggagccattcccgaaggagttgagcaggaaatctatttcaaggtctgccgggacaacagcatccttccacctttagataaagagaaaggtgaaacactgctgagtcctttggtgatgtgtggtccccatggcctcaagttcctgaagcctgtggagctgcgcttaccacactgtgatcctaaaacctggcaaaacaagtgtcttcccggagatccaaattatctcgttggagcaaactgtgtttctgtccttattgaccactttctcgagcaccaccatcaccatcaccatcactaagtgattaacctcaggtgcaggctgcctatcagaaggtggtggctggtgtggccaatgccctggctcacaaataccactgagatcgatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgtaactagcataaccccttggggcctctaaacgggtcttgaggggttttttgctgaaagcatgcggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaatagagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggaacgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgtccgcgcgtttcctgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgtcaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcttcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgcatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctccttcgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatcggaccgaaattaatacgactcactata

SEQ ID NO: 32 - protein, 1678 aa, Artificial Sequence
  TJP1[human]-IF2-H8
MSARAAAAKSTAMEETAIWEQHTVTLHRAPGFGFGIAISGGRDNPHFQSGETSIVISDVLKGGPAEGQLQENDRVAMVNGVSMDNVEHAFAVQQLRKSGKNAKITIRRKKKVQIPVSRPDPEPVSDNEEDSYDEEIHDPRSGRSGVVNRRSEKIWPRDRSASRERSLSPRSDRRSVASSQPAKPTKVTLVKSRKNEEYGLRLASHIFVKEISQDSLAARDGNIQEGDVVLKINGTVTENMSLTDAKTLIERSKGKLKMVVQRDERATLLNVPDLSDSIHSANASERDDISEIQSLASDHSGRSHDRPPRRSRSRSPDQRSEPSDHSRHSPQQPSNGSLRSRDEERISKPGAVSTPVKHADDHTPKTVEEVTVERNEKQTPSLPEPKPVYAQVGQPDVDLPVSPSDGVLPNSTHEDGILRPSMKLVKFRKGDSVGLRLAGGNDVGIFVAGVLEDSPAAKEGLEEGDQILRVNNVDFTNIIREEAVLFLLDLPKGEEVTILAQKKKDVYRRIVESDVGDSFYIRTHFEYEKESPYGLSFNKGEVFRVVDTLYNGKLGSWLAIRIGKNHKEVERGIIPNKNRAEQLASVQYTLPKTAGGDRADFWRFRGLRSSKRNLRKSREDLSAQPVQTKFPAYERVVLREAGFLRPVTIFGPIADVAREKLAREEPDIYQIAKSEPRDAGTDQRSSGIIRLHTIKQIIDQDKHALLDVTPNAVDRLNYAQWYPIVVFLNPDSKQGVKTMRMRLCPESRKSARKLYERSHKLRKNNHHLFTTTINLNSMNDGWYGALKEAIQQQQNQLVWVSEGKADGATSDDLDLHDDRLSYLSAPGSEYSMYSTDSRHTSDYEDTDTEGGAYTDQELDETLNDEVGTPPESAITRSSEPVREDSSGMHHENQTYPPYSPQAQPQPIHRIDSPGFKPASQQVYRKDPYPEEMMRQNHVLKQPAVSHPGHRPDKEPNLTYEPQLPYVEKQASRDLEQPTYRYESSSYTDQFSRNYEHRLRYEDRVPMYEEQWSYYDDKQPYPSRPPFDNQHSQDLDSRQHPEESSERGYFPRFEEPAPLSYDSRPRYEQAPRASALRHEEQPAPGYDTHGRLRPEAQPHPSAGPKPAESKQYFEQYSRSYEQVPPQGFTSRAGHFEPLHGAAAVPPLIPSSQHKPEALPSNTKPLPPPPTQTEEEEDPAMKPQSVLTRVKMFENKRSASLETKKDVNDTGSFKPPEVASKPSGAPIIGPKPTSQNQFSEHDKTLYRIPEPQKPQLKPPEDIVRSNHYDPEEDEEYYRKQLSYFDRRSFENKPPAHIAASHLSEPAKPAHSQNQSNFSSYSSKGKPPEADGVDRSFGEKRYEPIQATPPPPPLPSQYAQPSQPVTSASLHIHSKGAHGEGNSVSLDFQNSLVSKPDPPPSQNKPATFRPPNREDTAQAAFYPQKSFPDKAPVNGTEQTQKTVTPAYNRFTPKPYTSSARPFERKFESPKFNHNLLPSETAHKPDLSSKTPTSPKTLVKSHSLAQPPEFDSGVETFSIHAEKPKYQINNISTVPKAIPVSPSAVEEDEDEDGHTVVATARGIFNSNGGVLSSIETGVSIIIPQGAIPEGVEQEIYFKVCRDNSILPPLDKEKGETLLSPLVMCGPHGLKFLKPVELRLPHCDPKTWQNKCLPGDPNYLVGANCVSVLIDHFLEHHHHHHHH

SEQ ID NO: 33 - DNA, 31 bp, Artificial Sequence
  sense NVL-F1
ataggtctcacatgaagccacgtcctgcagg

SEQ ID NO: 34 - DNA, 42 bp, Artificial Sequence
  asense NVL-F1
tatggtctcatcgagtcattagttagctgactggggatcagg

SEQ ID NO: 35 - DNA, 383 bp, Artificial Sequence
  NVL-PCR-fragment 1-117
ataggtctcacatgaagccacgtcctgcaggatttgtggacaacaaactcaagcaacgcgttattcagtatctgaccagtaacaagtgcggcaaatacgtagacataggggttttggcgtctgatctgcagagggtgtactctatcgactatggtcggaggaaacggaatgcctttcgaattcaggtcgaaaaggtgttctccatcatcagctcagagaaagagctgaagaatctcactgaacttgaggacgaacatctcgccaaaagagccagacaaggcgaagaggacaatgagtacacagagtcctacagtgatgatgactcatcaatggaggactatcctgatccccagtcagctaactaatgactcgatgagaccata

SEQ ID NO: 36 - DNA, 37 bp, Artificial Sequence
  sense NVL-F2
ataggtctcacatgtcaatggaggactatcctgatcc

SEQ ID NO: 37 - DNA, 42 bp, Artificial Sequence
  asense NVL-F2
tatggtctcatcgagtcattagtggtggtacacctccgggtg

SEQ ID NO: 38 - DNA, 596 bp, Artificial Sequence
  NVL-PCR-fragment 106-292
ataggtctcacatgtcaatggaggactatcctgatccccagtcagctaaccacatgaactcttctctgcttagcctgtataggaaggggaatcccgatagtgtgagcaacacaccggaaatggagcagagagaaaccaccagttccactccacgcatatcctccaaaaccggtagcattcccttgaaaactcctgctaaagactctgaaggaggctggtttatcgacaaaacgcctagcgtcaagaaggatagcttcttcctggatctgagctgtgagaagtccaatcccaagaagccaatcacagagattcaggattcaaaagattcttctcttttggagagtgatatgaaacggaaaggcaagctaaagaataaaggaagcaaaaggaagaaagaagatcttcaggaagtagatggagaaattgaagctgtcctacaaaagaaagctaaagccagggggttagaattccagatctccaacgtgaagtttgaagatgtgggaggcaatgatatgacattaaaagaggtctgcaagatgctcatacacatgcgtcacccggaggtgtaccaccactaatgactcgatgagaccata

SEQ ID NO: 39 - DNA, 33 bp, Artificial Sequence
  sense NVL-F3
ataggtctcacatgctcatacacatgcgtcacc

SEQ ID NO: 40 - DNA, 43 bp, Artificial Sequence
  asense NVL-F3
tatggtctcatcgagtcattatagagcaacaatgaaatcattc

SEQ ID NO: 41 - DNA, 875 bp, Artificial Sequence
  NVL-PCR-fragment 281-560
ataggtctcacatgctcatacacatgcgtcacccggaggtgtaccaccacctgggcgtcgtgccccctcgtggagttctccttcatggaccaccaggctgtgggaagacattacttgcacatgcaattgctggggaacttgacctgccaattttgaaagtggctgctccagagattgtgtctggagtatccggagagtctgagcagaagctgagagaactatttgagcaagctgtgtcaaatgcaccatgtatcattttcattgatgaaattgatgctattacccccaaaagagaagtggcttcaaaagatatggaacgaagaattgtagcccaactcctaacctgcatggatgatctgaataatgtggctgctacagcccgggtcctagttattggagctactaatcgaccagactcgttagaccctgctttgagacgtgcgggaaggttcgaccgagaaatatgcctaggtatcccagatgaagcatccagggaaagaatacttcaaacattgtgcagaaaactgaggcttcctcaagcttttgatttctgtcacttagcacacctaactccaggctttgttggtgctgatctcatggcactgtgccgagaggcagcaatgtgtgcagtcaatagagtcttaatgaagctacaggaacagcagaagaaaaatcctgaaatggaagatttgccatctaaaggagtccaggaggaaaggctgggaactgagcccacttctgaaacacaggatgaattacaaaggctgctggggttgctaagagaccaagatcccctctcagaggagcagatgcaaggactgtgcattgaactgaatgatttcattgttgctctataatgactcgatgagaccata

SEQ ID NO: 42 - DNA, 33 bp, Artificial Sequence
  sense NVL-F4
ataggtctcacatgctgtgcattgaactgaatg

SEQ ID NO: 43 - DNA, 40 bp, Artificial Sequence
  asense NVL-F4
tatggtctcatcgagtcattaccggctgagggactcctgc

SEQ ID NO: 44 - DNA, 959 bp, Artificial Sequence
  NVL-PCR-fragment 549-856
ataggtctcacatgctgtgcattgaactgaatgatttcattgttgctctatcctcagtccaaccctctgccaaaagggaaggctttgtcactgtccctaatgtgacatgggcagatattggtgccctggaagacattagagaggagctcaccatggcaatattggcaccagtacgcaacccagaccagttcaaagctcttggattggtgactccagctggggtcctccttgctggtcctcctggctgtgggaagactctgctggcgaaggctgttgcaaatgagtccggactaaattttatatctgtcaagggccccgaattactaaacatgtatgttggtgagagtgaacgtgctgtgcgacaagtttttcaacgagccaagaactcagcaccctgtgtgatattctttgatgaagtggatgctttatgtcctcgaagatcagaccgagagacaggggcaagtgtccgagtggtgaatcagctacttacagagatggatggtctggaagcacgccagcaggtttttattatggcagccactaacaggccagatataattgaccctgcaatcctgcgcccgggccgcctggacaaaacactgtttgtgggtttaccgccccctgcagatcgccttgccatcttaaaaactatcacaaaaaatggtaccaaaccaccactggatgcagatgtaaatttggaagcaattgctggtgaccttcgctgtgattgctatacgggcgcagatctctctgctttggtacgagaagcttctatctgtgccctgagacaggaaatggcaagacagaagagtggaaatgaaaaaggtgaactcaaggttagtcataagcattttgaagaagctttcaagaaagtaagatcatctatatcaaaaaaggatcaaatcatgtatgaacgtttgcaggagtccctcagccggtaatgactcgatgagaccata

SEQ ID NO: 45 - DNA, 5605 bp, Artificial Sequence
  pET24d-N-NVL[human](aa1-117)
ggggaattgtgagcggataacaattcccctctagaaataattttgtttaactttaagaaggagatataccatgagccatcatcatcatcatcatcatcattccatgaagccacgtcctgcaggatttgtggacaacaaactcaagcaacgcgttattcagtatctgaccagtaacaagtgcggcaaatacgtagacataggggttttggcgtctgatctgcagagggtgtactctatcgactatggtcggaggaaacggaatgcctttcgaattcaggtcgaaaaggtgttctccatcatcagctcagagaaagagctgaagaatctcactgaacttgaggacgaacatctcgccaaaagagccagacaaggcgaagaggacaatgagtacacagagtcctacagtgatgatgactcatcaatggaggactatcctgatccccagtcagctaactaatgactcgagtgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactata

SEQ ID NO: 46 - DNA, 5818 bp, Artificial Sequence
  pET24d-N-NVL[human](aa106-296)
ggggaattgtgagcggataacaattcccctctagaaataattttgtttaactttaagaaggagatataccatgagccatcatcatcatcatcatcatcattccatgtcaatggaggactatcctgatccccagtcagctaaccacatgaactcttctctgcttagcctgtataggaaggggaatcccgatagtgtgagcaacacaccggaaatggagcagagagaaaccaccagttccactccacgcatatcctccaaaaccggtagcattcccttgaaaactcctgctaaagactctgaaggaggctggtttatcgacaaaacgcctagcgtcaagaaggatagcttcttcctggatctgagctgtgagaagtccaatcccaagaagccaatcacagagattcaggattcaaaagattcttctcttttggagagtgatatgaaacggaaaggcaagctaaagaataaaggaagcaaaaggaagaaagaagatcttcaggaagtagatggagaaattgaagctgtcctacaaaagaaagctaaagccagggggttagaattccagatctccaacgtgaagtttgaagatgtgggaggcaatgatatgacattaaaagaggtctgcaagatgctcatacacatgcgtcacccggaggtgtaccaccactaatgactcgagtgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactata

SEQ ID NO: 47 - DNA, 6097 bp, Artificial Sequence
  pET24d-N-NVL[human](aa281-560)
ggggaattgtgagcggataacaattcccctctagaaataattttgtttaactttaagaaggagatataccatgagccatcatcatcatcatcatcatcattccatgctcatacacatgcgtcacccggaggtgtaccaccacctgggcgtcgtgccccctcgtggagttctccttcatggaccaccaggctgtgggaagacattacttgcacatgcaattgctggggaacttgacctgccaattttgaaagtggctgctccagagattgtgtctggagtatccggagagtctgagcagaagctgagagaactatttgagcaagctgtgtcaaatgcaccatgtatcattttcattgatgaaattgatgctattacccccaaaagagaagtggcttcaaaagatatggaacgaagaattgtagcccaactcctaacctgcatggatgatctgaataatgtggctgctacagcccgggtcctagttattggagctactaatcgaccagactcgttagaccctgctttgagacgtgcgggaaggttcgaccgagaaatatgcctaggtatcccagatgaagcatccagggaaagaatacttcaaacattgtgcagaaaactgaggcttcctcaagcttttgatttctgtcacttagcacacctaactccaggctttgttggtgctgatctcatggcactgtgccgagaggcagcaatgtgtgcagtcaatagagtcttaatgaagctacaggaacagcagaagaaaaatcctgaaatggaagatttgccatctaaaggagtccaggaggaaaggctgggaactgagcccacttctgaaacacaggatgaattacaaaggctgctggggttgctaagagaccaagatcccctctcagaggagcagatgcaaggactgtgcattgaactgaatgatttcattgttgctctataatgactcgagtgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactata

SEQ ID NO: 48 - DNA, 6181 bp, Artificial Sequence
  pET24d-N-NVL[human](aa549-856)
ggggaattgtgagcggataacaattcccctctagaaataattttgtttaactttaagaaggagatataccatgagccatcatcatcatcatcatcatcattccatgctgtgcattgaactgaatgatttcattgttgctctatcctcagtccaaccctctgccaaaagggaaggctttgtcactgtccctaatgtgacatgggcagatattggtgccctggaagacattagagaggagctcaccatggcaatattggcaccagtacgcaacccagaccagttcaaagctcttggattggtgactccagctggggtcctccttgctggtcctcctggctgtgggaagactctgctggcgaaggctgttgcaaatgagtccggactaaattttatatctgtcaagggccccgaattactaaacatgtatgttggtgagagtgaacgtgctgtgcgacaagtttttcaacgagccaagaactcagcaccctgtgtgatattctttgatgaagtggatgctttatgtcctcgaagatcagaccgagagacaggggcaagtgtccgagtggtgaatcagctacttacagagatggatggtctggaagcacgccagcaggtttttattatggcagccactaacaggccagatataattgaccctgcaatcctgcgcccgggccgcctggacaaaacactgtttgtgggtttaccgccccctgcagatcgccttgccatcttaaaaactatcacaaaaaatggtaccaaaccaccactggatgcagatgtaaatttggaagcaattgctggtgaccttcgctgtgattgctatacgggcgcagatctctctgctttggtacgagaagcttctatctgtgccctgagacaggaaatggcaagacagaagagtggaaatgaaaaaggtgaactcaaggttagtcataagcattttgaagaagctttcaagaaagtaagatcatctatatcaaaaaaggatcaaatcatgtatgaacgtttgcaggagtccctcagccggtaatgactcgagtgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactata

SEQ ID NO: 49 - protein, 128 aa, Artificial Sequence
  H8-NVL[human](aa1-117)
MSHHHHHHHHSMKPRPAGFVDNKLKQRVIQYLTSNKCGKYVDIGVLASDLQRVYSIDYGRRKRNAFRIQVEKVFSIISSEKELKNLTELEDEHLAKRARQGEEDNEYTESYSDDDSSMEDYPDPQSAN

SEQ ID NO: 50 - protein, 199 aa, Artificial Sequence
  H8-NVL[human](aa106-292)
MSHHHHHHHHSMSMEDYPDPQSANHMNSSLLSLYRKGNPDSVSNTPEMEQRETTSSTPRISSKTGSIPLKTPAKDSEGGWFIDKTPSVKKDSFFLDLSCEKSNPKKPITEIQDSKDSSLLESDMKRKGKLKNKGSKRKKEDLQEVDGEIEAVLQKKAKARGLEFQISNVKFEDVGGNDMTLKEVCKMLIHMRHPEVYHH

SEQ ID NO: 51 - protein, 292 aa, Artificial Sequence
  H8-NVL[human](aa281-560)
MSHHHHHHHHSMLIHMRHPEVYHHLGVVPPRGVLLHGPPGCGKTLLAHAIAGELDLPILKVAAPEIVSGVSGESEQKLRELFEQAVSNAPCIIFIDEIDAITPKREVASKDMERRIVAQLLTCMDDLNNVAATARVLVIGATNRPDSLDPALRRAGRFDREICLGIPDEASRERILQTLCRKLRLPQAFDFCHLAHLTPGFVGADLMALCREAAMCAVNRVLMKLQEQQKKNPEMEDLPSKGVQEERLGTEPTSETQDELQRLLGLLRDQDPLSEEQMQGLCIELNDFIVAL

SEQ ID NO: 52 - protein, 320 aa, Artificial Sequence
  H8-NVL[human](aa549-856)
MSHHHHHHHHSMLCIELNDFIVALSSVQPSAKREGFVTVPNVTWADIGALEDIREELTMAILAPVRNPDQFKALGLVTPAGVLLAGPPGCGKTLLAKAVANESGLNFISVKGPELLNMYVGESERAVRQVFQRAKNSAPCVIFFDEVDALCPRRSDRETGASVRVVNQLLTEMDGLEARQQVFIMAATNRPDIIDPAILRPGRLDKTLFVGLPPPADRLAILKTITKNGTKPPLDADVNLEAIAGDLRCDCYTGADLSALVREASICALRQEMARQKSGNEKGELKVSHKHFEEAFKKVRSSISKKDQIMYERLQESLSR

SEQ ID NO: 53 - DNA, 36 bp, Artificial Sequence
  sense CD2BP2-F1
atacgtctcgcatgccaaagaggaaagtgaccttcc

SEQ ID NO: 54 - DNA, 44 bp, Artificial Sequence
  asense CD2BP2-F1
tatcgtctcgtcgatcagatcttcacccagtcaatgttgtccag

SEQ ID NO: 55 - DNA, 412 bp, Artificial Sequence
  CD2BP2-PCR-fragment 1-128
atacgtctcgcatgccaaagaggaaagtgaccttccaaggcgtgggagatgaggaggatgaggatgaaatcattgtccccaagaagaagctggtggaccctgtggctgggtcagggggtcctgggagccgctttaaaggcaaacactctttggatagcgatgaggaggaggatgatgatgatggggggtccagcaaatatgacatcttggcctcagaggatgtagaaggtcaggaggcagccacactccccagcgaggggggtgttcggatcacaccctttaacctgcaggaggagatggaggaaggccactttgatgccgatggcaactacttcctgaaccgggatgctcagatccgagacagctggctggacaacattgactgggtgaagatctgatcgacgagacgata

SEQ ID NO: 56 - DNA, 40 bp, Artificial Sequence
  sense CD2BP2-F2
atacgtctcgcatggacagctggctggacaacattgactg

SEQ ID NO: 57 - DNA, 43 bp, Artificial Sequence
  asense CD2BP2-F2
tatcgtctcgtcgatcacagttcctcctccgccaactcctcag

SEQ ID NO: 58 - DNA, 463 bp, Artificial Sequence
  CD2BP2-PCR-fragment 117-260
atacgtctcgcatggacagctggctggacaacattgactgggtgaagatccgggagcggccacctggccagcgccaggcctcagactcggaggaggaggacagcttgggccagacctcaatgagtgcccaagccctcttggagggacttttggagctcctattgcctagagagacagtggctggggcactgaggcgtctgggggcccgaggaggaggcaaagggagaaaggggcctgggcaacccagttcccctcagcgcctggaccggctctccgggttggccgaccagatggtggcccggggcaaccttggtgtgtaccaggaaacaagggaacggttggctatgcgtctgaagggtttggggtgtcagaccctaggaccccacaatcccacacccccaccctccctggacatgttcgctgaggagttggcggaggaggaactgtgatcgacgagacgata

SEQ ID NO: 59 - DNA, 41 bp, Artificial Sequence
  sense CD2BP2-F3
atacgtctcgcatggacatgttcgctgaggagttggcggag

SEQ ID NO: 60 - DNA, 43 bp, Artificial Sequence
  asense CD2BP2-F3
tatcgtctcgtcgatcatcaggtgtagaggtcaaagtcaatgc

SEQ ID NO: 61 - DNA, 313 bp, Artificial Sequence
  CD2BP2-PCR-fragment 249-341
atacgtctcgcatggacatgttcgctgaggagttggcggaggaggaactggagaccccaacccctacccagagaggagaagcagagtcgcggggagatggtctggtggatgtgatgtgggaatataagtgggagaacacgggggatgccgagctgtatgggcccttcaccagcgcccagatgcagacctgggtgagtgaaggctacttcccggacggtgtttattgccggaagctggacccccctggtggtcagttctacaactccaaacgcattgactttgacctctacacctgatgatcgacgagacgata

SEQ ID NO: 62 - DNA, 5634 bp, Artificial Sequence
  pET24d-N-CD2BP2[human](aa1-128)
ggggaattgtgagcggataacaattcccctctagaaataattttgtttaactttaagaaggagatataccatgagccatcatcatcatcatcatcatcattccatgccaaagaggaaagtgaccttccaaggcgtgggagatgaggaggatgaggatgaaatcattgtccccaagaagaagctggtggaccctgtggctgggtcagggggtcctgggagccgctttaaaggcaaacactctttggatagcgatgaggaggaggatgatgatgatggggggtccagcaaatatgacatcttggcctcagaggatgtagaaggtcaggaggcagccacactccccagcgaggggggtgttcggatcacaccctttaacctgcaggaggagatggaggaaggccactttgatgccgatggcaactacttcctgaaccgggatgctcagatccgagacagctggctggacaacattgactgggtgaagatctgatcgagtgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactata

SEQ ID NO: 63 - DNA, 5685 bp, Artificial Sequence
  pET24d-N-CD2BP2[human](aa117-260)
ggggaattgtgagcggataacaattcccctctagaaataattttgtttaactttaagaaggagatataccatgagccatcatcatcatcatcatcatcattccatggacagctggctggacaacattgactgggtgaagatccgggagcggccacctggccagcgccaggcctcagactcggaggaggaggacagcttgggccagacctcaatgagtgcccaagccctcttggagggacttttggagctcctattgcctagagagacagtggctggggcactgaggcgtctgggggcccgaggaggaggcaaagggagaaaggggcctgggcaacccagttcccctcagcgcctggaccggctctccgggttggccgaccagatggtggcccggggcaaccttggtgtgtaccaggaaacaagggaacggttggctatgcgtctgaagggtttggggtgtcagaccctaggaccccacaatcccacacccccaccctccctggacatgttcgctgaggagttggcggaggaggaactgtgatcgagtgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactata

SEQ ID NO: 64 - DNA, 5535 bp, Artificial Sequence
  pET24d-N-CD2BP2[human](aa249-341)
ggggaattgtgagcggataacaattcccctctagaaataattttgtttaactttaagaaggagatataccatgagccatcatcatcatcatcatcatcattccatggacatgttcgctgaggagttggcggaggaggaactggagaccccaacccctacccagagaggagaagcagagtcgcggggagatggtctggtggatgtgatgtgggaatataagtgggagaacacgggggatgccgagctgtatgggcccttcaccagcgcccagatgcagacctgggtgagtgaaggctacttcccggacggtgtttattgccggaagctggacccccctggtggtcagttctacaactccaaacgcattgactttgacctctacacctgatgatcgagtgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactata

SEQ ID NO: 65 - protein, 139 aa, Artificial Sequence
  H8-CD2BP2[human](aa1-128)
MSHHHHHHHHSMPKRKVTFQGVGDEEDEDEIIVPKKKLVDPVAGSGGPGSRFKGKHSLDSDEEEDDDDGGSSKYDILASEDVEGQEAATLPSEGGVRITPFNLQEEMEEGHFDADGNYFLNRDAQIRDSWLDNIDWVKI

SEQ ID NO: 66 - protein, 156 aa, Artificial Sequence
  H8-CD2BP2[human](aa117-260)
MSHHHHHHHHSMDSWLDNIDWVKIRERPPGQRQASDSEEEDSLGQTSMSAQALLEGLLELLLPRETVAGALRRLGARGGGKGRKGPGQPSSPQRLDRLSGLADQMVARGNLGVYQETRERLAMRLKGLGCQTLGPHNPTPPPSLDMFAEELAEEEL

SEQ ID NO: 67 - protein, 105 aa, Artificial Sequence
  H8-CD2BP2[human](aa249-341)
MSHHHHHHHHSMDMFAEELAEEELETPTPTQRGEAESRGDGLVDVMWEYKWENTGDAELYGPFTSAQMQTWVSEGYFPDGVYCRKLDPPGGQFYNSKRIDFDLYT

SEQ ID NO: 68 - DNA, 33 bp, Artificial Sequence; identical to SEQ ID NO: 25
  sense TJP1-F1/2

SEQ ID NO: 69 - DNA, 43 bp, Artificial Sequence
  asense TJP1-F1/4
ataggtctcctcgagttaagggatgatgcctcgttctacctcc

SEQ ID NO: 70 - DNA, 1754 bp, Artificial Sequence
  TJP1-PCR-fragment 1-575
ataggtctctcatgtccgccagagctgcggctgccaagagcacagcaatggaggaaacagctatatgggaacaacatacagtgacgcttcacagggctcctggatttggatttggaattgcaatatctggtggacgagataatcctcattttcagagtggggaaacgtcaatagtgatttcagatgtgctgaaaggaggaccagctgaaggacagctacaggaaaatgaccgagttgcaatggttaacggagtttcaatggataatgttgaacatgcttttgctgttcagcaactaaggaaaagtgggaaaaatgcaaaaattacaattagaaggaagaagaaagttcaaataccagtaagtcgtcctgatcctgaaccagtatctgataatgaagaagatagttatgatgaggaaatacatgatccaagaagtggccggagtggtgtggttaacagaaggagtgagaagatttggccgagggatagaagtgcaagtagagagaggagcttgtccccgcggtcagacaggcggtcagtggcttccagccagcctgctaaacctactaaagtcacactggtgaaatcccggaaaaatgaagaatatggtcttcgattggcaagccatatatttgttaaggaaatttcacaagatagtttggcagcaagagatggcaatattcaagaaggtgatgttgtattgaagataaatggtactgtgacagaaaatatgtcattgacagatgcaaagacattgatagaaaggtctaaaggcaaattaaaaatggtagttcaaagagatgaacgggctacgctattgaatgtccctgatctttctgacagcatccactctgctaatgcctctgagagagacgacatttcagaaattcagtcactggcatcagatcattctggtcgatcacacgataggcctccccgccgcagccggtcacgatctcctgaccagcggtcagagccttctgatcattccaggcactcgccgcagcagccaagcaatggcagtctccggagtagagatgaagagagaatttctaaacctggggctgtctcaactcctgtaaagcatgctgatgatcacacacctaaaacagtggaagaagttacagttgaaagaaatgagaaacaaacaccttctcttccagaaccaaagcctgtgtatgcccaagttgggcaaccagatgtggatttacctgtcagtccatctgatggtgtcctacctaattcaactcatgaagatgggattcttcggcccagcatgaaattggtaaaattcagaaaaggagatagtgtgggtttgcggctggctggtggaaatgatgttggaatatttgtagctggcgttctagaagatagccctgcagccaaggaaggcttagaggaaggtgatcaaattctcagggtaaacaacgtagattttacaaatatcataagagaagaagccgtccttttcctgcttgacctccctaaaggagaagaagtgaccatattggctcagaagaagaaggatgtttatcgtcgcattgtagaatcagatgtaggagattctttctatattagaacccattttgaatatgaaaaggaatctccctatggacttagttttaacaaaggagaggtgttccgtgttgtggataccttgtacaatggaaaactgggctcttggcttgctattcgaattggtaaaaatcataaggaggtagaacgaggcatcatcccttaactcgaggagacctat

SEQ ID NO: 71 - DNA, 43 bp, Artificial Sequence
  asense TJP1-F2
ataggtctcctcgagttacggccaaatcttctcactccttctg

SEQ ID NO: 72 - DNA, 497 bp, Artificial Sequence
  TJP1-PCR-fragment 1-156
ataggtctctcatgtccgccagagctgcggccgccaagagcacagcaatggaggaaacagctatatgggaacaacatacagtgacgcttcacagggctcctggatttggatttggaattgcaatatctggtggacgagataatcctcattttcagagtggggaaacgtcaatagtgatttcagatgtgctgaaaggaggaccagctgaaggacagctacaggaaaatgaccgagttgcaatggttaacggagtttcaatggataatgttgaacatgcttttgctgttcagcaactaaggaaaagtgggaaaaatgcaaaaattacaattagaaggaagaagaaagttcaaataccagtaagtcgtcctgatcctgaaccagtatctgataatgaagaagatagttatgatgaggaaatacatgatccaagaagtggccggagtggtgtggttaacagaaggagtgagaagatttggccgtaactcgaggagacctat

SEQ ID NO: 73 - DNA, 39 bp, Artificial Sequence
  sense TJP1-F3
ataggtctcacatgagtggtgtggttaacagaaggagtg

SEQ ID NO: 74 - DNA, 42 bp, Artificial Sequence
  asense TJP1-F3
ataggtctcctcgagttacctatcgtgtgatcgaccagaatg

SEQ ID NO: 75 - DNA, 521 bp, Artificial Sequence
  TJP1-PCR-fragment 144-306
ataggtctcacatgagtggtgtggttaacagaaggagtgagaagatttggccgagggatagaagtgcaagtagagagaggagcttgtccccgcggtcagacaggcggtcagtggcttccagccagcctgctaaacctactaaagtcacactggtgaaatcccggaaaaatgaagaatatggtcttcgattggcaagccatatatttgttaaggaaatttcacaagatagtttggcagcaagagatggcaatattcaagaaggtgatgttgtattgaagataaatggtactgtgacagaaaatatgtcattgacagatgcaaagacattgatagaaaggtctaaaggcaaattaaaaatggtagttcaaagagatgaacgggctacgctattgaatgtccctgatctttctgacagcatccactctgctaatgcctctgagagagacgacatttcagaaattcagtcactggcatcagatcattctggtcgatcacacgataggtaactcgaggagacctat

SEQ ID NO: 76 - DNA, 39 bp, Artificial Sequence
  sense TJP1-F4
ataggtctctcatgtcactggcatcagatcattctggtc

SEQ ID NO: 77 - DNA, 878 bp, Artificial Sequence
  TJP1-PCR-fragment 294-575
ataggtctctcatgtcactggcatcagatcattctggtcgatcacacgataggcctccccgccgcagccggtcacgatctcctgaccagcggtcagagccttctgatcattccaggcactcgccgcagcagccaagcaatggcagtctccggagtagagatgaagagagaatttctaaacctggggctgtctcaactcctgtaaagcatgctgatgatcacacacctaaaacagtggaagaagttacagttgaaagaaatgagaaacaaacaccttctcttccagaaccaaagcctgtgtatgcccaagttgggcaaccagatgtggatttacctgtcagtccatctgatggtgtcctacctaattcaactcatgaagatgggattcttcggcccagcatgaaattggtaaaattcagaaaaggagatagtgtgggtttgcggctggctggtggaaatgatgttggaatatttgtagctggcgttctagaagatagccctgcagccaaggaaggcttagaggaaggtgatcaaattctcagggtaaacaacgtagattttacaaatatcataagagaagaagccgtccttttcctgcttgacctccctaaaggagaagaagtgaccatattggctcagaagaagaaggatgtttatcgtcgcattgtagaatcagatgtaggagattctttctatattagaacccattttgaatatgaaaaggaatctccctatggacttagttttaacaaaggagaggtgttccgtgttgtggataccttgtacaatggaaaactgggctcttggcttgctattcgaattggtaaaaatcataaggaggtagaacgaggcatcatcccttaactcgaggagacctat

SEQ ID NO: 78 - DNA, 6976 bp, Artificial Sequence
  pET24d-N-TJP1[human](aa1-575)
ggggaattgtgagcggataacaattcccctctagaaataattttgtttaactttaagaaggagatataccatgagccatcatcatcatcatcatcatcattccatgtccgccagagctgcggctgccaagagcacagcaatggaggaaacagctatatgggaacaacatacagtgacgcttcacagggctcctggatttggatttggaattgcaatatctggtggacgagataatcctcattttcagagtggggaaacgtcaatagtgatttcagatgtgctgaaaggaggaccagctgaaggacagctacaggaaaatgaccgagttgcaatggttaacggagtttcaatggataatgttgaacatgcttttgctgttcagcaactaaggaaaagtgggaaaaatgcaaaaattacaattagaaggaagaagaaagttcaaataccagtaagtcgtcctgatcctgaaccagtatctgataatgaagaagatagttatgatgaggaaatacatgatccaagaagtggccggagtggtgtggttaacagaaggagtgagaagatttggccgagggatagaagtgcaagtagagagaggagcttgtccccgcggtcagacaggcggtcagtggcttccagccagcctgctaaacctactaaagtcacactggtgaaatcccggaaaaatgaagaatatggtcttcgattggcaagccatatatttgttaaggaaatttcacaagatagtttggcagcaagagatggcaatattcaagaaggtgatgttgtattgaagataaatggtactgtgacagaaaatatgtcattgacagatgcaaagacattgatagaaaggtctaaaggcaaattaaaaatggtagttcaaagagatgaacgggctacgctattgaatgtccctgatctttctgacagcatccactctgctaatgcctctgagagagacgacatttcagaaattcagtcactggcatcagatcattctggtcgatcacacgataggcctccccgccgcagccggtcacgatctcctgaccagcggtcagagccttctgatcattccaggcactcgccgcagcagccaagcaatggcagtctccggagtagagatgaagagagaatttctaaacctggggctgtctcaactcctgtaaagcatgctgatgatcacacacctaaaacagtggaagaagttacagttgaaagaaatgagaaacaaacaccttctcttccagaaccaaagcctgtgtatgcccaagttgggcaaccagatgtggatttacctgtcagtccatctgatggtgtcctacctaattcaactcatgaagatgggattcttcggcccagcatgaaattggtaaaattcagaaaaggagatagtgtgggtttgcggctggctggtggaaatgatgttggaatatttgtagctggcgttctagaagatagccctgcagccaaggaaggcttagaggaaggtgatcaaattctcagggtaaacaacgtagattttacaaatatcataagagaagaagccgtccttttcctgcttgacctccctaaaggagaagaagtgaccatattggctcagaagaagaaggatgtttatcgtcgcattgtagaatcagatgtaggagattctttctatattagaacccattttgaatatgaaaaggaatctccctatggacttagttttaacaaaggagaggtgttccgtgttgtggataccttgtacaatggaaaactgggctcttggcttgctattcgaattggtaaaaatcataaggaggtagaacgaggcatcatcccttaactcgagtgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactata

SEQ ID NO: 79 - DNA, 5719 bp, Artificial Sequence
  pET24d-N-TJP1[human](aa1-156)
ggggaattgtgagcggataacaattcccctctagaaataattttgtttaactttaagaaggagatataccatgagccatcatcatcatcatcatcatcattccatgtccgccagagctgcggccgccaagagcacagcaatggaggaaacagctatatgggaacaacatacagtgacgcttcacagggctcctggatttggatttggaattgcaatatctggtggacgagataatcctcattttcagagtggggaaacgtcaatagtgatttcagatgtgctgaaaggaggaccagctgaaggacagctacaggaaaatgaccgagttgcaatggttaacggagtttcaatggataatgttgaacatgcttttgctgttcagcaactaaggaaaagtgggaaaaatgcaaaaattacaattagaaggaagaagaaagttcaaataccagtaagtcgtcctgatcctgaaccagtatctgataatgaagaagatagttatgatgaggaaatacatgatccaagaagtggccggagtggtgtggttaacagaaggagtgagaagatttggccgtaactcgagtgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactata

SEQ ID NO: 80 - DNA, 5743 bp, Artificial Sequence
  pET24d-N-TJP1[human](aa144-306)
ggggaattgtgagcggataacaattcccctctagaaataattttgtttaactttaagaaggagatataccatgagccatcatcatcatcatcatcatcattccatgagtggtgtggttaacagaaggagtgagaagatttggccgagggatagaagtgcaagtagagagaggagcttgtccccgcggtcagacaggcggtcagtggcttccagccagcctgctaaacctactaaagtcacactggtgaaatcccggaaaaatgaagaatatggtcttcgattggcaagccatatatttgttaaggaaatttcacaagatagtttggcagcaagagatggcaatattcaagaaggtgatgttgtattgaagataaatggtactgtgacagaaaatatgtcattgacagatgcaaagacattgatagaaaggtctaaaggcaaattaaaaatggtagttcaaagagatgaacgggctacgctattgaatgtccctgatctttctgacagcatccactctgctaatgcctctgagagagacgacatttcagaaattcagtcactggcatcagatcattctggtcgatcacacgataggtaactcgagtgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactata

SEQ ID NO: 81 - DNA, 6100 bp, Artificial Sequence
  pET24d-N-TJP1[human](aa294-575)
ggggaattgtgagcggataacaattcccctctagaaataattttgtttaactttaagaaggagatataccatgagccatcatcatcatcatcatcatcattccatgtcactggcatcagatcattctggtcgatcacacgataggcctccccgccgcagccggtcacgatctcctgaccagcggtcagagccttctgatcattccaggcactcgccgcagcagccaagcaatggcagtctccggagtagagatgaagagagaatttctaaacctggggctgtctcaactcctgtaaagcatgctgatgatcacacacctaaaacagtggaagaagttacagttgaaagaaatgagaaacaaacaccttctcttccagaaccaaagcctgtgtatgcccaagttgggcaaccagatgtggatttacctgtcagtccatctgatggtgtcctacctaattcaactcatgaagatgggattcttcggcccagcatgaaattggtaaaattcagaaaaggagatagtgtgggtttgcggctggctggtggaaatgatgttggaatatttgtagctggcgttctagaagatagccctgcagccaaggaaggcttagaggaaggtgatcaaattctcagggtaaacaacgtagattttacaaatatcataagagaagaagccgtccttttcctgcttgacctccctaaaggagaagaagtgaccatattggctcagaagaagaaggatgtttatcgtcgcattgtagaatcagatgtaggagattctttctatattagaacccattttgaatatgaaaaggaatctccctatggacttagttttaacaaaggagaggtgttccgtgttgtggataccttgtacaatggaaaactgggctcttggcttgctattcgaattggtaaaaatcataaggaggtagaacgaggcatcatcccttaactcgagtgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactata

SEQ ID NO: 82 - protein, 586 aa, Artificial Sequence
  H8-TJP1[human](aa1-575)
MSHHHHHHHHSMSARAAAAKSTAMEETAIWEQHTVTLHRAPGFGFGIAISGGRDNPHFQSGETSIVISDVLKGGPAEGQLQENDRVAMVNGVSMDNVEHAFAVQQLRKSGKNAKITIRRKKKVQIPVSRPDPEPVSDNEEDSYDEEIHDPRSGRSGVVNRRSEKIWPRDRSASRERSLSPRSDRRSVASSQPAKPTKVTLVKSRKNEEYGLRLASHIFVKEISQDSLAARDGNIQEGDVVLKINGTVTENMSLTDAKTLIERSKGKLKMVVQRDERATLLNVPDLSDSIHSANASERDDISEIQSLASDHSGRSHDRPPRRSRSRSPDQRSEPSDHSRHSPQQPSNGSLRSRDEERISKPGAVSTPVKHADDHTPKTVEEVTVERNEKQTPSLPEPKPVYAQVGQPDVDLPVSPSDGVLPNSTHEDGILRPSMKLVKFRKGDSVGLRLAGGNDVGIFVAGVLEDSPAAKEGLEEGDQILRVNNVDFTNIIREEAVLFLLDLPKGEEVTILAQKKKDVYRRIVESDVGDSFYIRTHFEYEKESPYGLSFNKGEVFRVVDTLYNGKLGSWLAIRIGKNHKEVERGIIP

SEQ ID NO: 83 - protein, 167 aa, Artificial Sequence
  H8-TJP1[human](aa1-156)
MSHHHHHHHHSMSARAAAAKSTAMEETAIWEQHTVTLHRAPGFGFGIAISGGRDNPHFQSGETSIVISDVLKGGPAEGQLQENDRVAMVNGVSMDNVEHAFAVQQLRKSGKNAKITIRRKKKVQIPVSRPDPEPVSDNEEDSYDEEIHDPRSGRSGVVNRRSEKIWP

SEQ ID NO: 84 - protein, 175 aa, Artificial Sequence
  H8-TJP1[human](aa144-306)
MSHHHHHHHHSMSGVVNRRSEKIWPRDRSASRERSLSPRSDRRSVASSQPAKPTKVTLVKSRKNEEYGLRLASHIFVKEISQDSLAARDGNIQEGDVVLKINGTVTENMSLTDAKTLIERSKGKLKMVVQRDERATLLNVPDLSDSIHSANASERDDISEIQSLASDHSGRSHDR

SEQ ID NO: 85 - protein, 294 aa, Artificial Sequence
  H8-TJP1[human](aa294-575)
MSHHHHHHHHSMSLASDHSGRSHDRPPRRSRSRSPDQRSEPSDHSRHSPQQPSNGSLRSRDEERISKPGAVSTPVKHADDHTPKTVEEVTVERNEKQTPSLPEPKPVYAQVGQPDVDLPVSPSDGVLPNSTHEDGILRPSMKLVKFRKGDSVGLRLAGGNDVGIFVAGVLEDSPAAKEGLEEGDQILRVNNVDFTNIIREEAVLFLLDLPKGEEVTILAQKKKDVYRRIVESDVGDSFYIRTHFEYEKESPYGLSFNKGEVFRVVDTLYNGKLGSWLAIRIGKNHKEVERGIIP

SEQ ID NO: 86 - protein, 13 aa, Artificial Sequence
  NVL-fragment(aa591-603)
EELTMAILAPVRN

SEQ ID NO: 87 - protein, 11 aa, Artificial Sequence
  NVL-fragment(aa607-617)
FKALGLVTPAG

SEQ ID NO: 88 - protein, 11 aa, Artificial Sequence
  NVL-fragment(aa685-695)
ALCPRRSDRET

SEQ ID NO: 89 - protein, 12 aa, Artificial Sequence
  NVL-fragment(aa742-753)
KTLFVGLPPPAD

SEQ ID NO: 90 - protein, 12 aa, Artificial Sequence
  NVL-fragment(aa761-772)
ITKNGTKPPLDA

SEQ ID NO: 91 - protein, 12 aa, Artificial Sequence
  NVL-fragment(aa778-789)
AIAGDLRCDCYT

SEQ ID NO: 92 - protein, 18 aa, Artificial Sequence
  NVL-fragment(aa809-826)
MARQKSGNEKGELKVSHK

SEQ ID NO: 93 - protein, 13 aa, Artificial Sequence
  NVL-fragment(aa844-856)
QIMYERLQESLSR

(19) United States
(12) Patent Application Publication
(10) Pub. No.: US 2022/0317121 A1
(43) Pub. Date: Oct. 6, 2022

(54) Method and means for detecting an autoantibody

(71) Applicant: EUROIMMUN Medizinische Labordiagnostika AG, Luebeck (DE)

(72) Inventors: Zitao ZENG, Luebeck (DE); Lars Komorowski, Ratzeburg (DE); Ramona Miske, Luebeck (DE); Madeleine Scharf, Selmsdorf (DE); Yvonne Denno, Luebeck (DE); Christiane Radzimski, Reinfeld (DE); Nadine Rochow, Selmsdorf (DE); Stefanie Brakopp, Wittenburg OT Lehsen (DE)

(73) Assignee: EUROIMMUN Medizinische Labordiagnostika AG, Luebeck (DE)

(21) Appl. No.: 17/701,779

(22) Filed: Mar. 23, 2022

(30) Foreign Application Priority Data
Mar. 25, 2021 (EP) 21165011.4

(51) Int. Cl. G01N 33/564; G01N 33/68

(52) U.S. Cl. CPC G01N 33/564; G01N 33/6854; G01N 2800/24

(57) ABSTRACT

A method can be used for detecting, in a sample, an autoantibody binding to a polypeptide having the sequence SEQ ID NO: 1, SEQ ID NO: 11 or SEQ ID NO: 24. A carrier containing said polypeptide and an autoantibody binding to said polypeptide are useful. The autoantibody may be used for the diagnosis of a disease, and a method can be used for isolating an autoantibody binding to said polypeptide. The polypeptide and a kit containing said polypeptide are useful, and can be used for the manufacture of a kit or medical device. A method involves contacting a medical or diagnostic device containing said polypeptide with a buffered solution containing an antibody binding specifically to said polypeptide. A sample containing said autoantibody as a positive control and a diluted sample containing said autoantibody are useful.

### CROSS-REFERENCE TO RELATED APPLICATIONS

This application claims priority to European Application No. 21165011.4, filed on Mar. 25, 2021, the content of which is hereby incorporated by reference in its entirety.

### REFERENCE TO A SEQUENCE LISTING

The present application is accompanied by an ASCII text file as a computer readable form containing the sequence listing entitled, “003941US_SL_ST25.txt”, created on Mar. 10, 2022, with a file size of 263,255 bytes, the content of which is hereby incorporated by reference in its entirety.

### BACKGROUND OF THE INVENTION

### Field of the Invention

The present invention relates to a method for detecting in a sample an autoantibody binding to a polypeptide having the sequence SEQ ID NO: 1, SEQ ID NO: 11 or SEQ ID NO: 24, a carrier comprising said polypeptide, an autoantibody binding said polypeptide, a use of the autoantibody for the diagnosis of a disease, a method for isolating an autoantibody binding to said polypeptide, a kit comprising said polypeptide, a use of said polypeptide, said carrier or said autoantibody for the manufacture of a kit or medical device, a method comprising the step contacting a medical or diagnostic device comprising said polypeptide with a buffered solution comprising an antibody binding specifically to said polypeptide, a use of a sample comprising said autoantibody as a positive control and a diluted sample comprising said autoantibody.

### Description of Related Art

Autoimmune diseases are understood to mean diseases in which the immune system is directed against intact structures endogenous to the body. In many autoimmune diseases, the body produces detectable autoantibodies which can bind against proteins endogenous to the body and thus cause disorders. In other cases, the presence of autoantibodies does not appear to be causally responsible for the outbreak of a disease.

ANAs (antinuclear autoantibodies) are a group of autoantibodies that targets the nuclear components of mammalian cells. They are mainly associated with systemic autoimmune rheumatic diseases (SARD) and some of the ANAs are biomarkers for certain SARDs. For example, anti-double-strand DNA (dsDNA), anti-Sm and anti-ribosomal P are tightly associated with systemic lupus erythematosus (SLE), while anti-Scl70, anti-centromere and anti-RNA polymerase III are biomarkers for systemic sclerosis (SSc). Meanwhile, increasing evidence shows that elevated level of ANAs in cancer patients is significant in confronting tumor cells. For example, multiple studies revealed that anti-dsDNA autoantibodies can induce the apoptosis of cancer cells and inhibit the growth of tumors. Some well-established ANAs for SARD are found to be associated with different cancers, for example anti-dsDNA anti-Ro/SS-A and anti-Scl70 are presented with gastrointestinal cancer, hematological malignancies and lung cancer, respectively. Additionally, ANAs could be detected in approximately 5.9-30.8% of healthy individuals, more frequent in women than in men. Elder people show a higher rate of being ANA positive. This increases the complexity of application of ANAs in diagnostic and thus urges the identification of individual ANA and the assessment of their clinical values.

The frequency (prevalence) of anti-nuclear antibodies in inflammatory rheumatic diseases lies between 20 and 100%. Therefore, differential ANA diagnostics is indispensable for the diagnosis of individual rheumatic diseases and their differentiation from other autoimmune diseases.

The immunoassay for the detection of ANA is dated back to 1948, Hargraves et al. discovered and applied lupus erythematosus (LE) cells in order to aid the diagnosis of SLE patients, though it was found later that the LE cell test was neither sensitive nor specific enough (R. B. Conn, “Practice Parameter—The Lupus Erythematosus Cell Test: An Obsolete Test Now Superseded by Definitive Immunologic Tests,” American Journal of Clinical Pathology, pp. 65-66, 1 Jan. 1994). In the late 1950s, the indirect immunofluorescence assay (IFA) based on animal tissue substrates, such as kidney from rats, was established. The principle of the test is that the specific ANAs bound to the antigens coated on object slides are detected by fluorescence labeled anti-human antibodies, which can be visualized under a fluorescence microscope. For the better evaluation of the result, the observed positive fluorescence signals are described as different patterns, like homogenous and granular, which is useful in subclassification of the positive results (Kavanaugh et al., “Guidelines for Clinical Use of the Antinuclear Antibody Test and Tests for Specific Autoantibodies to Nuclear Antigens.” Archives of Pathology & Laboratory Medicine, vol. 124, no. 1, pp. 71-81. August 2000). Another important parameter in the evaluation of the results is the highest serum dilution, with which the patterns are still visible; this is defined as titer. Two decades later, HeLa-derived human epithelial type 2 (HEp-2) cells, which show better sensitivity and specificity, were applied in the IFA. When compared to cryosection of animal tissues, HEp-2 cells have the advantage of a large nucleus and a better visibility of other organelles, high division rate and an expression profile with more than 100 autoantigens, which contributes to easier observation and interpretation of the results. According to the guideline for ANA tests, a negative IFA result indicates a non-SARD condition while a positive result should be further differentiated with available monospecific tests.

Monospecific ANA tests were developed as the corresponding nuclear autoantigens were identified since 1960s (Tan et al., “Characteristics of a Soluble Nuclear Antigen Precipitating with Sera of Patients with Systemic Lupus Erythematosus,” J Immunol, pp. 96(3):464-471, 1 Mar. 1966). The first International Consensus Statement on standardized nomenclature of HEp-2 cell patterns in indirect immunofluorescence (ICAP, www.anapatterns.org) defined fifteen nuclear patterns and nine cytoplasmic patterns which are relevant for the diagnosis of various autoimmune diseases. Currently, different kinds of solid phase-based assays, including enzyme-linked immunosorbent assay (ELISA), line blot. Western blot and chemiluminescence immunoassay (CLIA), are available with native or recombinant antigens. These tests have a similar principle as IFA, where the secondary anti-human antibodies are labelled with enzymes or chemical compounds that can generate color-change reactions or light emission reactions (Ludwig et al., “Mechanisms of autoantibody-induced pathology,” Frontiers in Immunology, p. 8.603, 31 May 2017).

The occurrence of high titers of ANAs may be years before the development of clinical syndromes. Arbuckle and colleagues have reported that ANAs could be detected in 88% of SLE patients in a mean of 3.3 years before onset of the disease (Arbuckle et al., “Development of Autoantibodies before the Clinical Onset of Systemic Lupus Erythematosus,” N Engl J Med, pp. 349:1526-33, 2003). This demonstrates the significance of ANAs in early diagnosis of the disease and a possible specific treatment. However, the utility of ANA diagnostics differs from one to another. Some ANAs showed high disease specificity and are included in the diagnostic criteria and/or reflecting disease activity, while some others show weak association with SARD or even negative relations.

Accordingly, ANAs are important diagnostic and prognostic markers. However, regardless of more than half a century of study and experience, there are still some gaps remaining in ANA diagnostics that urge to be resolved. Nowadays, more physicians, rather than just rheumatologists, are ordering ANA tests for preventive medicine and precision health, which leads to increasing numbers of positive result of ANA tests, especially positive IFA with HEp-2 cells, with unclear clinical value. However, it is not only important to report the ANA patterns and titers on HEp-2 cells but also to further distinguish ANAs for the correct diagnosis. Some ANAs, like anti-Scl70, are highly specific but with a limited prevalence in the patients with SARD; on the other hand, certain ANAs like anti-DFS70 are negatively related with SARD.

Therefore, additional effort is required to identify the unknown ANAs and to study the corresponding clinical association.

Systemic scleroderma, or systemic sclerosis, is an autoimmune rheumatic disease characterized by excessive production and accumulation of collagen, called fibrosis, in the skin and internal organs and by injuries to small arteries. There are two major subgroups of systemic sclerosis based on the extent of skin involvement: limited and diffuse. The limited form affects areas below, but not above, the elbows and knees with or without involvement of the face. The diffuse form also affects the skin above the elbows and knees and can also spread to the torso. Visceral organs, including the kidneys, heart, lungs, and gastrointestinal tract can also be affected by the fibrotic process. Prognosis is determined by the form of the disease and the extent of visceral involvement. Patients with limited systemic sclerosis have a better prognosis than those with the diffuse form. Death is most often caused by lung, heart, and kidney involvement. There is also a slight increase in the risk of cancer.

About 60-95% of those with systemic scleroderma/systemic sclerosis have a positive ANA. In people who may have this condition, ANA subset tests can help to distinguish the two forms of the disease, the limited form versus the diffuse form.

Nuclear valosin-containing protein-like (synonyms: nuclear VCP-like, NVLp, Nuclear VCP-like protein, NVL) shows a high level of amino acid similarity to valosin-containing protein and locates exclusively in the nucleus. NVL belongs to the ATPase associated with various cellular activities protein family (AAA protein family). The major isoform of NVL contains two tandem AAA domains, a nuclear localization signal and a nucleolar localization signal (Fujiwara et al., “Structure and Function of the N-terminal Nucleolin Binding Domain of Nuclear Valosin-containing Protein-like 2 (NVL2) Harboring a Nucleolar Localization Signal,” JOURNAL OF BIOLOGICAL CHEMISTRY, pp. pp21732-21741, 17 Jun. 2011). It is ubiquitously expressed and mainly localized in the nucleolus and involved in biogenesis of the 60 S ribosomal subunit. It was reported that mutations in NVL may play a role in mental illness like major depressive disorder and schizophrenia (Wang et al., “The NVL gene confers risk for both major depressive disorder and schizophrenia in the Han Chinese population,” Progress in Neuro-Psychopharmacology and Biological Psychiatry, pp. 7-13, 1 Oct. 2015 and Ripke et al., “A mega-analysis of genome-wide association studies for major depressive disorder,” Molecular Psychiatry, pp. 497-511, April 2013.

CD2 antigen cytoplasmic tail-binding protein 2 (CD2BP2) is ubiquitously expressed in cells. Dependent on the cell type, it has important functions in the nucleus and the cytoplasm. In CD2 receptor expressing cells, like T cells, thymocytes and natural killer cells, the GYF-domain in CD2BP2 can interact with CD2 through the proline-rich motifs in cytoplasmic tail of CD2, which signals production of interleukin-2 (Nielsen et al., “Structural Basis for the Bifunctionality of the U5 snRNP 52K Protein (CD2BP2)”, Journal of Molecular Biology, pp. 369(4):902-908, 4 Apr. 2007 and Heinze at el., “Investigating the functional role of CD2BP2 in T cells”, International Immunology, vol. 19, no. 11, pp. 1313-1318, November 2007). Besides the function in immune response, CD2BP2 plays a role in pre-mRNA processing. CD2BP2 is also known as U5 snRNP 52K protein (U5-52K), because it is a component of U5 small nuclear ribonucleoprotein (snRNP), which together with other snRNPs forms the spliceosome (Nielsen et al., “Structural Basis for the Bifunctionality of the U5 snRNP 52K Protein (CD2BP2)”, Journal of Molecular Biology, pp. 369(4):902-908, 4 Apr. 2007). It was also reported that CD2BP2 is crucial for embryogenesis (Albert et al., “The GYF domain protein CD2BP2 is critical for embryogenesis and podocyte function,” Journal of Molecular Cell Biology, pp. 7(5), 402-414, 16 Jun. 2015). It has not been reported in the prior art that CD2BP2/U5-52K is associated with autoimmune diseases.

Zonula occludens-1 (ZO-1), also known as Tight junction protein-1 (TJP1) is a 220-kD peripheral membrane protein that is encoded by the TJP1 gene in humans. It belongs to the family of zona occludens proteins (ZO-1, ZO-2, and ZO-3), which are tight junction-associated proteins and of which ZO-1 is the first to be cloned. It was first isolated in 1986 by Stevenson and Goodenough using a monoclonal antibody raised in rodent liver to recognize a 225-kD polypeptide in whole liver homogenates and in tight junction-enriched membrane fractions. TJP1 is specifically enriched at the tight junctions of epithelial and endothelial cells. It belongs to the membrane associated guanylate kinase (MAGUK) protein family and shuffles between the nucleus and tight junctions (Hou, “Paracellular Channel Formation,” in The Paracellular Channel, Academic Press, 2019, pp. 9-27).

### SUMMARY OF THE INVENTION

The problem underlying the present invention is to provide methods, related assays and related reagents of unknown ANAs and/or to provide or define new ANAs for diagnosis and corresponding diagnostic methods, assays and related reagents that can be used for diagnosis, especially for the diagnosis of systemic autoimmune rheumatic diseases.

Another problem underlying the present invention is to increase the sensitivity of diagnostics for the diagnosis of systemic autoimmune rheumatic diseases, preferably for distinguishing autoimmune types of systemic autoimmune rheumatic diseases.

In a 1^{st} aspect, the problem underlying the present invention is solved by a method for detecting in a sample, preferably from a patient, an autoantibody binding to a polypeptide having the sequence SEQ ID NO: 1, SEQ ID NO: 11 or SEQ ID NO: 24 or a variant thereof.

The invention also includes the following embodiments:

1. A method for detecting in a sample, preferably from a patient, an autoantibody binding to a polypeptide having the sequence SEQ ID NO: 1, SEQ ID NO: 11 or SEQ ID NO: 24 or a variant thereof.

2. A polypeptide having the sequence SEQ ID NO: 1, SEQ ID NO: 11 or SEQ ID NO: 24 or a variant thereof, wherein the polypeptide is preferably immobilized, more preferably on a solid carrier, and/or wherein the polypeptide is a recombinant protein, preferably a purified recombinant protein.

3. A carrier, preferably a solid carrier, comprising the polypeptide according to embodiment 2, wherein preferably the polypeptide is immobilized on the carrier, and/or wherein the polypeptide is preferably a recombinant protein, more preferably a purified recombinant protein.

4. An autoantibody, preferably an isolated autoantibody, binding to a polypeptide having the sequence SEQ ID NO: 1, SEQ ID NO: 11 or SEQ ID NO: 24 or a variant thereof, wherein the autoantibody, preferably the isolated autoantibody, is preferably in complex with the polypeptide according to embodiment 2.

5. Autoantibody according to embodiment 4 wherein the autoantibody, preferably the isolated autoantibody, is diluted in a buffer solution or is included in a diluted sample, preferably patient sample and/or wherein the autoantibody, preferably the isolated autoantibody, is in solution with one or more, preferably all reagents from the group comprising stabilizers, preferably a set of stabilizers, washing buffer, antidegradants.

6. A use of the autoantibody according to embodiment 4 or 5 for the diagnosis of a disease preferably of a systemic autoimmune rheumatic disease, more preferably of systemic sclerosis.

7. A method for isolating an autoantibody binding to a polypeptide having the sequence SEQ ID NO: 1, SEQ ID NO: 11 or SEQ ID NO: 24 or a variant thereof, comprising the steps

a) contacting a sample comprising the autoantibody with a polypeptide according to embodiment 2 under conditions compatible with formation of a complex, wherein said autoantibody binds to said polypeptide,

b) isolating the complex formed in step a),

c) dissociating the complex isolated in step b) and

d) separating the autoantibody from the polypeptide.

8. A kit comprising the polypeptide according to embodiment 2, wherein preferably the kit comprises in addition a means for detecting a complex comprising the polypeptide according to embodiment 2 and an antibody binding to said polypeptide.

9. A kit comprising

a) the carrier according to embodiment 3 and a means for detecting an autoantibody bound to the carrier, which means is preferably a secondary antibody, or a polypeptide having the sequence SEQ ID NO: 1, SEQ ID NO: 11 or SEQ ID NO: 24 or a variant thereof,

or

b) a diagnostically useful carrier comprising a means for capturing an autoantibody in a liquid solution, preferably a non-labeled secondary antibody, and a means for detecting an autoantibody bound to the carrier, preferably a polypeptide having the sequence SEQ ID NO: 1, SEQ ID NO: 11 or SEQ ID NO: 24 or a variant thereof, more preferably with a detectable label,

wherein the kit preferably comprises in addition to a) or b) one or more, preferably all reagents from the group comprising calibrators, preferably a set of calibrators, washing buffer, a positive control and a negative control.

10. A use of a polypeptide according to embodiment 2 or a carrier according to embodiment 3 or an autoantibody according to embodiment 4 or 5 or an antibody to a polypeptide having the sequence SEQ ID NO: 1, SEQ ID NO: 11 or SEQ ID NO: 24 or a variant thereof, for the manufacture of a kit, medical device, preferably diagnostic device, preferably for the diagnosis of a disease, more preferably for the diagnosis of a systemic autoimmune rheumatic disease, even more preferably for the diagnosis of systemic sclerosis.

11. The method or autoantibody according to any of embodiments 1, 5 and 7, wherein the sample is a bodily fluid comprising antibodies, preferably selected from the group comprising whole blood, plasma, serum, cerebrospinal fluid and saliva.

12. The method, use or kit according to any of embodiments 1 and 6 to 11 wherein the autoantibody or complex is detected using a method selected from the group comprising immunodiffusion techniques, immunoelectrophoretic techniques, light scattering immunoassays, agglutination techniques, labeled immunoassays such as those from the group comprising radiolabeled immunoassay, enzyme immunoassays, more preferably ELISA, chemiluminescence immunoassays, preferably electrochemiluminescence immunoassay, and immunofluorescence, preferably indirect immunofluorescence.

13. A method comprising the step contacting a medical or diagnostic device comprising a polypeptide having the sequence SEQ ID NO: 1, SEQ ID NO: 11 or SEQ ID NO: 24 or a variant thereof with a buffered solution comprising an antibody binding specifically to said polypeptide having the sequence SEQ ID NO: 1, SEQ ID NO: 11 or SEQ ID NO: 24 or a variant thereof, which solution is not a sample from a patient in need of a diagnosis comprising an unknown concentration of said antibody, wherein preferably

a) the concentration of the antibody in the solution is known, and/or

b) the antibody is a recombinant antibody and/or

c) the medical or diagnostic device is contacted with two or more solutions comprising the antibody, wherein the two or more solutions have a different concentration of the antibody, and/or

d) the antibody is recognized by secondary antibodies binding specifically to IgG class antibodies,

followed by detection of a signal which indicates whether or not the antibody has bound to the polypeptide, optionally a signal relating to the concentration of the antibody in the solution or solutions.

14. Use of a sample, preferably a patient sample, which is preferably diluted, comprising an autoantibody according to embodiment 4 or 5 as a positive control, preferably for the diagnosis of a disease, more preferably for the diagnosis of a systemic autoimmune rheumatic disease, even more preferably for the diagnosis of systemic sclerosis.

15. Diluted sample comprising an autoantibody according to embodiment 4, 5, 10 or 11 wherein the sample is preferably diluted in an aqueous buffer solution and/or wherein the diluted sample comprises in addition one or more, preferably all reagents from the group comprising calibrators, preferably a set of calibrators, a stabilizer, washing buffer, a positive control and a negative control.

### BRIEF DESCRIPTION OF THE DRAWINGS

FIG. 1 shows IFA with HEp-2 cells or monkey liver with 1:100 diluted patient sera (PS).

FIG. 2 shows immunoprecipitation of patient (PS1-3) or control sera (CS) with HEp2 extract.

FIG. 3A shows immunoblots with anti-His-tag (1:2000), patient sera (1:200) and healthy control sera (1:200) with recombinant antigens purified NVL-His expressed in E. coli and PS1.

FIG. 3B shows immunoblots with anti-His-tag (1:2000), patient sera (1:200) and healthy control sera (1:200) with recombinant antigens purified His-CD2BP2 expressed in E. coli and PS2.

FIG. 3C shows immunoblots with anti-His-tag (1:2000), patient sera (1:200) and healthy control sera (1:200) with recombinant antigens purified His-TJP1 aa1-575 expressed in E. coli and PS3.

FIG. 3D shows immunoblots with anti-His-tag (1:2000), patient sera (1:200) and healthy control sera (1:200) with recombinant antigens full-length TJP1-His (HEK293 cell lysate) or His-TJP1 aa1-575 (E. coli lysate).

FIG. 4 shows IFA with NVL-His, TJP1-His or control transfected HEK293 cells and PS1 (1:100) or PS3, respectively (1:10).

FIG. 5 shows IFA neutralization assay with patient sera and HEp-2 cells.

FIG. 6A shows colocalization analysis with anti-TJP1 antibody (mouse, monoclonal) and PS3 in HEp-2 cells.

FIG. 6B shows colocalization analysis with anti-TJP1 antibody (mouse, monoclonal) and PS3 in transfected HEK293 cells overexpressing TJP1-His.

FIG. 6C shows colocalization analysis with anti-TJP1 antibody (mouse, monoclonal) and PS3 in control HEK293 cells transfected with empty vector, at magnification 200×.

FIG. 6D shows colocalization analysis with anti-TJP1 antibody (mouse, monoclonal) and PS3 in control HEK293 cells transfected with empty vector, at magnification 3200×.

FIG. 7A shows immunoblots with E. coli lysates containing NVL fragments (aa1-117, aa106-292, aa281-560, aa549-856).

FIG. 7B shows immunoblots with E. coli lysates containing CD2BP2 fragments (aa117-260, aa249-341).

FIG. 7C shows immunoblots with E. coli lysates containing TJP1 fragments (aa1-156, aa145-306, aa295-575).

FIG. 8A shows immunoblots with purified NVL-His.

FIG. 8B shows immunoblots with His-CD2BP2.

FIG. 8C shows immunoblots with His-TJP1 aa1-575.

### DETAILED DESCRIPTION OF THE INVENTION

In a preferred embodiment, the polypeptide consists of or comprises a sequence according to SEQ ID NO: 1 or a variant thereof. In an also preferred embodiment, the polypeptide consists of or comprises at least one of the sequences according to SEQ ID NO: 1, SEQ ID NO: 35, SEQ ID NO: 38, SEQ ID NO: 41, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 86, SEQ ID NO: 87, SEQ ID NO: 88, SEQ ID NO: 89, SEQ ID NO: 90, SEQ ID NO: 91, SEQ ID NO: 92 and SEQ ID NO: 93. In a more preferred embodiment, the polypeptide consists of or comprises at least one of the sequences according to SEQ ID NO: 86, SEQ ID NO: 87, SEQ ID NO: 88, SEQ ID NO: 89, SEQ ID NO: 90, SEQ ID NO: 91, SEQ ID NO: 92 and SEQ ID NO: 93. In a further preferred embodiment, the polypeptide consists of or comprises two, three, four, five, six, seven or all of the sequences according to SEQ ID NO: 86, SEQ ID NO: 87, SEQ ID NO: 88, SEQ ID NO: 89, SEQ ID NO: 90, SEQ ID NO: 91, SEQ ID NO: 92 and SEQ ID NO: 93.

In a preferred embodiment, the autoantibody is an IgG class antibody.

In a preferred embodiment, the sample is a bodily fluid comprising antibodies, preferably selected from the group comprising whole blood, plasma, serum, cerebrospinal fluid and saliva. Preferably, the sample is from a mammalian, more preferably human patient.

In a preferred embodiment, the patient has one or more, preferably two or more symptoms selected from the group comprising muscle aches, fatigue, joint stiffness, swelling and redness over the joints, mild fever, trouble concentrating, rashes, including a “butterfly” rash across the cheeks, hair loss, numbness and tingling in the hands and feet, development of nodules under the skin, malaise, sun sensitivity.

In a preferred embodiment, the patient has or is supposed to have a systemic autoimmune rheumatic disease, preferably SSc.

In a preferred embodiment, the antibody, preferably autoantibody, is detected using a method from the group comprising immunodiffusion, immunoelectrophoresis, light scattering immunoassays, agglutination, labeled immunoassays such as those from the group comprising radiolabeled immunoassays, enzyme immunoassays such as colorimetric assays, chemiluminescence immunoassays and immunofluorescence, preferably immunofluorescence, more preferably indirect immunofluorescence. In a preferred embodiment, the antibody, preferably autoantibody, is detected using a recombinant indirect immunofluorescence technique with biochips.

In a 2^{nd} aspect, the problem underlying the present invention is solved by a polypeptide having the sequence SEQ ID NO: 1, SEQ ID NO: 11 or SEQ ID NO: 24 or a variant thereof, wherein the polypeptide is preferably immobilized, more preferably on a solid carrier, and/or wherein the polypeptide is a recombinant protein, preferably a purified recombinant protein.

In a preferred embodiment, the polypeptide according to the present invention consists of or comprises a sequence according to SEQ ID NO: 1 or a variant thereof. In an also preferred embodiment, the polypeptide consists of or comprises at least one of the sequences according to SEQ ID NO: 1, SEQ ID NO: 35, SEQ ID NO: 38, SEQ ID NO: 41, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 86, SEQ ID NO: 87, SEQ ID NO: 88, SEQ ID NO: 89, SEQ ID NO: 90, SEQ ID NO: 91, SEQ ID NO: 92 and SEQ ID NO: 93. In a more preferred embodiment, the polypeptide consists of or comprises at least one of the sequences according to SEQ ID NO: 86, SEQ ID NO: 87, SEQ ID NO: 88, SEQ ID NO: 89, SEQ ID NO: 90, SEQ ID NO: 91, SEQ ID NO: 92 and SEQ ID NO: 93. In a further preferred embodiment, the polypeptide consists of or comprises two, three, four, five, six, seven or all of the sequences according to SEQ ID NO: 86, SEQ ID NO: 87, SEQ ID NO: 88, SEQ ID NO: 89, SEQ ID NO: 90, SEQ ID NO: 91, SEQ ID NO: 92 and SEQ ID NO: 93.

In a preferred embodiment, the polypeptide according to the present invention is isolated, purified and/or immobilized.

In a preferred embodiment, the polypeptide according to the present invention is a recombinant protein, preferably a purified recombinant protein.

In a preferred embodiment, the polypeptide according to the present invention is for use in a treatment of a disease, wherein the disease is preferably SARD, more preferably SSc.

In a preferred embodiment, the polypeptide according to the present invention is used in a treatment of a disease, wherein the disease is preferably SARD, more preferably SSc.

In a 3^{rd} aspect, the problem underlying the present invention is solved by a carrier, preferably a solid carrier, comprising a polypeptide having the sequence SEQ ID NO: 1, SEQ ID NO: 11 or SEQ ID NO: 24 or a variant thereof, wherein preferably the polypeptide is immobilized on the carrier, and/or wherein the polypeptide is preferably a recombinant protein, more preferably a purified recombinant protein.

In a preferred embodiment, the carrier comprises a polypeptide consisting of or comprising at least one of the sequences according to SEQ ID NO: 1, SEQ ID NO: 35, SEQ ID NO: 38, SEQ ID NO: 41, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 86, SEQ ID NO: 87, SEQ ID NO: 88, SEQ ID NO: 89, SEQ ID NO: 90, SEQ ID NO: 91, SEQ ID NO: 92 and SEQ ID NO: 93. In a more preferred embodiment, the carrier comprises a polypeptide that consists of or comprises at least one of the sequences according to SEQ ID NO: 86, SEQ ID NO: 87, SEQ ID NO: 88, SEQ ID NO: 89, SEQ ID NO: 90, SEQ ID NO: 91, SEQ ID NO: 92 and SEQ ID NO: 93. In a further preferred embodiment, the carrier comprises a polypeptide that consists of or comprises two, three, four, five, six, seven or all of the sequences according to SEQ ID NO: 86, SEQ ID NO: 87, SEQ ID NO: 88, SEQ ID NO: 89, SEQ ID NO: 90, SEQ ID NO: 91, SEQ ID NO: 92 and SEQ ID NO: 93.

In a preferred embodiment, the carrier according to the present invention is a diagnostically useful carrier.

In a preferred embodiment, the carrier according to the present invention is a solid carrier, wherein the polypeptide is immobilized on the carrier, and wherein the polypeptide is preferably a recombinant protein, more preferably a purified recombinant protein.

In a preferred embodiment, the carrier according to the present invention is selected from the group comprising a bead, preferably a paramagnetic bead, a test strip, a microtiter plate, a membrane, preferably from the group comprising western blot, line blot and dot blot, a lateral flow device, a glass surface, a slide for microscopy, a microarray and a biochip and is preferably a slide for microscopy. In a further preferred embodiment, the carrier is a line blot or a biochip.

In a preferred embodiment, any immobilized polypeptide is expressed by a cell immobilized on the carrier according to the present invention, preferably a fixed cell, or is a recombinant or isolated polypeptide immobilized on the carrier according to the present invention.

In a preferred embodiment, any immobilized polypeptide is expressed by a cell immobilized on the carrier according to the present invention and the carrier further comprises a mock-transfected cell.

In a preferred embodiment, an autoantibody binding specifically to a polypeptide having the sequence SEQ ID NO: 1, SEQ ID NO: 11 or SEQ ID NO: 24 or a variant thereof, is bound to the polypeptide having the sequence SEQ ID NO: 1, SEQ ID NO: 11 or SEQ ID NO: 24 or a variant thereof and optionally a secondary antibody comprising a label.

In a preferred embodiment, an autoantibody binding specifically to a polypeptide having the sequence SEQ ID NO: 1 or a variant thereof, is bound to a polypeptide consisting of or comprising at least one of the sequences according to SEQ ID NO: 1, SEQ ID NO: 35, SEQ ID NO: 38, SEQ ID NO: 41, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 86. SEQ ID NO: 87, SEQ ID NO: 88, SEQ ID NO: 89. SEQ ID NO: 90, SEQ ID NO: 91, SEQ ID NO: 92 and SEQ ID NO: 93 and optionally a secondary antibody comprising a label.

In a preferred embodiment, the carrier according to the present invention is a test strip comprising spatially separate reagents which includes a polypeptide having the sequence SEQ ID NO: 1, SEQ ID NO: 11 or SEQ ID NO: 24 or a variant thereof, and one or more from the group comprising one or more calibrators and a reagent indicating the presence of a secondary antibody, preferably a secondary antibody recognizing IgG class antibodies.

In a 4^{th} aspect, the problem underlying the present invention is solved by an autoantibody, preferably an isolated autoantibody, binding to a polypeptide having the sequence SEQ ID NO: 1, SEQ ID NO: 11 or SEQ ID NO: 24 or a variant thereof, wherein the autoantibody, preferably the isolated autoantibody, is preferably in complex with a polypeptide having the sequence SEQ ID NO: 1. SEQ ID NO: 11 or SEQ ID NO: 24 or a variant thereof.

In a preferred embodiment the autoantibody is isolated.

In a preferred embodiment, the autoantibody, preferably the isolated autoantibody, is diluted in a buffer solution or is included in a diluted sample, preferably patient sample and/or the autoantibody, preferably the isolated autoantibody, is in solution with one or more, preferably all reagents from the group comprising stabilizers, preferably a set of stabilizers, washing buffer, antidegradants.

In a preferred embodiment, the autoantibody or complex is detected using a method selected from the group comprising immunodiffusion techniques, immunoelectrophoretic techniques, light scattering immunoassays, agglutination techniques, labeled immunoassays such as those from the group comprising radiolabeled immunoassay, enzyme immunoassays, more preferably ELISA, chemiluminescence immunoassays, preferably electrochemiluminescence immunoassay, and immunofluorescence, preferably indirect immunofluorescence.

In a 5^{th} aspect, the problem underlying the present invention is solved by a use of an autoantibody according to the present invention for the diagnosis of a disease, preferably of a systemic autoimmune rheumatic disease, more preferably of systemic sclerosis.

In a preferred embodiment, the disease is a systemic autoimmune rheumatic disease or a connective tissue disease, more preferably selected from the group comprising systemic sclerosis, anti-synthetase syndrome (ASS), rheumatoid arthritis (RA), systemic lupus erythematosus, primary Sjögren syndrome (pSS), inclusion body myositis (IBM), myositis, mixed connective tissue disease (MCTD) or undifferentiated connective tissue disease (UCTD).

In a preferred embodiment, the disease is associated with one or more, preferably two or more symptoms selected from the group comprising skin thickening and tightness, puffy finger, sclerodactyly, fingertip lesions, hyper—or hypopigmentation of the skin, swelling, general pain, chest pain, calcinosis, Raynaud's phenomenon, telangiectasias, abnormal nailfold capillaries, shortness of breath, pulmonary arterial hypertension, pulmonary fibrosis, muscle aches, fatigue, joint stiffness, swelling and redness over the joints, mild fever, trouble concentrating, rashes, including a “butterfly” rash across the cheeks, hair loss, numbness and tingling in the hands and feet, development of nodules under the skin, malaise, sun sensitivity.

In a preferred embodiment, the use comprises the step detecting in a sample an autoantibody binding to a polypeptide having the sequence SEQ ID NO: 1, SEQ ID NO: 11 or SEQ ID NO: 24 or a variant thereof. In a further preferred embodiment, the use comprises the step detecting in a sample an autoantibody binding to a polypeptide consisting of or comprising at least one of the sequences according to SEQ ID NO: 1, SEQ ID NO: 35, SEQ ID NO: 38, SEQ ID NO: 41, SEQ ID NO: 44. SEQ ID NO: 45, SEQ ID NO: 46, SEQ ID NO: 47. SEQ ID NO: 48, SEQ ID NO: 49, SEQ ID NO: 50, SEQ ID NO: 51, SEQ ID NO: 52, SEQ ID NO: 86, SEQ ID NO: 87, SEQ ID NO: 88, SEQ ID NO: 89, SEQ ID NO: 90, SEQ ID NO: 91, SEQ ID NO: 92 and SEQ ID NO: 93.

In a 6^{th} aspect, the problem underlying the present invention is solved by a method for isolating an autoantibody binding to a polypeptide having the sequence SEQ ID NO: 1, SEQ ID NO: 11 or SEQ ID NO: 24 or a variant thereof, comprising the steps
a) contacting a sample comprising the autoantibody with a polypeptide having the sequence SEQ ID NO: 1, SEQ ID NO: 11 or SEQ ID NO: 24 or a variant thereof, under conditions compatible with formation of a complex, wherein said autoantibody binds to said polypeptide,
b) isolating the complex formed in step a),
c) dissociating the complex isolated in step b) and
d) separating the autoantibody from the polypeptide.

In a 7^{th} aspect, the problem underlying the present invention is solved by a kit comprising a polypeptide having the sequence SEQ ID NO: 1, SEQ ID NO: 11 or SEQ ID NO: 24 or a variant thereof.

In a preferred embodiment, the kit according to the present invention comprises a polypeptide consisting of or comprising at least one of the sequences according to SEQ ID NO: 1. SEQ ID NO: 35, SEQ ID NO: 38, SEQ ID NO: 41, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52. SEQ ID NO: 86, SEQ ID NO: 87, SEQ ID NO: 88. SEQ ID NO: 89, SEQ ID NO: 90, SEQ ID NO: 91, SEQ ID NO: 92 and SEQ ID NO: 93.

In a preferred embodiment, the kit comprises in addition a means for detecting a complex comprising the polypeptide having the sequence SEQ ID NO: 1, SEQ ID NO: 11 or SEQ ID NO: 24 or a variant thereof, and an antibody binding to said polypeptide.

In an 8^{th} aspect, the problem underlying the present invention is solved by a kit comprising
a) the carrier according to the present invention and a means for detecting an autoantibody bound to the carrier, which means is preferably a secondary antibody, or a polypeptide having the sequence SEQ ID NO: 1, SEQ ID NO: 11 or SEQ ID NO: 24 or a variant thereof, preferably a polypeptide consisting of or comprising at least one of the sequences according to SEQ ID NO: 1, SEQ ID NO: 35, SEQ ID NO: 38, SEQ ID NO: 41. SEQ ID NO: 44, SEQ ID NO: 45, SEQ ID NO: 46. SEQ ID NO: 47, SEQ ID NO: 48, SEQ ID NO: 49, SEQ ID NO: 50, SEQ ID NO: 51, SEQ ID NO: 52, SEQ ID NO: 86, SEQ ID NO: 87, SEQ ID NO: 88, SEQ ID NO: 89, SEQ ID NO: 90, SEQ ID NO: 91. SEQ ID NO: 92 and SEQ ID NO: 93,

or
b) a diagnostically useful carrier comprising a means for capturing an autoantibody according to the present invention in a liquid solution, preferably a non-labeled secondary antibody, and a means for detecting an autoantibody bound to the carrier, preferably a polypeptide having the sequence SEQ ID NO: 1, SEQ ID NO: 11 or SEQ ID NO: 24 or a variant thereof, more preferably a polypeptide consisting of or comprising at least one of the sequences according to SEQ ID NO: 1, SEQ ID NO: 35, SEQ ID NO: 38. SEQ ID NO: 41, SEQ ID NO: 44, SEQ ID NO: 45. SEQ ID NO: 46, SEQ ID NO: 47. SEQ ID NO: 48, SEQ ID NO: 49, SEQ ID NO: 50. SEQ ID NO: 51, SEQ ID NO: 52, SEQ ID NO: 86, SEQ ID NO: 87, SEQ ID NO: 88, SEQ ID NO: 89, SEQ ID NO: 90, SEQ ID NO: 91, SEQ ID NO: 92 and SEQ ID NO: 93, even more preferably with a detectable label,

wherein the kit preferably comprises in addition to a) or b) one or more, preferably all reagents from the group comprising calibrators, preferably a set of calibrators, washing buffer, a positive control and a negative control.

In a preferred embodiment, the kit according to the present invention is a kit for the diagnosis of a disease, preferably SARD, more preferably SSc, which kit comprises a polypeptide according to the present invention, wherein the kit preferably comprises in addition a means for detecting a complex comprising the polypeptide according to the present invention and an antibody binding to said polypeptide according to the present invention.

In a preferred embodiment, the kit according to the present invention comprises in addition a means for detecting an autoantibody to another SARD-related autoantigen, preferably selected from the group comprising dsDNA, nucleosomes, histones, SS-A, Ro-52, SSB, RNP/Sm, Sm, Mi-2α, Mi-2β, Ku, CENP A, CENP B, Sp100, PML, Scl-70, PM100, PM75, RP11, RP155, gp210, PCNA, Fibrillarin, NOR90, Th/To, PDGFR and DSF70. Preferably, the kit can comprise one, two or more additional means for detecting an autoantibody to one, two or more further SARD-related autoantigens, respectively, wherein the one, two or more further SARD-related autoantigens are preferably selected from the group comprising dsDNA, nucleosomes, histones, SS-A, Ro-52, SSB, RNP/Sm, Sm, Mi-2α, Mi-2β, Ku, CENP A, CENP B, Sp100, PML, Scl-70, PM100, PM75, RP11, RP155, gp210, PCNA, Fibrillarin, NOR90, Th/To, PDGFR and DSF70.

In a 9^{th} aspect, the problem underlying the present invention is solved by a use of a polypeptide, a carrier or an autoantibody according to the present invention or an antibody to a polypeptide having the sequence SEQ ID NO: 1, SEQ ID NO: 11 or SEQ ID NO: 24 or a variant thereof, for the manufacture of a kit, medical device, preferably diagnostic device, preferably for the diagnosis of a disease, more preferably for the diagnosis of SARD, even more preferably for the diagnosis of SSc.

In a 10^{th} aspect, the problem underlying the present invention is solved by a pharmaceutical composition or medical device, preferably diagnostic device, comprising a polypeptide having the sequence SEQ ID NO: 1, SEQ ID NO: 11 or SEQ ID NO: 24 or a variant thereof.

In a preferred embodiment, the pharmaceutical composition or medical device comprises a polypeptide that consists of or comprises a sequence according to SEQ ID NO: 1 or a variant thereof. In an also preferred embodiment, this polypeptide consists of or comprises at least one of the sequences according to SEQ ID NO: 1, SEQ ID NO: 35, SEQ ID NO: 38, SEQ ID NO: 41, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 86, SEQ ID NO: 87. SEQ ID NO: 88. SEQ ID NO: 89, SEQ ID NO: 90, SEQ ID NO: 91, SEQ ID NO: 92 and SEQ ID NO: 93. In a more preferred embodiment, this polypeptide consists of or comprises at least one of the sequences according to SEQ ID NO: 86, SEQ ID NO: 87, SEQ ID NO: 88, SEQ ID NO: 89, SEQ ID NO: 90, SEQ ID NO: 91, SEQ ID NO: 92 and SEQ ID NO: 93. In a further preferred embodiment, this polypeptide consists of or comprises two, three, four, five, six, seven or all of the sequences according to SEQ ID NO: 86, SEQ ID NO: 87, SEQ ID NO: 88, SEQ ID NO: 89, SEQ ID NO: 90, SEQ ID NO: 91, SEQ ID NO: 92 and SEQ ID NO: 93.

In a preferred embodiment, the pharmaceutical composition or the medical device further comprises one or more adjuvants.

In a preferred embodiment, the medical device is selected from the group comprising a glass slide, preferably for microscopy, a biochip, a microtiter plate, a lateral flow device, a test strip, a membrane, preferably a line blot, a chromatography column and a bead, preferably a magnetic or fluorescent bead.

In an 11^{th} aspect, the problem underlying the present invention is solved by a method comprising the step contacting a medical or diagnostic device comprising a polypeptide having the sequence SEQ ID NO: 1, SEQ ID NO: 11 or SEQ ID NO: 24 or a variant thereof with a buffered solution comprising an antibody binding specifically to said polypeptide having the sequence SEQ ID NO: 1, SEQ ID NO: 11 or SEQ ID NO: 24 or a variant thereof, which solution is not a sample from a patient in need of a diagnosis comprising an unknown concentration of said antibody, wherein preferably
a) the concentration of the antibody in the solution is known, and/or
b) the antibody is a recombinant antibody and/or
c) the medical or diagnostic device is contacted with two or more solutions comprising the antibody, wherein the two or more solutions have a different concentration of the antibody, and/or
d) the antibody is recognized by secondary antibodies binding specifically to IgG class antibodies,

followed by detection of a signal which indicates whether or not the antibody has bound to the polypeptide, optionally a signal relating to the concentration of the antibody in the solution or solutions.

In a 12^{th} aspect, the problem underlying the present invention is solved by a use of a sample, preferably a patient sample, which is preferably diluted, comprising an autoantibody according to the present invention as a positive control.

In a 13^{th} aspect, the problem underlying the present invention is solved by a diluted sample comprising an autoantibody according to the present invention, wherein the sample is preferably diluted in an aqueous buffer solution and/or wherein the diluted sample comprises in addition one or more, preferably all reagents from the group comprising calibrators, preferably a set of calibrators, a stabilizer, washing buffer, a positive control and a negative control.

In a 14^{th} aspect, the problem underlying the present invention is solved by an aqueous solution comprising an autoantibody according to the present invention.

In a preferred embodiment, the aqueous solution comprises a sample from a human patient.

In a preferred embodiment, the solution comprises a non-physiological buffer or a physiological buffer at concentrations above physiological levels and more preferably has a pH between 5 and 9, preferably 6 to 8.

In a 15^{th} aspect, the problem underlying the present invention is solved by a device for removing an autoantibody according to the present invention, from blood, preferably serum of a patient, wherein the device comprises a carrier according to the present invention.

In a 16^{th} aspect, the problem underlying the present invention is solved by an ex vivo method for removing an autoantibody according to the present invention, from blood, preferably serum of a patient.

According to the present invention one or more epitopes recognized by anti-NVL autoantibodies is or are located on one, two, three, four, five, six, seven or all C-terminal fragments according to SEQ ID NO: 86. SEQ ID NO: 87, SEQ ID NO: 88, SEQ ID NO: 89, SEQ ID NO: 90, SEQ ID NO: 91, SEQ ID NO: 92 and SEQ ID NO: 93.

The present invention is based on the surprising finding of the inventors that autoantibodies to NVL, CD2BP2 and TJP1 are present and detectable in samples. They can be detected using immunoassays and can be used to set up serological immunoassays for the diagnosis of diseases, especially SARD, specifically SSc. A carrier comprising immobilized polypeptides of NVL, CD2BP2 or TJP1 or variants thereof may be used for detecting such autoantibodies.

According to the present invention, the term “a polypeptide having the sequence SEQ ID NO: 1, SEQ ID NO: 11 or SEQ ID NO: 24 or a variant thereof” as used herein, refers to a polypeptide having the sequence SEQ ID NO: 1 or a variant thereof, a polypeptide having the sequence SEQ ID NO: 11 or a variant thereof or a polypeptide having the sequence SEQ ID NO: 24 or a variant thereof.

According to the present invention, the term “a polypeptide having the sequence” as used herein, refers to a polypeptide consisting of or comprising said sequence.

According to the present invention, the term “a polypeptide having the sequence SEQ ID NO: 1 or a variant thereof” as used herein, refers to a polypeptide consisting of or comprising at least one of the sequences according to SEQ ID NO: 1, SEQ ID NO: 35, SEQ ID NO: 38, SEQ ID NO: 41, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 86, SEQ ID NO: 87, SEQ ID NO: 88, SEQ ID NO: 89, SEQ ID NO: 90, SEQ ID NO: 91, SEQ ID NO: 92 and SEQ ID NO: 93.

According to the present invention, a polypeptide having the sequence SEQ ID NO: 1, SEQ ID NO: 11 or SEQ ID NO: 24 or a variant thereof is used to detect an autoantibody to SEQ ID NO: 1, SEQ ID NO: 11 or SEQ ID NO: 24 or a variant thereof, for example by coating the diagnostically useful carrier according to the present invention with a polypeptide having the sequence SEQ ID NO: 1, SEQ ID NO: 11 or SEQ ID NO: 24 or a variant thereof or by using a soluble polypeptide having the sequence SEQ ID NO: 1, SEQ ID NO: 11 or SEQ ID NO: 24 or a variant thereof, which may be detected if bound to the autoantibody. In a preferred embodiment, any sequence referred to is understood to be presented as part of a polypeptide or a similar form which may be used to detect the autoantibody of interest.

Preferably a polypeptide having the sequence SEQ ID NO: 1, SEQ ID NO: 11 or SEQ ID NO: 24 or a variant thereof is immobilized on a solid phase of the carrier. It may be directly immobilized on the solid phase when contacted with the sample, but a competitive assay, a capture bridge assay, an immunometric assay, a class-specific second antibody on the solid phase, a class capture assay, direct or indirect may also be used. The principle of each of these formats is detailed in The Immunoassay Handbook, 3^{rd} edition, edited by David Wild, Elsevier, 2005. More preferably, the solid phase is a test strip or a well of a microtiter plate for ELISA, preferably a well of a microtiter plate for ELISA.

The teachings of the present invention may not only be carried out using the polypeptides, in particular a polypeptide comprising the native sequence of a polypeptide referred to such as human NVL-isoform1 (SEQ ID NO: 1), human CD2BP2 (SEQ ID NO: 11) or human TJP1-isoform2 (SEQ ID NO: 24) or polypeptides having the exact sequences referred to in this application explicitly, for example by function, name, sequence or accession number, or implicitly, but also using variants of such polypeptides.

In a preferred embodiment, the teachings of the present invention are carried out using a polypeptide having the sequence SEQ ID NO: 1 or a variant thereof, having the sequence SEQ ID NO: 11 or a variant thereof or having the sequence SEQ ID NO: 24 or a variant thereof.

In a preferred embodiment, the teachings of the present invention are carried out using a polypeptide having the sequence SEQ ID NO: 10, i.e. a polypeptide referred to such as NVL[human]-IF1 fused to a hexa Histidin-tag (H6), or a variant thereof.

In a preferred embodiment, the teachings of the present invention are carried out using a polypeptide having the sequence of a polypeptide referred to such as H8-NVL[human](aa1-117) (SEQ ID NO: 49), H8-NVL[human](aa106-292) (SEQ ID NO: 50), H8-NVL[human](aa281-560) (SEQ ID NO: 51), H8-NVL[human](aa549-856) (SEQ ID NO: 52), or a variant thereof.

In a preferred embodiment, the teachings of the present invention are carried out using a polypeptide consisting of or comprising at least one of the sequences according to SEQ ID NO: 1, SEQ ID NO: 35, SEQ ID NO: 38, SEQ ID NO: 41, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 86, SEQ ID NO: 87, SEQ ID NO: 88, SEQ ID NO: 89, SEQ ID NO: 90, SEQ ID NO: 91, SEQ ID NO: 92 and SEQ ID NO: 93.

In a preferred embodiment, the teachings of the present invention are carried out using a polypeptide consisting of or comprising at least one of the sequences according to SEQ ID NO: 86, SEQ ID NO: 87, SEQ ID NO: 88, SEQ ID NO: 89, SEQ ID NO: 90, SEQ ID NO: 91, SEQ ID NO: 92 and SEQ ID NO: 93.

In a preferred embodiment, the teachings of the present invention are carried out using a polypeptide having the sequence of a polypeptide referred to such as CD2BP2[human] fused to a N-terminal octa Histidin-tag (H8) (SEQ ID NO: 23) or a variant thereof.

In a preferred embodiment, the teachings of the present invention are carried out using a polypeptide having the sequence of a polypeptide referred to such as H8-CD2BP2[human](aa117-260) (SEQ ID NO: 66), H8-CD2BP2[human](aa249-341) (SEQ ID NO: 67) or a variant thereof.

In a preferred embodiment, the teachings of the present invention are carried out using a polypeptide having the sequence of a polypeptide referred to such as pET24d-N-TJP1[human](aa1-575) fused to a N-terminal octa Histidin-tag (H8) (SEQ ID NO: 82) or a variant thereof.

In a preferred embodiment, the teachings of the present invention are carried out using a polypeptide having the sequence of a polypeptide referred to such as TJP1[human]-isoform2 fused to a C-terminal octa Histidin-tag (H8) (SEQ ID NO: 32), H8-TJP1[human](aa1-156) (SEQ ID NO: 83), H8-TJP1[human](aa144-306) (SEQ ID NO: 84), H8-TJP1[human](aa294-575) (SEQ ID NO: 85) or a variant thereof.

In a preferred embodiment, the term “variant”, as used herein, may refer to at least one fragment of the full length sequence referred to, more specifically one or more amino acid or nucleic acid sequence which is, relative to the full-length sequence, truncated at one or both termini by one or more amino acids. Such a fragment comprises or encodes for a peptide having at least 6, 7, 8, 10, 12, 15, 20, 25, 50, 75, 100, 150, 200, 300, 400, 500, 600, 700, 800, 900, 1000, 1100, 1200, 1300, 1400, 1500, 1600 or 1650 successive amino acids of the original sequence or a variant thereof. The total length of the variant may be at least 6, 7, 8, 9, 10, 11, 12, 20, 25, 30, 40, 50, 60, 70, 80, 90, 100, 200, 300, 400, 500, 600, 700, 800, 900, 1000, 1100, 1200, 1300, 1400, 1500, 1600, 1650 or 1668 or more amino acids.

In a preferred embodiment, the term “variant”, as used herein, may refer to the full length sequence or at least one fragment of the full length sequence referred to with an additional tag selected from the group of tags comprising His, 18A, ACP, Aldehyd, Avi, BCCP, Calmodulin, Chitin binding protein, E-Tag, ELK16, FLAG, flash, poly glutamate, poly aspartate, GST, GFP, HA, Isope, maltose binding protein, myc, nus, NE, ProtA, ProtC, Thold4, S-Tag, SnoopTag, SpyTag, SofTag, Streptavidin, Strep-tag II, T7 Epitope Tag, TAP, TC, Thioredoxin, Ty, V5, VSV and Xpress Tag.

The term “variant” relates not only to at least one fragment, but also to a polypeptide or a fragment thereof comprising amino acid sequences that are at least 40, 50, 60, 70, 75, 80, 85, 90, 92, 94, 95, 96, 97, 98, 99, 99,3, 99,4, 99,5, 99,6, 99,7, 99,8 or 99,9, preferably at least 99,3% identical to the reference amino acid sequence referred to or the fragment thereof, wherein amino acids other than those essential for the biological activity, for example the ability of an antigen to bind to an (auto)antibody, or the fold or structure of the polypeptide are deleted or substituted and/or one or more such essential amino acids are replaced in a conservative manner and/or amino acids are added such that the biological activity of the polypeptide is preserved. The state of the art comprises various methods that may be used to align two given nucleic acid or amino acid sequences and to calculate the degree of identity, see for example Arthur Lesk (2008), Introduction to bioinformatics, Oxford University Press, 2008, 3^{rd} edition. In a preferred embodiment, the ClustalW software (Larkin, M. A., Blackshields. G., Brown, N. P., Chenna, R., McGettigan, P. A., McWilliam, H., Valentin, F., Wallace, I. M., Wilm, A., Lopez, R., Thompson, J. D., Gibson, T. J., Higgins, D. G. (2007). Clustal W and Clustal X version 2.0. Bioinformatics, 23, 2947-2948) is used using default setting.

In a preferred embodiment, the polypeptide and variants thereof may, in addition, comprise chemical modifications, for example isotopic labels or covalent modifications such as glycosylation, phosphorylation, acetylation, decarboxylation, citrullination, methylation, hydroxylation and the like. The person skilled in the art is familiar with methods to modify polypeptides. Any modification is designed such that it does not abolish the biological activity of the variant.

Moreover, variants may also be generated by N- or/and C-terminal fusion of polypeptides, fragments or variants thereof with other known polypeptides or variants thereof and comprise active portions or domains, preferably having a sequence identity of at least 70, 75, 80, 85, 90, 92, 94, 95, 96, 97, 98 or 99% when aligned with the active portion of the reference sequence, wherein the term “active portion”, as used herein, refers to an amino acid sequence, which is less than the full length amino acid sequence or, in the case of a nucleic acid sequence, codes for less than the full length amino acid sequence, respectively, and/or is a variant of the natural sequence, but retains at least some of the biological activity. Preferably the active portion is an active portion having sequence SEQ ID NO: 10 or a variant thereof, having sequence SEQ ID NO: 23 or a variant thereof or having sequence SEQ ID NO: 82 or a variant thereof. Preferably the active portion is an active portion having sequence SEQ ID NO: 86, SEQ ID NO: 87, SEQ ID NO: 88. SEQ ID NO: 89, SEQ ID NO: 90, SEQ ID NO: 91, SEQ ID NO: 92 or SEQ ID NO: 93 or a variant thereof.

In a preferred embodiment of the present invention, a variant of SEQ ID NO: 1 refers to a sequence according to SEQ ID NO: 35, SEQ ID NO: 38, SEQ ID NO: 41, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 86, SEQ ID NO: 87, SEQ ID NO: 88, SEQ ID NO: 89, SEQ ID NO: 90, SEQ ID NO: 91, SEQ ID NO: 92 and SEQ ID NO: 93.

The variant of the polypeptide has biological activity. In a preferred embodiment, such biological activity is the ability to bind specifically to an autoantibody binding specifically to the autoantigen of interest, preferably the autoantigen having the sequence of SEQ ID NO: 1, SEQ ID NO: 11, SEQ ID NO: 24, SEQ ID NO: 10, SEQ ID NO: 23 or SEQ ID NO: 82 or a variant thereof as found preferably in a patient suffering from an autoimmune disease associated with such autoantibody, preferably SARD, more preferably SSc. For example, whether or not a variant of the polypeptide has such biological activity may be checked by determining whether or not it binds specifically to an autoantibody from a sample of a SARD, preferably of a SSc patient comprising an autoantibody binding specifically to wild type autoantigen, preferably as determined by indirect immunofluorescence.

According to the present invention, a polypeptide having the sequence SEQ ID NO: 1, SEQ ID NO: 11 or SEQ ID NO: 24 or a variant thereof may be a recombinant protein. In a preferred embodiment, the term “recombinant”, as used herein, refers to a polypeptide produced using genetic engineering approaches at any stage of the production process, for example by fusing a nucleic acid encoding the polypeptide to a strong promoter for overexpression in cells or tissues or by engineering the sequence of the polypeptide itself. The person skilled in the art is familiar with methods for engineering nucleic acids and polypeptides encoded (for example, described in Sambrook, J., Fritsch, E. F. and Maniatis, T. (1989), Molecular Cloning, CSH or in Brown T. A. (1986), Gene Cloning—an introduction, Chapman & Hall) and for producing and purifying native or recombinant polypeptides (for example Handbooks “Strategies for Protein Purification”, “Antibody Purification”, published by GE Healthcare Life Sciences, and in Burgess, R. R., Deutscher, M. P. (2009): Guide to Protein Purification). In another preferred embodiment, a polypeptide provided or used according to the present invention such as a polypeptide having the sequence SEQ ID NO: 1, SEQ ID NO: 11 or SEQ ID NO: 24 or a variant thereof or an autoantibody according to the present invention is an isolated polypeptide, wherein the term “isolated” means that the polypeptide has been enriched compared to its state upon production using a biotechnological or synthetic approach and is preferably pure, i.e. at least 60, 70, 80, 90, 95 or 99 percent of the polypeptide in the respective liquid consists of said polypeptide as judged by SDS polyacrylamide gel electrophoresis followed by Coomassie blue staining and visual inspection. Preferably any polypeptide on a carrier used as a means to capture an antibody is pure.

According to the present invention, a medical or diagnostic device such as the diagnostically useful carrier may be prepared by expressing a recombinant variant of a polypeptide having the sequence SEQ ID NO: 1, SEQ ID NO: 11 or SEQ ID NO: 24 or a variant thereof, comprising an affinity tag, optionally with an artificial linker which may include a protease cleavage site, in a cell such as a eukaryotic or prokaryotic cell, contacting the expressed variant with a ligand binding specifically to the affinity tag, which ligand is immobilized on a solid phase, washing the solid phase such that non-specifically bound material from the cell is removed and eluting the expressed variant from the solid phase, preferably by adding an excess of non-immobilized ligand. The variant may then be immobilized on the device. Optionally, the affinity tag may be removed by contacting the variant with a protease, preferably a protease recognizing the protease cleavage site, before the immobilization. The affinity tag may be selected from the group of tags comprising His, 18A, ACP, Aldehyd, Avi, BCCP, Calmodulin, Chitin binding protein, E-Tag, ELK16, FLAG, flash, poly glutamate, poly aspartate, GST, GFP, HA, Isope, maltose binding protein, myc, nus, NE, ProtA, ProtC, Thold4, S-Tag, SnoopTag, SpyTag, SofTag, Streptavidin, Strep-tag II, T7 Epitope Tag, TAP, TC, Thioredoxin, Ty, V5, VSV and Xpress Tag. Useful proteases include, but are not limited to TEV, Thrombin, factor Xa or Enteropeptidase. Suitable linkers are part of vectors, for example pET vector series (Novagen).

According to the present invention, a cell is provided which overexpresses a polypeptide having the sequence SEQ ID NO: 1, SEQ ID NO: 11 or SEQ ID NO: 24 or a variant thereof, preferably in combination with another cell which overexpresses the sequence of another SARD-related autoantigen in addition or a variant thereof. Another SARD-related autoantigen herein is preferably selected from the group comprising dsDNA, nucleosomes, histones, SS-A, Ro-52, SSB, RNP/Sm, Sm, Mi-2α, Mi-2β, Ku, CENP A, CENP B, Sp100, PML, Scl-70, PM100, PM75, RP11, RP155, gp210, PCNA, Fibrillarin, NOR90, Th/To, PDGFR and DSF70. In a preferred embodiment, the term “overexpressing”, as used herein, means that the cell has been transfected with a nucleic acid, either transiently or stably in the sense that the nucleic acid has been incorporated in the genome of the cell, that comprises a nucleic acid sequence encoding a polypeptide having the sequence SEQ ID NO: 1, SEQ ID NO: 11 or SEQ ID NO: 24 or a variant thereof or another SARD-related autoantigen or a variant thereof under the control of a promotor. The cell overexpressing a polypeptide having the sequence SEQ ID NO: 1, SEQ ID NO: 11 or SEQ ID NO: 24 or a variant thereof may additionally overexpress a polypeptide to another SARD-related autoantigen or a variant thereof. Consequently, the transfected cell expresses more polypeptide recognized by the autoantibody to be detected than the same type of cell normally would, probably at least 10, 20, 30, 50, 100, 200 or 500% more as judged by quantitative Western Blot. The promotor may be an inducible promotor, which allows for the induction of expression by addition of an inducer. The person skilled in the art is familiar with protocols and vectors for transiently overexpressing a polypeptide in a eukaryotic cell, for example the pTriEx system from Novagen and with protocols and vectors for stably transfecting a eukaryotic cell, for example the pcDNA™4/TO vector system from Invitrogen.

In a preferred embodiment, a fixed mammalian cell may be used. In a preferred embodiment, the term “fixed” cell, as used herein, refers to a cell that has been treated with a reactive chemical compound to the effect that the cell is no longer metabolically active, but still presents its epitopes for immunostaining with antibodies and their subsequent detection, for example by fluorescence. More preferably, the reactive chemical compound is selected from the group comprising acetone, formalin, methanol and ethanol or mixtures thereof, preferably all of them. The person skilled in the art is familiar with protocols that may be used to prepare fixed cells. Essentially, the cell which is attached to a solid support is washed by using washing buffer, followed by contacting with the reactive compound, for example immersion. Pure acetone or formalin or aqueous dilutions of the reactive chemical compound may be used.

According to the present invention, the cell is on a carrier for microscopic immunofluorescence analysis. Such a carrier may be a glass slide. The cell on the glass slide may be covered with a mounting buffer. A mounting medium is a liquid which helps maintain a near physiological pH to maintain the molecular structure of any diagnostically relevant molecular and their epitopes, is compatible with the emission of a fluorescence signal and prevents a premature loss of fluorescence due to bleaching of the fluorophore. At the same time its optical properties are matched with other buffers used, in particular its refractive index which allows for an efficient microscopic fluorescence analysis. The mounting medium comprises a base component, preferably selected from the group comprising water, glycerol, natural oil or plastic or a mixture thereof, preferably water and glycerol. It may further comprise an antifade constituent which may reduce bleaching, preferably selected from the group comprising NPG (N-propyl gallate), DABCO (1,4-diazabicyclo[2.2.2]octane), 4POBN ((4-Pirydyl-1-oxide)-N-tert-butyl nitrone) and PPD (P-phenylanediamine). Various compositions and methods are described in the state of the art, for example in “Mountants and Antifades”, published by Wright Cell Imaging Facility, Toronto Western Research Institute University Health Network, (https://de.scribd.com/document/47879592/Mountants-Antifades), Krenek et al. (1989) Comparison of antifading agents used in immunofluorescence, J. Immunol. Meth 117, 91-97 and Naim et al. (1969) Microphotometry in Immunofluorescence. Clin. Exp. Immunol. 4, 697-705.

A cover glass may be placed on top of the composition comprising the sample and the mounting medium. Slides with cover glasses (FB 112d-1005-1 or ZZ 3000-0112) are available from EUROIMMUN Medizinische Labordiagnostika AG, Lübeck, Germany. However, any carrier compatible with microscopic analysis of the fluorescence pattern may be used. The carrier may comprise a mock-transfected cell, which has been transfected with the same vector as the cell overexpressing a polypeptide having the sequence SEQ ID NO: 1, SEQ ID NO: 11 or SEQ ID NO: 24 or a variant thereof, but without the nucleic acid encoding for the latter. Such mock-transfected cell may serve as a negative control. The carrier is configured for analysis using an immunofluorescence microscope.

In a preferred embodiment, the carrier may comprise a field comprising the cell according to the invention. In addition the carrier may comprise additional fields. The fields are preferably surrounded by a hydrophobic surface. Each of these fields may comprise a cell overexpressing another SARD-related autoantigen or a variant thereof. A field may comprise a section of mammalian kidney or liver tissue, preferably primate.

Preferably a polypeptide having the sequence SEQ ID NO: 1, SEQ ID NO: 11 or SEQ ID NO: 24 or a variant thereof is immobilized on a solid phase of the carrier. It may be directly immobilized on the solid phase when contacted with the sample, but a competitive assay, a capture bridge assay, an immunometric assay, a class-specific second antibody on the solid phase, a class capture assay, direct or indirect may also be used. The principle of each of these formats is detailed in The Immunoassay Handbook, 3′ edition, edited by David Wild, Elsevier, 2005. More preferably, the solid phase is a test strip or a well of a microtiter plate for ELISA, preferably a well of a microtiter plate for ELISA.

In a preferred embodiment, a secondary antibody is an antibody binding specifically to all antibodies from an antibody class, preferably a mammalian antibody class, more preferably human antibody class such as IgG. Secondary antibodies typically recognize the constant domain of said class, but may also recognize other epitopes shared by antibodies from the class of interest, for example a conformational epitope across the 3D structure. A wide range of them is commercially available, for example from Thermo Fisher. It may be a monoclonal or a polyclonal antibody. In a preferred embodiment, the term “recognized”, as used herein, means that the secondary antibody binds specifically to the antibody or antibodies to be detected. A secondary antibody may bind specifically to all isotypes from the antibody class. For example a secondary antibody to IgG class antibodies may bind to IgG1, IgG2, IgG3 and IgG4 isotypes. This may be achieved by using as a secondary antibody to the class, preferably to IgG class antibodies, a mixture comprising an antibody binding specifically to each IgG isotype or a single antibody which reacts with all isotypes of interest. The use of secondary antibodies is explained in Kruger, N. J., Detection of Polypeptides on Blots Using Secondary Antibodies, in The Protein Protocols Handbook (ed. J. M. Walker), page 967, volume 1996, Springer. Briefly, such secondary antibodies may be generated by immunizing a laboratory animal with the antibody to be recognized or a mixture of the antibodies to be recognized.

The autoantibody to be detected or a secondary antibody used binds preferably specifically to the autoantigen or antibody to be detected, respectively. In a preferred embodiment, the term “binding specifically”, as used herein, preferably means that the binding reaction is stronger than a binding reaction characterized by a dissociation constant of 1×10⁻⁵ M, more preferably 1×10⁻⁷ M, more preferably 1×10⁻⁸ M, more preferably 1×10⁻⁹ M, more preferably 1×10⁻¹⁰ M, more preferably 1×10⁻¹¹ M, more preferably 1×10⁻¹² M, as determined by surface plasmon resonance using Biacore equipment at 25° C. in PBS buffer at pH 7.

In a preferred embodiment, the cell is bound to an autoantibody to a polypeptide having the sequence SEQ ID NO: 1, SEQ ID NO: 11 or SEQ ID NO: 24 or a variant thereof, and a secondary antibody is bound to the autoantibody. In a more preferred embodiment, the secondary antibody recognizes IgG class antibodies. For immunofluorescence analysis, the secondary antibody may comprise a detectable fluorescent label, more preferably FITC (fluorescein isothiocyanate).

In a preferred embodiment, the method according to the present invention comprises the step providing the carrier according to the present invention. The carrier may then be contacted with the sample suspected of comprising the autoantibody under conditions allowing for binding of any autoantibodies to the cell and the polypeptide having the sequence SEQ ID NO: 1, SEQ ID NO: 11 or SEQ ID NO: 24 or a variant thereof expressed by the cell. The sample may then be removed and the carrier with the cell may be washed to remove any remaining sample. A secondary autoantibody or similar reagent or means binding to the autoantibody and carrying a detectable label such as a fluorescent dye may then be contacted with the carrier under conditions allowing formation of a complex between any bound autoantibody and the secondary antibody. The carrier may be washed then to remove non-bound secondary antibody. Finally, the presence of the autoantibody is detected by checking whether the secondary antibody may be detected, preferably by immunofluorescence, more preferably emitted by fluorescein or a derivative thereof, most preferably FITC.

In a preferred embodiment, the term “diagnosis”, as used herein, is to be used in its broadest possible sense and may to any kind of procedure aiming to obtain information instrumental in the assessment whether a patient, known or an anonymous subject from a cohort, suffers or is likely or more likely than the average or a comparative subject, the latter preferably having similar symptoms, to suffer from certain a disease or disorder in the past, at the time of the diagnosis or in the future, to find out how the disease is progressing or is likely to progress in the future or to evaluate the responsiveness of a patient or patients in general with regard to a certain treatment, for example the administration of immunosuppressive drugs, or to find out whether a sample is from such a patient. Such information may be used for a clinical diagnosis, but may also be obtained by an experimental and/or research laboratory for the purpose of general research, for example to determine the proportion of subjects suffering from the disease in a patient cohort or in a population. In other words, the term “diagnosis” comprises not only diagnosing, but also prognosticating and/or monitoring the course of a disease or disorder, including monitoring the response of one or more patients to the administration of a drug or candidate drug, for example to determine its efficacy. While the result may be assigned to a specific patient for clinical diagnostic applications and may be communicated to a medical doctor or institution treating said patient, this is not necessarily the case for other applications, for example in diagnostics for research purposes, where it may be sufficient to assign the results to a sample from an anonymized patient. In another preferred embodiment, the detection of an autoantibody to a polypeptide having the sequence SEQ ID NO: 1, SEQ ID NO: 11 or SEQ ID NO: 24 or a variant thereof is considered to imply a definitive diagnosis of SARD, preferably SSc, because of the presence of the autoantibody.

In a preferred embodiment, the methods and products according to the present invention may be used for interaction studies, including determining whether a drug candidate or other compound may interfere with the binding of an autoantibody to a polypeptide having the sequence SEQ ID NO: 1, SEQ ID NO: 11 or SEQ ID NO: 24 or a variant thereof or may affect any downstream process or the strength of its binding to its target. In a preferred embodiment, they may be used for monitoring the immune response, more preferably the emergence and/or titer of antibodies to a polypeptide having the sequence SEQ ID NO: 1, SEQ ID NO: 11 or SEQ ID NO: 24 or a variant thereof, following the administration of an immunogenic composition comprising a polypeptide having the sequence SEQ ID NO: 1, SEQ ID NO: 11 or SEQ ID NO: 24 or a variant thereof or an immunogenic variant thereof, for example to a mammal, which may be a mammal other than a human such as a laboratory animal.

In another preferred embodiment, the methods and products according to the present invention may be used for determining the concentration of an antibody to a polypeptide having the sequence SEQ ID NO: 1, SEQ ID NO: 11 or SEQ ID NO: 24 or a variant thereof. In a more preferred embodiment, said antibody is an autoantibody from a SARD patient, preferably from a SSc patient. In another preferred embodiment, said antibody is a recombinant antibody which binds to a polypeptide having the sequence SEQ ID NO: 1, SEQ ID NO: 11 or SEQ ID NO: 24 or a variant thereof, but is recognized by a secondary antibody binding specifically to human IgG class antibodies, preferably IgG1, IgG2, IgG3 and IgG4 isotypes. In a more preferred embodiment, such a concentration needs to be determined for the purposes of research, for the preparation or for monitoring the quality of reagents, animal models or devices that may or may not be used for the diagnosis of SARD, preferably SSc.

In many cases the mere detection of the autoantibody, in other words determining whether or not detectable levels of the antibody are present in the sample, is sufficient for the diagnosis. If the autoantibody can be detected, this will be information instrumental for the clinician's diagnosis and indicates an increased likelihood that the patient suffers from a disease.

The person skilled in the art will appreciate that a clinician does usually not conclude whether or not the patient suffers or is likely to suffer from a disease, condition or disorders solely on the basis of a single diagnostic parameter, but needs to take into account other aspects, for example the presence of other autoantibodies, markers, blood parameters, clinical assessment of the patient's symptoms or the results of medical imaging or other non-invasive methods such as polysomnography, to arrive at a conclusive diagnosis. See Baenkler H. W. (2012), General aspects of autoimmune diagnostics, in Renz, H., Autoimmune diagnostics, 2012, de Gruyter, page 3. The value of a diagnostic agent or method may also be the possibility to rule out one disease, thus allowing for the indirect diagnosis of another. In a preferred embodiment, the meaning of any symptoms or diseases referred to throughout this application is in line with the person skilled in the art's understanding as of the filing date or, preferably, earliest priority date of this application as evidenced by textbooks and scientific publications. It should be mentioned that the inventive methods or uses or products, taken alone, cannot be used to arrive at a definite, final diagnosis.

In a preferred embodiment, the term “diagnosis” may also refer to a method or agent used to choose the most promising treatment regime for a patient. In other words, the method or agent may relate to selecting a treatment regimen for a subject. For example, the detection of autoantibodies may indicate that an immunosuppressive therapy is to be selected, which may include administrating to the patient one or more immunosuppressive drugs.

In a preferred embodiment, any information or data demonstrating the presence of absence of the autoantibody may be communicated to the patient or a medical doctor treating the patient, preferably by telephone, by fax, in a written form or via the internet, for example as an email or text message.

In a preferred embodiment, the term “autoantibody”, as used herein, refers to an antibody binding specifically to an endogenous molecule of the animal, preferably mammal, more preferably human, which produces said autoantibody, wherein the level of such antibody is more preferably elevated compared to the average healthy subject. The autoantibody may have the sequence of an antibody's constant regions from the animal, preferably human, making it, but the variable region is able to bind specifically to the endogenous molecule of the animal, more specifically to a polypeptide having the sequence SEQ ID NO: 1, SEQ ID NO: 11 or SEQ ID NO: 24 or a variant thereof or a SARD-related, preferably SSc-related autoantigen. In a preferred embodiment, the autoantibody is isolated and/or purified from a sample, preferably tissue, serum, plasma, blood or CSF from the animal, preferably human. The autoantibody is a polyclonal, native antibody from the animal rather than a synthetic or recombinant antibody.

The method according to the present invention is preferably an in vitro method.

According to the present invention, a kit is provided, comprising the cell or the carrier and further comprising one or more, preferably all reagents from the group comprising a secondary antibody, preferably labeled with a detectable label, a washing solution, a positive control, a negative control, a detergent, a cover glass, a mounting medium and a physiological salt solution, preferably PBS, or salt required to prepare it. In a preferred embodiment, the positive control is a diluted sample, preferably serum or CSF, from a patient suffering from SARD, preferably SSc or a monoclonal antibody to a polypeptide having the sequence SEQ ID NO: 1, SEQ ID NO: 11 or SEQ ID NO: 24 or a variant thereof. The negative control may be a diluted sample from a healthy subject, for example a blood donor. The kit may comprise instructions how to carry out the assay. Preferably, the secondary antibody is a secondary antibody to IgG class antibodies, preferably human IgG class antibodies.

In a preferred embodiment, the present invention provides a use of the cell, the polypeptide, the carrier for the manufacture of kit a composition for the diagnosis of a disease.

In a preferred embodiment, any method or use according to the present invention may be intended for a non-diagnostic use, i.e. determining the presence of an autoantibody to binding to a polypeptide having the sequence SEQ ID NO: 1, SEQ ID NO: 11 or SEQ ID NO: 24 or a variant thereof, for a use other than diagnosing a patient. For example, the method or use may be for testing in vitro the efficiency of a medical device designed to remove an autoantibody from a patient's blood, wherein the testing is performed on a liquid other than patient's blood. After the use of the medical device with a patient, its capacity to remove autoantibody may be checked by running a solution comprising antibody to a polypeptide having the sequence SEQ ID NO: 1, SEQ ID NO: 11 or SEQ ID NO: 24 or a variant thereof through the device, followed by use of the method according to the present invention to confirm that less or no antibody is in the solution that has been passed through the device, i.e. showing that the device has still the capacity to remove antibody from the solution.

In another preferred embodiment, the method may be for confirming the reliability of a diagnostic assay and may involve detecting an antibody to a polypeptide having the sequence SEQ ID NO: 1, SEQ ID NO: 11 or SEQ ID NO: 24 or a variant thereof in a solution, which is not a sample from a patient who requires a diagnosis, but is known to comprise an antibody to a polypeptide having the sequence SEQ ID NO: 1, SEQ ID NO: 11 or SEQ ID NO: 24 or a variant thereof, preferably at a known concentration. For example, it may be a recombinant antibody or a sample diluted in a dilution buffer such as PBS from an anonymous patient whose identity cannot be traced back. Alternatively, the solution may be a negative control not comprising the antibody to check the background. Such method may be run in parallel with, after or before a diagnostic method. In a preferred embodiment, any method or use according to the present invention may be intended for generating an autoantibody profile, preferably for detecting a disease in a mammal, preferably a human.

In a preferred embodiment, any method or use according to the present invention may be for identifying a subject at risk of suffering from or developing a disease, preferably SARD, more preferably SSc, and/or a tumor.

In a preferred embodiment, the method may be for detecting an antibody, preferably autoantibody in a solution which is not a sample from a mammal to be diagnosed or for the purpose of providing a diagnosis, in particular not a diagnosis of SARD.

In a preferred embodiment, the present invention provides an apparatus for analyzing a sample from a patient to detect an autoantibody against a polypeptide having the sequence SEQ ID NO: 1, SEQ ID NO: 11 or SEQ ID NO: 24 or a variant thereof, indicating an increased likelihood of a disease or of developing a disease, comprising:
a. a carrier, which contains a means for capturing the autoantibody from the sample when the sample is contacted with the carrier, wherein the means is the cell and the carrier is the carrier according to the present invention,
b. a detectable means capable of binding to the antibody captured by the carrier when the detectable means is contacted with the carrier, wherein the detectable means is preferably a labeled secondary antibody capable of binding to the autoantibody captured on the carrier,
c. optionally a means for removing any sample from the carrier and the detectable means, preferably by washing;
d. a detecting device for detecting the presence of the detectable means and converting the results into an electrical signal, for example a fluorescence reader or a fluorescence microscope connected with a software capable of recognizing a pattern characteristic of a stained cell overexpressing a polypeptide having the sequence SEQ ID NO: 1, SEQ ID NO: 11 or SEQ ID NO: 24 or a variant thereof in an image of the cell taken by the fluorescence reader or camera, and

optionally a means for receiving the electronical signal from the detecting device and determining if the level of the signal is indicative of an increased likelihood of having or developing a disease, by comparing with the patterns characteristic of wild type or non-stained cells, preferably by a mock-transfected cell or cells not positively stained by an autoantibody binding specifically to a polypeptide having the sequence SEQ ID NO: 1, SEQ ID NO: 11 or SEQ ID NO: 24 or a variant thereof on the same carrier, or an input reference value obtained with samples from healthy subjects or by comparing the level of signal obtained with one sample with the level of signal obtained with a second sample obtained at a later time point, preferably at least one month later.

According to the present invention, a device for removing an autoantibody to a polypeptide having the sequence SEQ ID NO: 1, SEQ ID NO: 11 or SEQ ID NO: 24 or a variant thereof, from blood, preferably serum of a SARD patient, preferably of an SSc patient, wherein the device comprises a carrier coated with a polypeptide having the sequence SEQ ID NO: 1, SEQ ID NO: 11 or SEQ ID NO: 24 or a variant thereof is provided as an ex vivo method for removing an autoantibody to a polypeptide having the sequence SEQ ID NO: 1, SEQ ID NO: 11 or SEQ ID NO: 24 or a variant thereof, from blood, preferably serum of a SARD patient, preferably of a SSc patient. A device coated with a polypeptide having the sequence SEQ ID NO: 1, SEQ ID NO: 11 or SEQ ID NO: 24 or a variant thereof may be used or a device coated with a secondary antibody or protein capturing all IgG class antibodies, among them IgG class autoantibodies to a polypeptide having the sequence SEQ ID NO: 1, SEQ ID NO: 11 or SEQ ID NO: 24 or a variant thereof. Suitable methods are described in Eisei Noiri and Noria Hanafusa, The Concise Manual of Apharesis Therapy, Springer Tokyo, 2014. Hamilton, P., Kanigicherla, D., Hanumapura, P., Walz, L., Kramer, D., Fischer, M., Brenchley, P., and Mitra, S. (2018) J. Clin. Aph. 33(3), 283-290. Another method is disclosed in EP3477300.

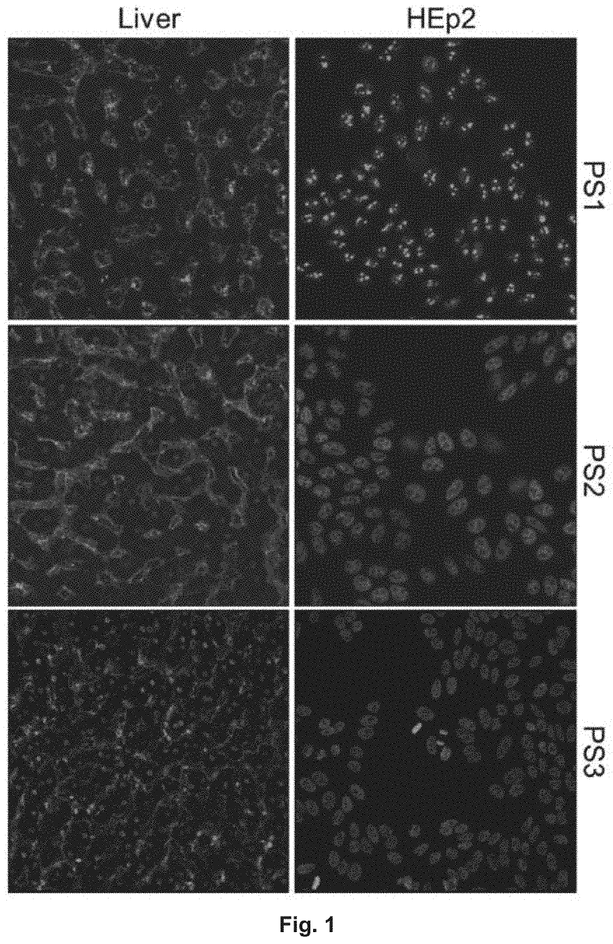

FIG. 1 shows IFA with HEp-2 cells or monkey liver with 1:100 diluted patient sera (PS). Anti-Human-IgG-Alexa488 (1:500) was used as secondary antibody. PS1 showed a nucleolar pattern on both substrates. PS2 and PS3 showed a pseudo-dense fine speckled pattern (magnification 200×).

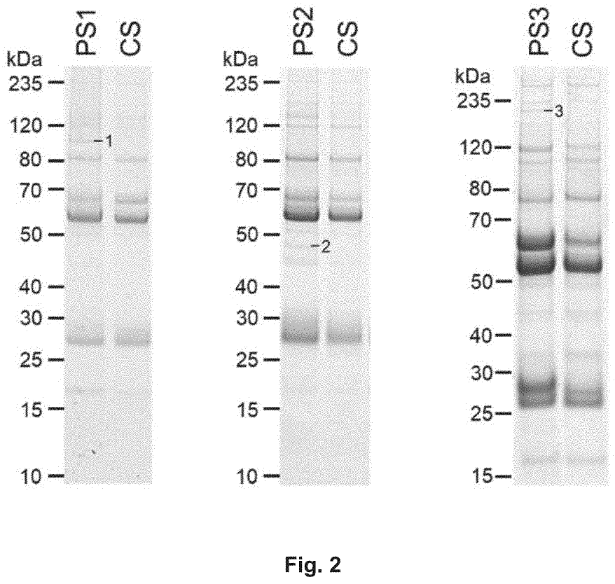

FIG. 2 shows immunoprecipitation of patient (PS1-3) or control sera (CS) with HEp2 extract. Immunoprecipitates were separated by SDS-PAGE, followed by coomassie staining. Selected protein bands were identified as NVL (band 1), CD2BP2 (band 2), or TJP1 (band 3) by MALDI-TOF-MS.

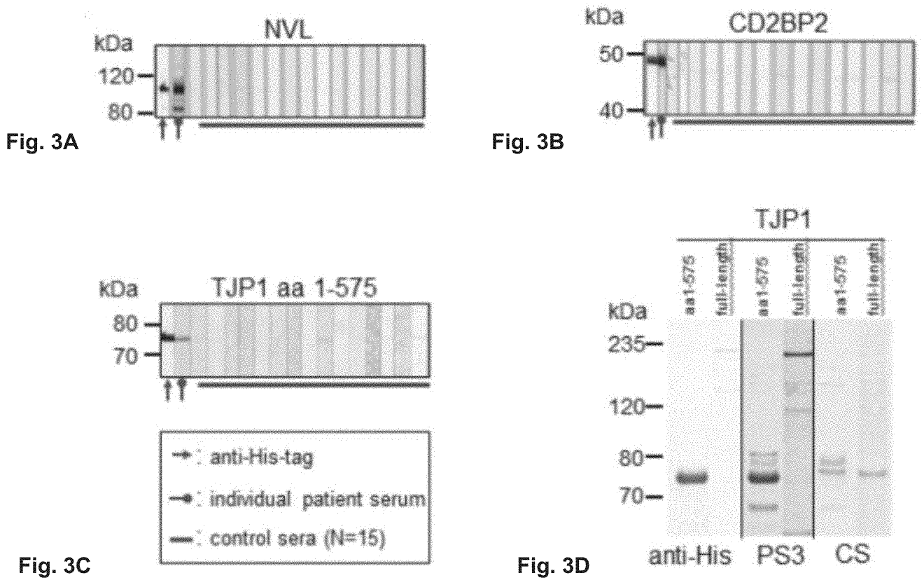

FIG. 3A, FIG. 3B, FIG. 3C, and FIG. 3D show immunoblots with anti-His-tag (1:2000), patient sera (1:200) and healthy control sera (1:200) with recombinant antigens: FIG. 3A) purified NVL-His expressed in E. coli and PS1, FIG. 3B) purified His-CD2BP2 expressed in E. coli and PS2, FIG. 3C) purified His-TJP1 aa1-575 expressed in E. coli and PS3 FIG. 3D) full-length TJP1-His (HEK293 cell lysate) or His-TJP1 aa1-575 (E. coli lysate). Anti-mouse-IgG-AP (1:2000) or anti-human-IgG-B/E-AP (1:10) were used in secondary incubation.

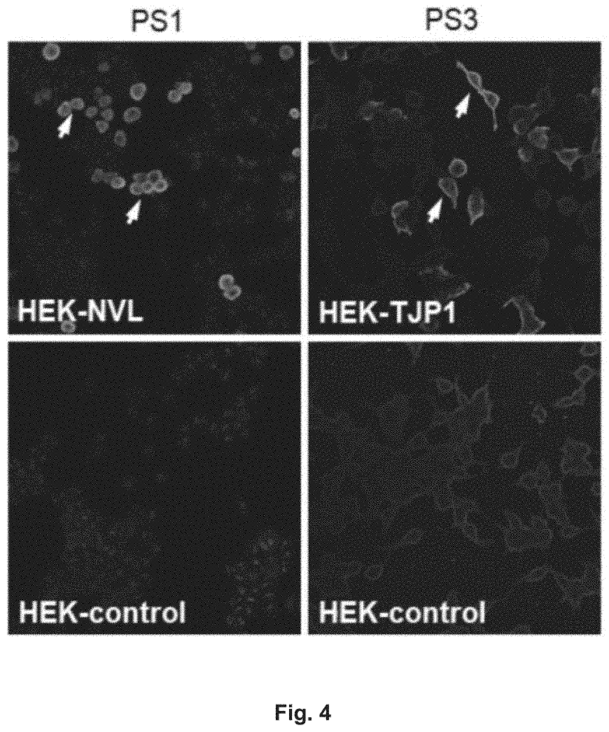

FIG. 4 shows IFA with NVL-His, TJP1-His or control transfected HEK293 cells and PS1 (1:100) or PS3, respectively (1:10). Anti-human-IgG-Alexa488 (1:500) was applied as secondary antibody. Arrows indicate the specific fluorescence signal (magnification 200×).

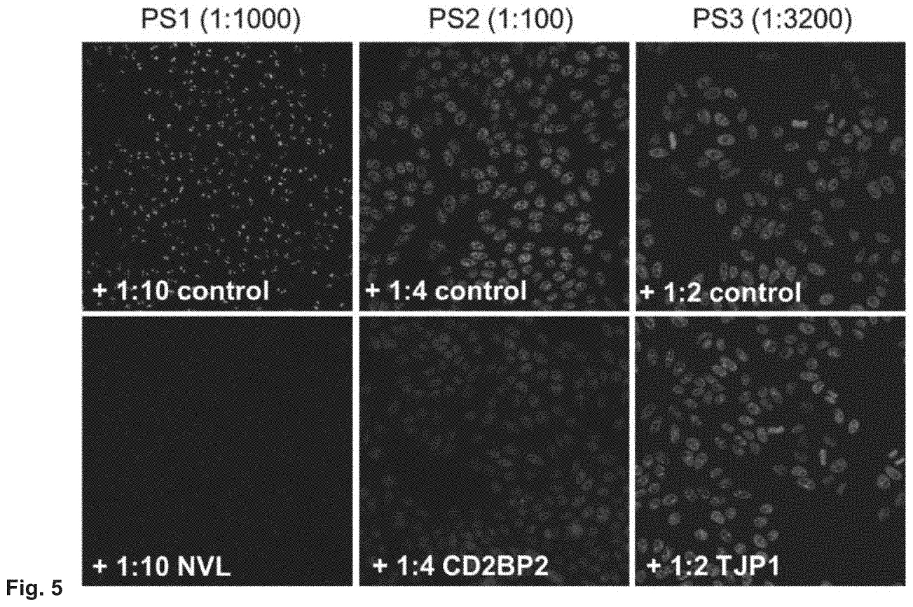

FIG. 5 shows IFA neutralization assay with patient sera and HEp-2 cells. Patient sera were incubated with the diluted purified recombinant antigens or control buffer prior to application in IFA with HEp-2 cells. Individual serum dilutions are indicated. Anti-Human-IgG-Alexa488 (1:500) was used as secondary antibody (magnification 200×).

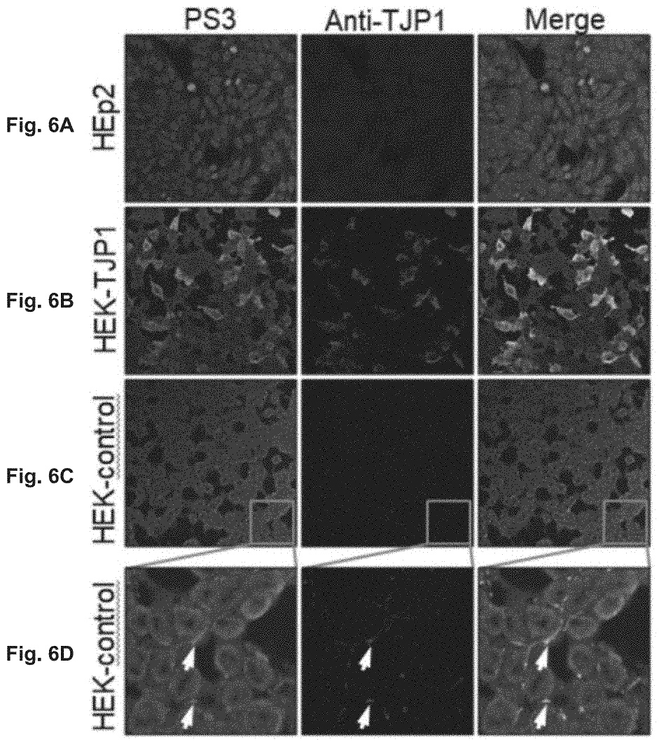

FIG. 6A, FIG. 6B, FIG. 6C, and FIG. 6D show colocalization analysis with anti-TJP1 antibody (mouse, monoclonal) and PS3 in HEp-2, HEK-TJP1 and HEK-control cells. A mixture of PS3 (1:10) and anti-TJP1 (1:50) was applied in IFA. Anti-human-IgG-alexa488 (1:500) and anti-mouse-IgG-Cy3 (1:200) were employed as secondary antibodies. FIG. 6A) HEp-2 cells. FIG. 6B) transfected HEK293 cells overexpressing TJP1-His. FIG. 6C, FIG. 6D) control HEK293 cells transfected with empty vector (FIG. 6A-FIG. 6C magnification 200×, FIG. 6D magnification 3200×).

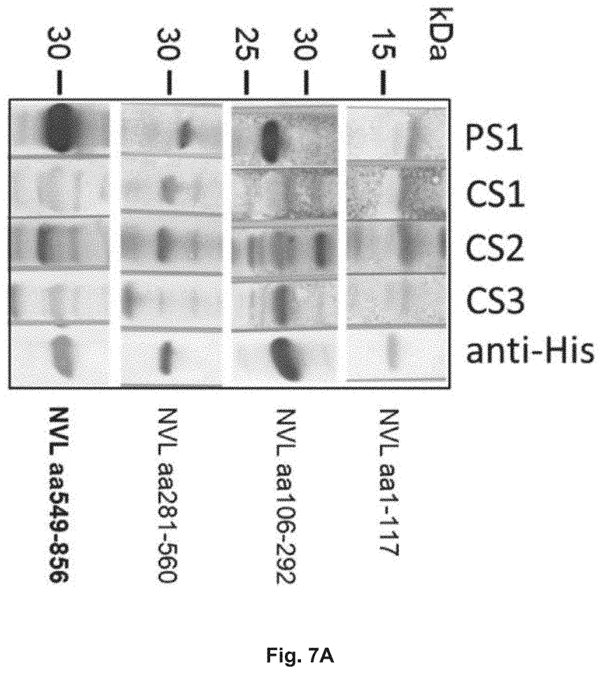

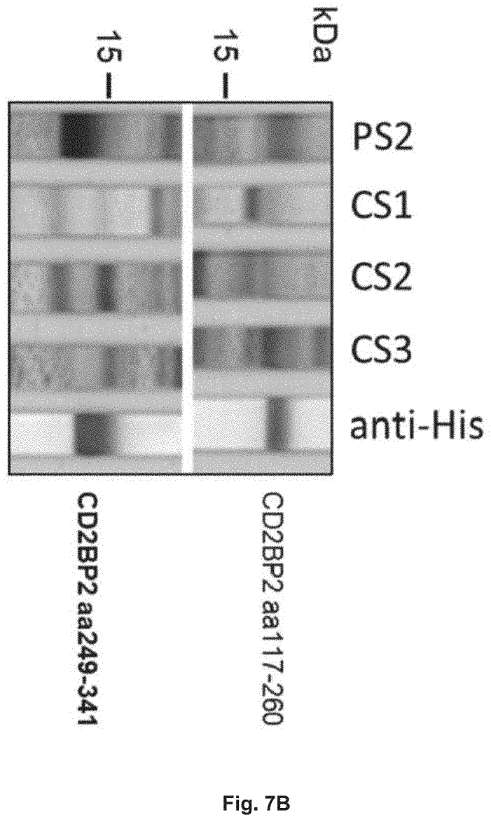

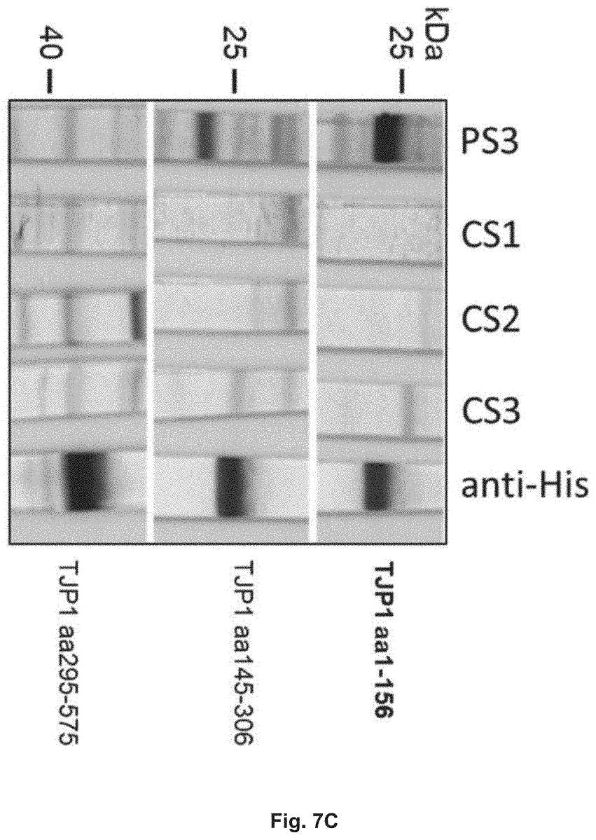

FIG. 7A, FIG. 7B and FIG. 7C show immunoblots with E. coli lysates containing FIG. 7A) NVL fragments (aa1-117, aa106-292, aa281-560, aa549-856) FIG. 7B) CD2BP2 fragments (aa117-260, aa249-341) or FIG. 7C) TJP1 fragments (aa1-156, aa145-306, aa295-575) and respective patient sera (PS1-3, 1:200), control sera (CS1-3, 1:200), or anti-His-tag antibody (1:2000). Anti-mouse-IgG-AP (1:2000) or anti-human-IgG-B/E-AP (1:10) were used in secondary incubation. Fragments with main reactions are indicated by bold letters.

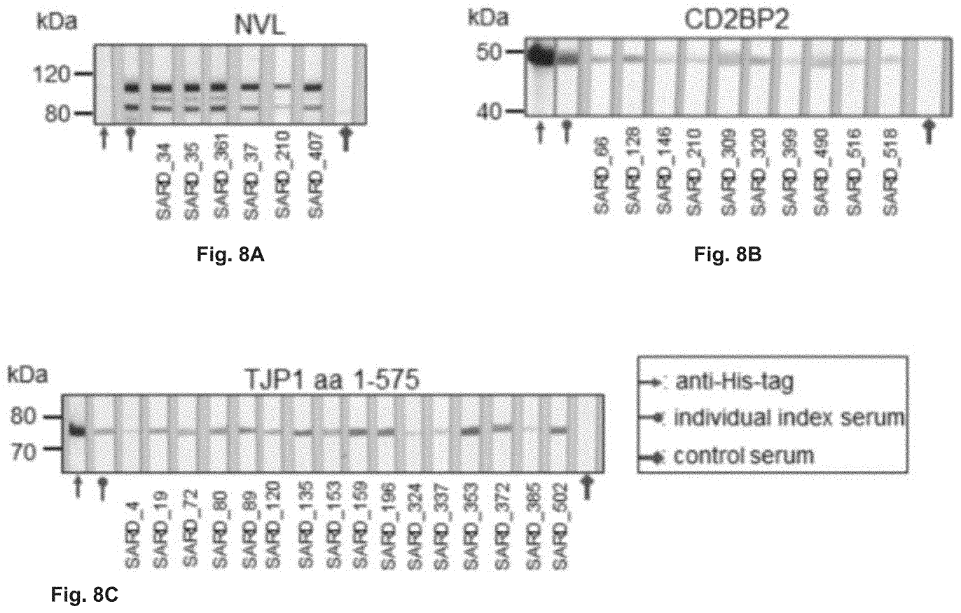

FIG. 8A, FIG. 8B, and FIG. 8C show immunoblots with purified NVL-His (FIG. 8A), His-CD2BP2 (FIG. 8B) or His-TJP1 aa1-575 (FIG. 8C) and anti-NVL, anti-CD2BP2 or anti-TJP1 lineblot positive sera (1:200) from the systemic rheumatic disease (SARD) cohort. As positive and negative controls anti-His-tag-AP (1:8000), PS1-3 (1:200) and control sera (1:200) were incubated in parallel. Anti-human-IgG-B/E-AP (1:10) was used in secondary incubation.

Sequences:

The present invention comprises a range of polypeptides. The exact sequences are provided with the sequence listing if not disclosed in the following:

[NVL-isoform1, Uniprot O15381-1]
SEQ ID NO: 1
MKPRPAGFVDNKLKQRVIQYLTSNKCGKYVDIGVLASDLQRVYSIDYGRRKRNAFRIQVEKV
FSIISSEKELKNLTELEDEHLAKRARQGEEDNEYTESYSDDDSSMEDYPDPQSANHMNSSL
LSLYRKGNPDSVSNTPEMEQRETTSSTPRISSKTGSIPLKTPAKDSEGGWFIDKTPSVKKDS
FFLDLSCEKSNPKKPITEIQDSKDSSLLESDMKRKGKLKNKGSKRKKEDLQEVDGEIEAVLQ
KKAKARGLEFQISNVKFEDVGGNDMTLKEVCKMLIHMRHPEVYHHLGVVPPRGVLLHGPPG
CGKTLLAHAIAGELDLPILKVAAPEIVSGVSGESEQKLRELFEQAVSNAPCIIFIDEIDAITPKRE
VASKDMERRIVAQLLTCMDDLNNVAATARVLVIGATNRPDSLDPALRRAGRFDREICLGIPD
EASRERILQTLCRKLRLPQAFDFCHLAHLTPGFVGADLMALCREAAMCAVNRVLMKLQEQQ
KKNPEMEDLPSKGVQEERLGTEPTSETQDELQRLLGLLRDQDPLSEEQMQGLCIELNDFIV
ALSSVQPSAKREGFVTVPNVTWADIGALEDIREELTMAILAPVRNPDQFKALGLVTPAGVLLA
GPPGCGKTLLAKAVANESGLNFISVKGPELLNMYVGESERAVRQVFQRAKNSAPCVIFFDE
VDALCPRRSDRETGASVRVVNQLLTEMDGLEARQQVFIMAATNRPDIIDPAILRPGRLDKTL
FVGLPPPADRLAILKTITKNGTKPPLDADVNLEAIAGDLRCDCYTGADLSALVREASICALRQ
EMARQKSGNEKGELKVSHKHFEEAFKKVRSSISKKDQIMYERLQESLSR
[NVL-isoform3]
SEQ ID NO: 2
[NVL gene synthesis]
SEQ ID NO: 3
[sense NVL-IF1]
SEQ ID NO: 4
[asense NVL-IF1]
SEQ ID NO: 5
[NVL-PCR-fragment]
SEQ ID NO: 6
[pTriEx-1-NVL[human]-IF1-H8]
SEQ ID NO: 7
[NVL[human]-IF1-H8]
SEQ ID NO: 8
[pET24d-NVL[human]-IF1-H6]
SEQ ID NO: 9
[NVL[human]-IF1-H6]
SEQ ID NO: 10
MKPRPAGFVDNKLKQRVIQYLTSNKCGKYVDIGVLASDLQRVYSIDYGRRKRNAFRIQVEKV
FSIISSEKELKNLTELEDEHLAKRARQGEEDNEYTESYSDDDSSMEDYPDPQSANHMNSSL
LSLYRKGNPDSVSNTPEMEQRETTSSTPRISSKTGSIPLKTPAKDSEGGWFIDKTPSVKKDS
FFLDLSCEKSNPKKPITEIQDSKDSSLLESDMKRKGKLKNKGSKRKKEDLQEVDGEIEAVLQ
KKAKARGLEFQISNVKFEDVGGNDMTLKEVCKMLIHMRHPEVYHHLGVVPPRGVLLHGPPG
CGKTLLAHAIAGELDLPILKVAAPEIVSGVSGESEQKLRELFEQAVSNAPCIIFIDEIDAITPKRE
VASKDMERRIVAQLLTCMDDLNNVAATARVLVIGATNRPDSLDPALRRAGRFDREICLGIPD
EASRERILQTLCRKLRLPQAFDFCHLAHLTPGFVGADLMALCREAAMCAVNRVLMKLQEQQ
KKNPEMEDLPSKGVQEERLGTEPTSETQDELQRLLGLLRDQDPLSEEQMQGLCIELNDFIV
ALSSVQPSAKREGFVTVPNVTWADIGALEDIREELTMAILAPVRNPDQFKALGLVTPAGVLLA
GPPGCGKTLLAKAVANESGLNFISVKGPELLNMYVGESERAVRQVFQRAKNSAPCVIFFDE
VDALCPRRSDRETGASVRVVNQLLTEMDGLEARQQVFIMAATNRPDIIDPAILRPGRLDKTL
FVGLPPPADRLAILKTITKNGTKPPLDADVNLEAIAGDLRCDCYTGADLSALVREASICALRQ
EMARQKSGNEKGELKVSHKHFEEAFKKVRSSISKKDQIMYERLQESLSRVEHHHHHH
[CD2BP2, Uniprot O95400]
SEQ ID NO: 11
MPKRKVTFQGVGDEEDEDEIIVPKKKLVDPVAGSGGPGSRFKGKHSLDSDEEEDDDDGGS
SKYDILASEDVEGQEAATLPSEGGVRITPFNLQEEMEEGHFDADGNYFLNRDAQIRDSWLD
NIDWVKIRERPPGQRQASDSEEEDSLGQTSMSAQALLEGLLELLLPRETVAGALRRLGARG
GGKGRKGPGQPSSPQRLDRLSGLADQMVARGNLGVYQETRERLAMRLKGLGCQTLGPHN
PTPPPSLDMFAEELAEEELETPTPTQRGEAESRGDGLVDVMWEYKWENTGDAELYGPFTS
AQMQTWVSEGYFPDGVYCRKLDPPGGQFYNSKRIDFDLYT
[CD2BP2, Acc. BC001947]
SEQ ID NO: 12
[CD2BP2, gene synthesis fragment 1, aa1-173]
SEQ ID NO: 13
[CD2BP2, gene synthesis fragment 2, aa174-341]
SEQ ID NO: 14
[CD2BP2, gene synthesis fragment 1, Bbsl]
SEQ ID NO: 15
[CD2BP2, gene synthesis fragment 2, Bbsl]
SEQ ID NO: 16
[sense CD2BP2-fragment-2]
SEQ ID NO: 17
[asense CD2BP2-fragment-2]
SEQ ID NO: 18
[CD2BP2-PCR-fragment]
SEQ ID NO: 19
[pTriEx-1-CD2BP2[human]]
SEQ ID NO: 20
[CD2BP2[human]-H8]
SEQ ID NO: 21
[pET24d-N-CD2BP2[human]]
SEQ ID NO: 22
[H8-CD2BP2[human]]
SEQ ID NO: 23
MSHHHHHHHHSMPKRKVTFQGVGDEEDEDEIIVPKKKLVDPVAGSGGPGSRFKGKHSLDS
DEEEDDDDGGSSKYDILASEDVEGQEAATLPSEGGVRITPFNLQEEMEEGHFDADGNYFLN
RDAQIRDSWLDNIDWVKIRERPPGQRQASDSEEEDSLGQTSMSAQALLEGLLELLLPRETV
AGALRRLGARGGGKGRKGPGQPSSPQRLDRLSGLADQMVARGNLGVYQETRERLAMRLK
GLGCQTLGPHNPTPPPSLDMFAEELAEEELETPTPTQRGEAESRGDGLVDVMWEYKWENT
GDAELYGPFTSAQMQTWVSEGYFPDGVYCRKLDPPGGQFYNSKRIDFDLYT
[TJP1-isoform2, Uniprot Q07157-2]
SEQ ID NO: 24
MSARAAAAKSTAMEETAIWEQHTVTLHRAPGFGFGIAISGGRDNPHFQSGETSIVISDVLKG
GPAEGQLQENDRVAMVNGVSMDNVEHAFAVQQLRKSGKNAKITIRRKKKVQIPVSRPDPE
PVSDNEEDSYDEEIHDPRSGRSGVVNRRSEKIWPRDRSASRERSLSPRSDRRSVASSQPA
KPTKVTLVKSRKNEEYGLRLASHIFVKEISQDSLAARDGNIQEGDVVLKINGTVTENMSLTDA
KTLIERSKGKLKMVVQRDERATLLNVPDLSDSIHSANASERDDISEIQSLASDHSGRSHDRP
PRRSRSRSPDQRSEPSDHSRHSPQQPSNGSLRSRDEERISKPGAVSTPVKHADDHTPKTV
EEVTVERNEKQTPSLPEPKPVYAQVGQPDVDLPVSPSDGVLPNSTHEDGILRPSMKLVKFR
KGDSVGLRLAGGNDVGIFVAGVLEDSPAAKEGLEEGDQILRVNNVDFTNIIREEAVLFLLDLP
KGEEVTILAQKKKDVYRRIVESDVGDSFYIRTHFEYEKESPYGLSFNKGEVFRVVDTLYNGK
LGSWLAIRIGKNHKEVERGIIPNKNRAEQLASVQYTLPKTAGGDRADFWRFRGLRSSKRNLR
KSREDLSAQPVQTKFPAYERVVLREAGFLRPVTIFGPIADVAREKLAREEPDIYQIAKSEPRD
AGTDQRSSGIIRLHTIKQIIDQDKHALLDVTPNAVDRLNYAQWYPIVVFLNPDSKQGVKTMRM
RLCPESRKSARKLYERSHKLRKNNHHLFTTTINLNSMNDGWYGALKEAIQQQQNQLVWVS
EGKADGATSDDLDLHDDRLSYLSAPGSEYSMYSTDSRHTSDYEDTDTEGGAYTDQELDET
LNDEVGTPPESAITRSSEPVREDSSGMHHENQTYPPYSPQAQPQPIHRIDSPGFKPASQQV
YRKDPYPEEMMRQNHVLKQPAVSHPGHRPDKEPNLTYEPQLPYVEKQASRDLEQPTYRYE
SSSYTDQFSRNYEHRLRYEDRVPMYEEQWSYYDDKQPYPSRPPFDNQHSQDLDSRQHPE
ESSERGYFFRFEEPAPLSYDSRPRYEQAPRASALRHEEQPAPGYDTHGRLRPEAQPHPSA
GPKPAESKQYFEQYSRSYEQVPPQGFTSRAGHFEPLHGAAAVPPLIPSSQHKPEALPSNTK
PLPPPPTQTEEEEDPAMKPQSVLTRVKMFENKRSASLETKKDVNDTGSFKPPEVASKPSGA
PIIGPKPTSQNQFSEHDKTLYRIPEPQKPQLKPPEDIVRSNHYDPEEDEEYYRKQLSYFDRR
SFENKPPAHIAASHLSEPAKPAHSQNQSNFSSYSSKGKPPEADGVDRSFGEKRYEPIQATP
PPPPLPSQYAQPSQPVTSASLHIHSKGAHGEGNSVSLDFQNSLVSKPDPPPSQNKPATFRP
PNREDTAQAAFYPQKSFPDKAPVNGTEQTQKTVTPAYNRFTPKPYTSSARPFERKFESPKF
NHNLLPSETAHKPDLSSKTPTSPKTLVKSHSLAQPPEFDSGVETFSIHAEKPKYQINNISTVP
KAIPVSPSAVEEDEDEDGHTVVATARGIFNSNGGVLSSIETGVSIIIPQGAIPEGVEQEIYFKV
CRDNSILPPLDKEKGETLLSPLVMCGPHGLKFLKPVELRLPHCDPKTWQNKCLPGDPNYLV
GANCVSVLIDHF
[sense TJP1-IF2-ZF1]
SEQ ID NO: 25
[asense TJP1-IF2-ZF1]
SEQ ID NO: 26
[sense TJP1-IF2-ZF2]
SEQ ID NO: 27
[asense TJP1-IF2-ZF2]
SEQ ID NO: 28
[TJP1-PCR-fragment-1]
SEQ ID NO: 29
[TJP1-PCR-fragment-2]
SEQ ID NO: 30
[pTriEx-1-TJP1[human]-IF2]
SEQ ID NO: 31
[TJP1[human]-IF2-H8]
SEQ ID NO: 32
[sense NVL-F1]
SEQ ID NO: 33
[asense NVL-F1]
SEQ ID NO: 34
[NVL-PCR-fragment 1-117]
SEQ ID NO: 35
[sense NVL-F2]
SEQ ID NO: 36
[asense NVL-F2]
SEQ ID NO: 37
[NVL-PCR-fragment 106-292]
SEQ ID NO: 38
[sense NVL-F3]
SEQ ID NO: 39
[asense NVL-F3]
SEQ ID NO: 40
[NVL-PCR-fragment 281-560]
SEQ ID NO: 41
[sense NVL-F4]
SEQ ID NO: 42
[asense NVL-F4]
SEQ ID NO: 43
[NVL-PCR-fragment 549-856]
SEQ ID NO: 44
[pET24d-N-NVL[human](aa1-117)]
SEQ ID NO: 45
[pET24d-N-NVL[human](aa106-296)]
SEQ ID NO: 46
[pET24d-N-NVL[human](aa281-560)]
SEQ ID NO: 47
[pET24d-N-NVL[human](aa549-856)]
SEQ ID NO: 48
[H8-NVL[human](aa1-117)]
SEQ ID NO: 49
[H8-NVL[human](aa106-292)]
SEQ ID NO: 50
[H8-NVL[human](aa281-560)]
SEQ ID NO: 51
[H8-NVL[human](aa549-856)]
SEQ ID NO: 52
[sense CD2BP2-F1]
SEQ ID NO: 53
[asense CD2BP2-F1]
SEQ ID NO: 54
[CD2BP2-PCR-fragment 1-128]
SEQ ID NO: 55
[sense CD2BP2-F2]
SEQ ID NO: 56
[asense CD2BP2-F2]
SEQ ID NO: 57
[CD2BP2-PCR-fragment 117-260]
SEQ ID NO: 58
[sense CD2BP2-F3]
SEQ ID NO: 59
[asense CD2BP2-F3]
SEQ ID NO: 60
[CD2BP2-PCR-fragment 249-341]
SEQ ID NO: 61
[pET24d-N-CD2BP2[human](aa1-128)]
SEQ ID NO: 62
[pET24d-N-CD2BP2[human](aa117-260)]
SEQ ID NO: 63
[pET24d-N-CD2BP2[human](aa249-341)]
SEQ ID NO: 64
[H8-CD2BP2[human](aa1-128)]
SEQ ID NO: 65
[H8-CD2BP2[human](aa117-260)]
SEQ ID NO: 66
[H8-CD2BP2[human](aa249-341)]
SEQ ID NO: 67
[sense TJP1-F1/2]
SEQ ID NO: 68
[asense TJP1-F1/4]
SEQ ID NO: 69
[TJP1-PCR-fragment 1-575]
SEQ ID NO: 70
[asense TJP1-F2]
SEQ ID NO: 71
[TJP1-PCR-fragment 1-156]
SEQ ID NO: 72
[sense TJP1-F3]
SEQ ID NO: 73
[asense TJP1-F3]
SEQ ID NO: 74
[TJP1-PCR-fragment 144-306]
SEQ ID NO: 75
[sense TJP1-F4]
SEQ ID NO: 76
[TJP1-PCR-fragment 294-575]
SEQ ID NO: 77
[pET24d-N-TJP1[human](aa1-575)]
SEQ ID NO: 78
[pET24d-N-TJP1[human](aa1-156)]
SEQ ID NO: 79
[pET24d-N-TJP1[human](aa144-306)]
SEQ ID NO: 80
[pET24d-N-TJP1[human](aa294-575)]
SEQ ID NO: 81
[H8-TJP1[humanNaa1-575)]
SEQ ID NO: 82
MSHHHHHHHHSMSARAMAKSTAMEETAIWEQHTVTLHRAPGFGFGIAISGGRDNPHFQS
GETSIVISDVLKGGPAEGQLQENDRVAMVNGVSMDNVEHAFAVQQLRKSGKNAKITIRRKK
KVQIPVSRPDPEPVSDNEEDSYDEEIHDPRSGRSGVVNRRSEKIWPRDRSASRERSLSPRS
DRRSVASSQPAKPTKVTLVKSRKNEEYGLRLASHIFVKEISQDSLAARDGNIQEGDVVLKIN
GTVTENMSLTDAKTLIERSKGKLKMVVQRDERATLLNVPDLSDSIHSANASERDDISEIQSLA
SDHSGRSHDRPPRRSRSRSPDQRSEPSDHSRHSPQQPSNGSLRSRDEERISKPGAVSTP
VKHADDHIPKTVEEVTVERNEKQTPSLPEPKPVYAQVGQPDVDLPVSPSDGVLPNSTHED
GILRPSMKLVKFRKGDSVGLRLAGGNDVGIFVAGVLEDSPAAKEGLEEGDQILRVNNVDFTN
IIREEAVLFLLDLPKGEEVTILAQKKKDVYRRIVESDVGDSFYIRTHFEYEKESPYGLSFNKGE
VFRVVDTLYNGKLGSWLAIRIGKNHKEVERGIIP
[H8-TJP1[human](aa1-156)]
SEQ ID NO: 83
[H8-TJP1[human](aa144-306)]
SEQ ID NO: 84
[H8-TJP1[human](aa294-575)]
SEQ ID NO: 85
[NVL-fragment(aa591-603)]
SEQ ID NO: 86
EELTMAILAPVRN
[NVL-fragment(aa607-617)]
SEQ ID NO: 87
FKALGLVTPAG
[NVL-fragment(aa685-695)]
SEQ ID NO: 88
ALCPRRSDRET
[NVL-fragment(aa742-753)]
SEQ ID NO: 89
KTLFVGLPPPAD
[NVL-fragment(aa761-772)]
SEQ ID NO: 90
ITKNGTKPPLDA
[NVL-fragment(aa778-789)]
SEQ ID NO: 91
AIAGDLRCDCYT
[NVL-fragment(aa809-826)]
SEQ ID NO: 92
MARQKSGNEKGELKVSHK
[NVL-fragment(aa844-856)]
SEQ ID NO: 93
QIMYERLQESLSR

The present invention is further illustrated by the following non-limiting examples from which further features, embodiments, aspects and advantages of the present invention may be taken.

### Example 1

Summary

Methods: Three patient sera (PS1-3) with pre-determined ANA against unknown nuclear autoantigens (UNA), as determined by indirect immunofluorescence assay (IFA) on HEp2 cells and line blot with 23 known nuclear antigens were selected. Sera were analyzed in immunoprecipitation assays with HEp-2 cell lysates and mass spectrometry. The identified candidate autoantigens were recombinantly expressed in E. coli and/or HEK293 cells and were applied in immunoblot, line blot, recombinant IFA and neutralization assays. In addition, the prevalence of ANAs against these antigens in a cohort of patients with systemic autoimmune rheumatic diseases (SARD cohort, n=488) was studied.

Results: Nuclear VCP-like protein (NVL), CD2 antigen cytoplasmic tail-binding protein 2 (CD2BP2), and tight junction protein ZO-1 (TJP1) were identified as the respective target antigen of PS1, PS2 and PS3. In immunoblots with respective purified recombinant antigens NVL, CD2BP2, and TJP1 aa1-575 PS1-3 showed a positive reaction with the individual antigen, whereas healthy controls (n=15) were nonreactive. Preincubation of PS1 with the respective NVL antigen and PS2 with the respective CD2BP2 antigen abolished the ANA reaction of the patient sera in HEp2 cell IFA. Additionally. PS1 and PS3 reacted with the individual antigen in recombinant HEK293 cell IFA. Screening a SARD cohort in a line blot based on the respective purified recombinant antigens NVL, CD2BP2, and TJP1 aa1-575 identified four patients with anti-NVL (4×SSc), ten patients with anti-CD2BP2 (8×SSc, 1×MCTD, 1× Myositis) and 16 patients with anti-TJP1 autoantibodies (15×SSc, 1×UCTD), whereas all control sera (n=50) were negative.

ANAs against NVL, CD2BP2 and TJP1 present novel markers for SARD, especially SSc, or other rheumatic diseases.

Reagents

Reagents were obtained from Merck, Darmstadt, Germany or Sigma-Aldrich, Heidelberg, Germany if not specified otherwise.

Methods

Patients

Control collectives included 50 healthy donor sera. For assessment of the clinical association a cohort of patients with systemic autoimmune rheumatic disease (SARD) was used which consisted of 529 serum samples from 488 patients, of which 378 patients had systemic sclerosis (SSc) and 110 patients had other diseases (anti-synthetase syndrome (ASS) n=10; inclusion body myositis (IBM) n=4; mixed connective tissue disease (MCTD) n=6; myositis n=15; primary Sjögren syndrome (pSS) n=11: rheumatoid arthritis (RA) n=24; systemic lupus erythematosus (SLE) n=30; undifferentiated connective tissue disease (UCTD) n=10). The repeated sera were obtained from the same patients at different time points.

Indirect Immunofluorescence Assay (IFA)

IFA was conducted using slides with a biochip array of acetone fixed HEp-2 cells combined with liver tissue cryosections. Each biochip mosaic was incubated with 35 μL of 1:100 PBS-diluted sample at room temperature for 30 min, washed with PBS-Tween and immersed in PBS-Tween for 5 min. In the second step, anti-human-IgG-Alexa488 (1:500) (Jackson ImmunoResearch, Suffolk, UK) was applied and incubated at a room temperature of 23° C. for 30 min. Slides were washed again with a flush of PBS-Tween and then immersed in PBS-Tween for 5 min. Slides were embedded in PBS-buffered, DABCO containing glycerol (approximately 10 μL per field) and examined by fluorescence microscopy.

For colocalization analysis a mixture of anti-TJP1 antibody (1:50, 33-9100, Thermo Fisher Scientific, Dreieich, Germany) and patient serum (1:10) was used in the first incubation step and anti-human-IgG-alexa488 (1:500) and anti-mouse-IgG-Cy3 (1:200) (Jackson ImmunoResearch) in the second incubation step.

In competitive inhibition experiments, recombinant antigens were mixed with the individual patient serum 1 h prior to the IFA on HEp2 cells. Positive and negative controls were included. Samples were classified as positive or negative based on fluorescence intensity of the transfected cells in direct comparison with control-transfected cells and control samples. Endpoint titers refer to the last dilution showing visible fluorescence.

Results were evaluated by two independent observers using a EUROStar II microscope (EUROIMMUN Medizinische Labordiagnostika AG, Lübeck, Germany).

Immunoprecipitation

The immunoprecipitation was performed with 500 μl cell HEp-2 cells (10×10¹ cells/ml) and 5 μl patient or control sera. HEp-2 cells were centrifuged at 16000×g for 10 minutes at 4° C. The sediment together with patient or control sera was resuspended in 500 μl RIPA lysis buffer (PBS pH 7.4, 0.1% (v/v) Nonidet™ P 40, 1% (w/v) Deoxycholate, 0.1% (w/v) SDS, 1 mM Protease inhibitor cocktail [Sigma]) and rotated for 1 h at 4° C. The suspension was spun down at 16000×g for 20 minutes at 4° C. The supernatants were then incubated with Protein G Dynabeads (ThermoFisher Scientific, Schwerte, Germany) at 4° C. for 3 h to capture immunocomplexes. Beads were washed three times with PBS and eluted with NuPage LDS sample buffer (ThermoFisher Scientific) containing 25 mmol/L dithiothreitol at 70° C. for 10 min. Carbamidomethylation with 59 mM iodoacetamide (Bio-Rad, Hamburg, Germany) was performed prior to SDS-PAGE (NuPAGE, ThermoFisher Scientific). Separated proteins were visualized with Coomassie Brilliant Blue (G-250) and identified by mass spectrometric analysis.

Mass Spectrometry

Visible protein bands were excised from Coomassie Brilliant Blue G-250 stained gels. After destaining and tryptic digestion peptides were extracted and spotted with α-cyano-4-hydroxycinnamic acid onto a MTP AnchorChip™ 384 TF target.

MALDI-TOF/TOF measurements were performed with an Autoflex III smartbeam TOF/TOF200 System using flexControl 3.4 software. MS spectra for peptide mass fingerprinting (PMF) were recorded in positive ion reflector mode with 4,000-10,000 shots and in a mass range from 600 Da to 4,000 Da. Spectra were calibrated externally with the commercially available Peptide Calibration Standard II, processed with flexAnalysis 3.4 and peak lists were analyzed with BioTools 3.2.

The Mascot search engine Mascot Server 2.3 (Matrix Science, London, UK) was used for protein identification by searching against the NCBI or SwissProt database limited to Mammalia. Search parameters were as follows: Mass tolerance was set to 80 ppm, one missed cleavage site was accepted, and carbamidomethylation of cysteine residues as well as oxidation of methionine residues were set as fixed and variable modifications, respectively. To evaluate the protein hits, a significance threshold of p<0.05 was chosen.

For further confirmation of the PMF hits two to five peptides of each identified protein were selected for MS/MS measurements using the WARP feedback mechanism of BioTools. Parent and fragment masses were recorded with 400 and 1000 shots, respectively. Spectra were processed and analyzed as described above with a fragment mass tolerance of 0.7 Da.

Recombinant Expression of Full Length NVL, CD2BP2 and TJP1 in HEK293 or E. coli

NVL-Isoform1 (Nuclear Valosin-Containing Protein-Like)

The cDNA clone IRAUp969F0866D encoding a partial sequence (amino acid 206-856) of human NVL-isoform1 (SEQ ID NO: 1) was obtained from Source BioScience UK Limited. This cDNA clone is coding NVL-isoform3 (Acc. BC012105, SEQ ID NO: 2). A synthetic DNA fragment was ordered coding for the missing amino acids 1-205 of NVL-isoform1 from Eurofins Genomics Germany GmbH (Ebersberg). The gene synthesis product (SEQ ID NO: 3) was digested with BsaI (R3733, New England Biolabs, Frankfurt am Main, Germany) and the 614 bp fragment was isolated by Gel-purification.

The coding sequence for the amino acid 206-856 of NVL-isoform1 was amplified by PCR using cDNA clone IRAUp969F0866D and DNA oligonucleotide primers sense NVL-IF1 (SEQ ID NO: 4) and a sense NVL-IF1 (SEQ ID NO: 5) resulting in SEQ ID NO: 6.

The amplification product was digested with BsaI and together with the gene synthesis fragment ligated with NcoI and XhoI linearized pTriEx-1 vector (Merck, Darmstadt, Germany) resulting in SEQ ID NO: 7, coding for NVL[human]-IF1 fused to an octa Histidin-tag (H8) (SEQ ID NO: 8) or pET24d vector (Merck) resulting in SEQ ID NO: 9, coding for NVL[human]-IF1 fused to a hexa Histidin-tag (H6) (SEQ ID NO: 10).

CD2BP2 (CD2 Antigen Cytoplasmic Tail-Binding Protein 2)

A cDNA clone coding for full length of human CD2BP2 (SEQ ID NO: 11) was not available. Therefore two synthetic gene fragments coding for human CD2BP2 (Acc. B0001947, SEQ ID NO: 12) were obtained from Eurofins Genomics Germany GmbH (Ebersberg).

The first fragment coding for amino acids 1-173 of human CD2BP2 and the other one was coding for amino acids 174-341 of human CD2BP2 including the Stop-codon. Both gene synthesis products (SEQ ID NO: 13 and SEQ ID NO: 14) were digested with BbsI (R3539, New England Biolabs) resulting in 517 bp fragment-1 and 510 bp fragment-2 (SEQ ID NO: 15 and SEQ ID NO: 16) that were both Gel-purified. By PCR a third fragment of CD2BP2 was amplified using the gene synthesis product (SEQ ID NO: 14) as template and the DNA oligonucleotide primers sense CD2BP2-fragment-2 (SEQ ID NO: 17) and asense CD2BP2-fragment-2 (SEQ ID NO: 18), resulting in SEQ ID NO: 19 without the Stop-codon. The amplification product was digested with BbsI (R3539, New England Biolabs).

The fragments SEQ ID NO: 15 and SEQ ID NO: 16 were ligated with NcoI and XhoI linearized pTriEx-1 vector (Merck) resulting in SEQ ID NO: 20, coding for CD2BP2human] fused to a C-terminal octa Histidin-tag (H8) (SEQ ID NO: 21). The fragment SEQ ID NO: 15 and the BbsI-digested fragment SEQ ID NO: 19 were ligated with NcoI and XhoI linearized pET24d vector (Merck) resulting in SEQ ID NO: 22, coding for CD2BP2human] fused to a N-terminal octa Histidin-tag (H8) (SEQ ID NO: 23).

TJP1-Isoform2 (Tight Junction Protein ZO-1)

The cDNA clone IRCMp5012C0531D encoding human TJP1-isoform1 (Acc. BC111712) was obtained from Source BioScience UK Limited. For expression of human TJP1-isoform2 (SEQ ID NO: 24) two Fragments using DNA oligonucleotide primers sense TJP1-IF2-ZF1 (SEQ ID NO: 25) and asense TJP1-IF2-ZF1 (SEQ ID NO: 26) as well as sense TJP1-IF2-ZF2 (SEQ ID NO: 27) and asense TJP1-IF2-ZF2 (SEQ ID NO: 28) were amplified by PCR. One fragment (SEQ ID NO: 29) was digested with BsaI (R3733, New England Biolabs) and the other fragment (SEQ ID NO: 30) was digested with Esp3I (R0734, New England Biolabs). Both fragments were ligated with NcoI and XhoI linearized pTriEx-1 vector (Merck) resulting in SEQ ID NO: 31, coding for human TJP1-isoform2 fused to a C-terminal octa Histidin-tag (H8) (SEQ ID NO: 32).

NVL-His and TJP1-His, respectively, were expressed in the human cell line HEK293 after ExGen500-mediated transfection (ThermoFisher Scientific, Dreieich, Germany) according to the manufacturer's instructions (Biomol GmbH, Hamburg, Germany). For the production of immunofluorescence substrates, the cells were grown on cover slides and acetone fixed two days after transfection. For other purposes cells were harvested 5 d after transfection and lysed with NuPage LDS sample buffer (ThermoFisher Scientific, Schwerte, Germany).

Recombinant Expression of NVL, CD2BP2 and TJP1 Fragments in E. coli

NV

For epitope mapping fragments of human NVL spanning amino acid residues 1 to 117, 106 to 292, 281 to 560 and 549 to 856 were amplified by PCR using SEQ ID NO: 7 as template and the DNA oligonucleotide primers sense NVL-F1 (SEQ ID NO: 33) and asense NVL-F1 (SEQ ID NO: 34) for NVL-fragment 1-117 (SEQ ID NO: 35), sense NVL-F2 (SEQ ID NO: 36) and asense NVL-F2 (SEQ ID NO: 37) for NVL-fragment 106-292 (SEQ ID NO: 38), sense NVL-F3 (SEQ ID NO: 39) and asense NVL-F3 (SEQ ID NO: 40) for NVL-fragment 281-560 (SEQ ID NO: 41) and sense NVL-F4 (SEQ ID NO: 42) and asense NVL-F4 (SEQ ID NO: 43) for NVL-fragment 549-856 (SEQ ID NO: 44). The amplification products were digested with BsaI (R3733, New England Biolabs) and ligated with NcoI and XhoI linearized pET24d vector (Merck) resulting in pET24d-N-NVL[human](aa1-117) (SEQ ID NO: 45), pET24d-N-NVL[human](aa106-296) (SEQ ID NO: 46), pET24d-N-NVL[human](aa281-560) (SEQ ID NO: 47) and pET24d-N-NVL[human](aa549-856) (SEQ ID NO: 48) coding for four proteins fused to a N-terminal octa Histidin-tag (H8) (SEQ ID NO: 49 to SEQ ID NO: 52).

CD2BP2

For epitope mapping fragments of human CD2BP2 spanning amino acid residues 1 to 128, 117 to 260 and 249 to 341 were amplified by PCR using SEQ ID NO: 22 as template and the DNA oligonucleotide primers sense CD2BP2-F1 (SEQ ID NO: 53) and asense CD2BP2-F1 (SEQ ID NO: 54) for CD2BP2-fragment 1-128 (SEQ ID NO: 55), sense CD2BP2-F2 (SEQ ID NO: 56) and asense CD2BP2-F2 (SEQ ID NO: 57) for CD2BP2-fragment 117-260 (SEQ ID NO: 58) and sense CD2BP2-F3 (SEQ ID NO: 59) and asense CD2BP2-F3 (SEQ ID NO: 60) for CD2BP2-fragment 249-341 (SEQ ID NO: 61). The amplification products were digested with Esp3I (R0734, New England Biolabs) and ligated with NcoI and XhoI linearized pET24d vector (Merck) resulting in pET24d-N-CD2BP2[human](aa1-128) (SEQ ID NO: 62), pET24d-N-CD2BP2[human](aa117-260) (SEQ ID NO: 63) and pET24d-N-CD2BP2[human](aa249-341) (SEQ ID NO: 64) coding for three proteins fused to a N-terminal octa Histidin-tag (H8) (SEQ ID NO: 65 to SEQ ID NO: 67).

TJP1

For epitope mapping fragments of human TJP1 spanning amino acid residues 1 to 575, 1 to 156, 144 to 306 and 294 to 575 were amplified by PCR using SEQ ID NO: 31 as template and the DNA oligonucleotide primers sense TJP1-F1/2 (SEQ ID NO: 68) and asense TJP1-F1/4 (SEQ ID NO: 69) for TJP1-fragment 1-575 (SEQ ID NO: 70), sense TJP1-F1/2 (SEQ ID NO: 68) and asense TJP1-F2 (SEQ ID NO: 71) for TJP1-fragment 1-156 (SEQ ID NO: 72), sense TJP1-F3 (SEQ ID NO: 73) and asense TJP1-F3 (SEQ ID NO: 74) for TJP1-fragment 144-306 (SEQ ID NO: 75) and sense TJP1-F4 (SEQ ID NO: 76) and asense TJP1-F1/4 (SEQ ID NO: 69) for TJP1-fragment 294-575 (SEQ ID NO: 77). The amplification products were digested with BsaI (R3733, New England Biolabs) and ligated with NcoI and XhoI linearized pET24d vector (Merck) resulting in pET24d-N-TJP1[human](aa1-575) (SEQ ID NO: 78), pET24d-N-TJP1[human](aa1-156) (SEQ ID NO: 79), pET24d-N-TJP1[human](aa144-306) (SEQ ID NO: 80) and pET24d-N-TJP1[human](aa294-575) (SEQ ID NO: 81) coding for four proteins fused to a N-terminal octa Histidin-tag (H8) (SEQ ID NO: 82 to SEQ ID NO: 85).

Bacterial expression was essentially performed as described in the pET system manual (Merck) employing E. coli strain RosettaBlue(DE3)pLacI (Merck). Protein expression was induced by the addition of 2 mM isopropyl β-D-thiogalactoside (IPTG) for 3 hours at 37° C.

Protein Purification

Bacterial cells were lysed, and proteins were solubilized in 1×PBS pH 7.4, 1 M NaCl, 8 M urea buffer (NVL and TJP1) or 20 mM Tris-HCl pH 8.5, 600 mM NaCl buffer (CD2BP2) and purified by Nib-affinity chromatography and ion exchange chromatography following the protocol as in C. Probst, W. Schlumberger, W. Stocker et al., “Development of ELISA for the specific determination of autoantibodies against envoplakin and periplakin in paraneoplastic pemphigus,” Clinica Chimica Acta, vol. 410, no. 1-2, pp. 13-18, 2009. The proteins were stored in aliquots at −80° C. until further use.

Immunoblot

Purified recombinant antigens (SEQ ID NO: 10, 23, 82) in the respective buffer or recombinant antigen fragments (SEQ ID NO: 49, 50, 51, 52, 66, 67, 83, 84, 85) expressing E. coli or recombinant antigens (SEQ ID NO: 32) containing HEK-cell lysates were incubated with NuPage LDS sample buffer (ThermoFisher Scientific, Schwerte, Germany) containing 25 mmol/L dithiothreitol at 70° C. for 10 min, followed by SDS-PAGE (NuPAGE, ThermoFisher Scientific). Separated proteins were electrotransferred onto a nitrocellulose membrane by tank blotting with transfer buffer (ThermoFisher Scientific) according to the manufacturer's instructions. The membranes were blocked with Universal Blot Buffer plus (EUROIMMUN Medizinische Labordiagnostika AG, Lübeck, Germany) for 15 min and incubated with the patient or control sera (dilution 1:200) or anti-His (1:2000, 70796-3, Merck) in Universal Blot Buffer plus for 3 h, followed by three washing steps with Universal Blot Buffer (EUROIMMUN Medizinische Labordiagnostika AG, Lübeck, Germany), a second incubation for 30 min with anti-human-IgG-AP (1:10, EUROIMMUN Medizinische Labordiagnostika AG, Lübeck, Germany) or anti-mouse-IgG-AP (1:2000, Jackson ImmunoResearch, Suffolk, UK), three washing steps, and staining with NBT/BCIP substrate (EUROIMMUN Medizinische Labordiagnostika AG, Lübeck, Germany). Alternatively, monoclonal anti-His-AP antibody produced in mouse (1:8000, A5588, Sigma-Aldrich), was used for a one-step incubation.

Lineblot

With the Euroline ANA profile (IgG) 23 (DL 1590-5001-23 G, EUROIMMUN Medizinische Labordiagnostika AG, Lübeck, Germany) the 23 most common ANAs were determined (dsDNA, nucleosomes, histones, SS-A, Ro-52, SSB, RNP/Sm, Sm, Mi-2α, Mi-2β, Ku, CENP A, CENP B. Sp100, PML, Scl-70, PM100, PM75, RP11, RP155, gp210, PCNA, DSF70). Briefly, strips were incubated with 1 ml casein sample buffer (EUROIMMUN Medizinische Labordiagnostika AG, Lübeck, Germany) for 5 min and then in 1:101 diluted serum sample for 30 min. Afterwards, each strip was washed three times with 1.5 ml washing buffer for 5 min each time prior to incubation with 1 ml anti-human IgG-AP (1:10, EUROIMMUN Medizinische Labordiagnostika AG, Lubeck, Germany) for 30 min. Strips were washed again as described above and incubated with NBT/BCIP substrate solution (EUROIMMUN Medizinische Labordiagnostika AG, Lübeck, Germany) for 10 min. The reaction was stopped by washing three times with 1 ml distilled water. The strips were analyzed using the software EUROLineScan (EUROIMMUN Medizinische Labordiagnostika AG, Lübeck, Germany).

In addition, recombinant antigens (SEQ ID NO: 10, 23, 82) expressed in E. coli and purified as described above, where diluted in the respective buffer and spotted onto precut (3 mm×5 mm) empty Biodyne™ B membranes (Biodyne™ B Nylon Membrane, 0.45 μm, Thermo Fisher Scientific, Schwerte, Germany). Membranes were dried for 20 min at RT, before patient serum incubation was performed as described above.

Results

Characterization of the Patents' Autoantibodies

The patient sera showed a nucleolar (PS1) or pseudo-dense fine speckled pattern (PS2, PS3) in IFA with HEp2 cells and liver tissue section (FIG. 1). In further monospecific analysis with line blots coated with 23 ANA autoantigens (dsDNA, nucleosomes, histones, SS-A, Ro-52, SSB, RNP/Sm, Sm, Mi-2α, Mi-2β, Ku, CENP A, CENP B, Sp100, PML, Scl-70, PM100, PM75, RP11, RP155, gp210, PCNA, DSF70) the presence of known ANAs was excluded.

Identification of NVL, CD2BP2 and TJP1 as the Target Autoantigens

In order to identify the target antigens patient sera were subjected to immunoprecipitation (IP) with HEp-2 cell homogenate. Immunoprecipitated proteins were resolved by SDS-PAGE and stained with blue silver coomassie stain. In IP eluates of patient sera, bands at approximately 100 kDa (PS 1, band 1), 50 kDa (PS2, band 2) and 220 kDa (PS3, band 3) were detected (FIG. 2), which were absent in control sera IP eluates. The marked bands were analyzed by mass spectrometry and identified as nuclear valosin-containing protein-like (NVL, band 1). CD2 antigen cytoplasmic tail-binding protein 2 (CD2BP2, band 2), or tight junction protein ZO-1 (TJP1, band 3).

Verification of NVL, CD2BP2 and TJP1 as the Target Autoantigens

Human NVL, CD2BP2 and TJP1 aa1-575 were recombinantly expressed in E. coli. The purified antigens were applied in immunoblots and tested with an anti-His-tag antibody, the individual patient serum and healthy control sera (n=15). As it is shown in FIG. 3A-3C, the anti-His-tag antibody and the patient sera showed positive reactions with the recombinant antigens, whereas the fifteen healthy control sera showed no comparable reactivity. A binding of PS3 but not control serum to full length TJP1 could also be detected (FIG. 3D). In addition, IFA with HEK293 cells expressing recombinant NVL or TJP1, confirmed the presence of autoantibodies against these proteins in the respective patient serum (FIG. 4). The purified antigens were further applied in a neutralization test to examine their ability to abolish the IFA ANA pattern of the patient sera. PS1-3 were incubated with the individual antigens or control buffer prior to standard IFA on HEp-2 cells. FIG. 4 shows that preincubation of PS1 with NVL and PS2 with CD2BP2 led to a complete or partial reduction of the ANA pattern on HEp-2 cells. These results confirmed that the respective ANA pattern of PS1 and 2 were indeed caused by an autoantibody targeting NVL or CD2BP2. However, after incubation of PS3 with purified TJP1 aa1-575 the same fluorescence signal was observed compared to the control incubation. This indicates that anti-TJP1 autoantibodies present in PS3 do not cause the observed ANA pattern.

To further confirm this, PS3 and a commercial anti-TJP1 antibody were incubated with HEp-2 cells, recombinant HEK-TJP1 cells and HEK-control cells. As FIG. 6A illustrates, the commercial anti-TJP1 antibody showed a diffused cytoplasmic stain in HEp-2 cells, which was also observed in IFA with PS3. The ANA pattern observed in IFA with PS3 was therefore probably not caused by anti-TJP1 autoantibodies. However, the commercial anti-TJP1 antibody showed the same specific immunoreaction against transfected HEK293 cells expressing TJP1 as PS3 (FIG. 6B) and a specific reaction at junctions between HEK-control cells (FIG. 6C). The magnification of the selected area (FIG. 6D) shows that autoantibodies from PS3 also localized in the area of intercellular contacts, indicating that PS3 recognizes, besides of an unknown nuclear antigen, endogenous TJP1 at cell junctions.

Characterization of the Epitopes Recognized by Anti-NVL, Anti-CD2BP2 and Anti-TJP1 Autoantibodies

In Western Blot with E. coli lysates containing NVL fragments (aa1-117, aa106-292, aa281-560, aa549-856, FIG. 7A), CD2BP2 fragments (aa117-260, aa249-341, FIG. 7B) or TJP1 fragments (aa1-156, aa145-306, aa295-575, FIG. 7C) PS1 or PS2 showed the strongest reactivity with the respective C-terminal fragment of NVL or CD2BP2, indicating that patient's autoantibodies recognize an epitope located in the last 308 aa of NVL or the last 93 aa of CD2BP2. PS3 reacted predominantly with the N-terminal fragment of TJP1, indicating that the epitope is located in the first 156 aa of TJP1.

Further results (data not shown) indicated that one or more epitopes recognized by anti-NVL is or are located on one, two, three, four, five, six, seven or all C-terminal fragments according to SEQ ID NO: 86, SEQ ID NO: 87, SEQ ID NO: 88, SEQ ID NO: 89, SEQ ID NO: 90, SEQ ID NO: 91, SEQ ID NO: 92 and SEQ ID NO: 93.

Validation of the Clinical Association of Anti-NVL, Anti-CD2BP2 and Anti-TJP1 Autoantibodies

For the assessment of the clinical association of anti-NVL, anti-CD2BP2 and anti-TJP1 autoantibodies, a lineblot coated with purified recombinant NVL, CD2BP2 and TJP1 aa1-575 was developed and validated with anti-His-tag-AP, PS1-3 and healthy control sera (n=50) (Tab. 1), before clinical characterized patient collectives were analyzed. PS1-3 showed positive reactions against the corresponding antigens, whereas 50 healthy control sera did not. Following this, 529 sera from 488 patients with systemic rheumatic diseases (SARD cohort) were analyzed. Six sera from four SARD patients were anti-NVL, ten patients were anti-CD2BP2 and 16 patients were anti-TJP1 positive (Tab. 1). Western blot analysis with purified NVL, CD2BP2 or TJP1 aa1-575 confirmed all positive reactions (FIGS. 8A-8C). All anti-NVL positive patients, as well as eight anti-CD2BP2 positive and 15 anti-TJP1 positive patients were diagnosed with SSc. One anti-CD2BP2 positive patient had MCTD and one had myositis. In addition, one anti-TJP1 positive patient suffered on UCTD.

In Tab. 1 summarized data are shown. Line blot with purified NVL, CD2BP2 and TJP1 aa1-575 have been incubated with anti-His-tag-AP, PS1-3, healthy control sera (n=50) and sera from patients with systemic rheumatic diseases (SARD cohort, n=529). Band intensities of all positive reactions and three representative negative control reactions (CS1-3) are shown as well as the diagnosis of anti-NVL, anti-CD2BP2 and anti-TJP1 positive patient sera.

TABLE 1

| | Poly-Ig | | | | | | TJP1 | |
|---|---|---|---|---|---|---|---|---|
| | control | M | G | A | NVL | CD2BP2 | aa1-575 | Diagnosis |
| anti-His-tag | 0 | 6 | 100 | 48 | 15 | 111 | 101 | |
| PS1 | 86 | 0 | 59 | 0 | 107 | 2 | 3 | |
| PS2 | 85 | 0 | 57 | 0 | 1 | 86 | 1 | |
| PS3 | 85 | 0 | 55 | 0 | 3 | 1 | 41 | |
| CS1 | 82 | 0 | 58 | 0 | 4 | 4 | 1 | |
| CS2 | 83 | 0 | 59 | 0 | 0 | 2 | 9 | |
| CS3 | 84 | 0 | 59 | 0 | 1 | 2 | 0 | |
| SARD_34 | 91 | 1 | 31 | 0 | 88 | 5 | 1 | SSc |
| SARD_35 | 91 | 0 | 31 | 0 | 90 | 5 | 1 | |
| SARD_361 | 92 | 0 | 31 | 1 | 105 | 6 | 1 | |
| SARD_37 | 95 | 0 | 36 | 0 | 97 | 3 | 1 | SSc |
| SARD_210 | 84 | 0 | 33 | 0 | 55 | 34 | 1 | SSc |
| SARD_407 | 92 | 0 | 34 | 0 | 87 | 5 | 0 | SSc |
| SARD_66 | 92 | 0 | 34 | 0 | 2 | 50 | 4 | SSc |
| SARD_128 | 88 | 0 | 33 | 0 | 2 | 41 | 2 | SSc |
| SARD_146 | 85 | 0 | 32 | 0 | 2 | 23 | 2 | SSc |
| SARD_210 | 84 | 0 | 33 | 0 | 55 | 34 | 1 | SSc |
| SARD_309 | 92 | 0 | 33 | 0 | 15 | 25 | 1 | SSc |
| SARD_320 | 93 | 0 | 37 | 0 | 2 | 64 | 0 | SSc |
| SARD_399 | 91 | 0 | 35 | 0 | 1 | 49 | 1 | SSc |
| SARD_490 | 95 | 0 | 35 | 0 | 1 | 29 | 1 | SSc |
| SARD_516 | 89 | 0 | 35 | 0 | 6 | 37 | 3 | MCTD |
| SARD_518 | 90 | 0 | 36 | 0 | 2 | 24 | 2 | Myositis |
| SARD_4 | 87 | 0 | 31 | 0 | 13 | 9 | 41 | SSc |
| SARD_19 | 89 | 0 | 32 | 0 | 1 | 2 | 27 | SSc |
| SARD_72 | 87 | 1 | 33 | 0 | 4 | 1 | 23 | SSc |
| SARD_80 | 86 | 0 | 33 | 0 | 2 | 11 | 30 | SSc |
| SARD_89 | 85 | 0 | 32 | 0 | 1 | 2 | 49 | SSc |
| SARD_120 | 82 | 1 | 30 | 0 | 1 | 3 | 27 | SSc |
| SARD_135 | 87 | 0 | 31 | 0 | 4 | 4 | 60 | SSc |
| SARD_153 | 88 | 0 | 32 | 0 | 4 | 3 | 27 | SSc |
| SARD_159 | 87 | 0 | 33 | 0 | 0 | 1 | 60 | SSc |
| SARD_196 | 93 | 0 | 36 | 0 | 7 | 0 | 37 | SSc |
| SARD_324 | 93 | 1 | 34 | 0 | 13 | 7 | 29 | SSc |
| SARD_337 | 88 | 0 | 35 | 0 | 0 | 2 | 28 | SSc |
| SARD_353 | 86 | 0 | 35 | 0 | 3 | 3 | 60 | SSc |
| SARD_372 | 92 | 0 | 36 | 0 | 3 | 1 | 50 | SSc |
| SARD_385 | 91 | 0 | 33 | 0 | 0 | 5 | 26 | SSc |
| SARD_502 | 92 | 0 | 36 | 0 | 7 | 5 | 60 | UCTD |

### Example 2

Supplement: Assessment of Clinical Association of Anti-NVL Autoantibodies with Larger Cohort

The clinical association of anti-NVL autoantibodies was further studied with additional control collectives including healthy donor sera (n=100) and sera from 205 diseased control patients (autoimmune hepatitis (AIH) n=40, rheumatoid arthritis (RA) n=30; systemic lupus erythematosus (SLE) n=135).

For the supplementary study, the additional sera were analyzed with a line blot coated with purified recombinant NVL as described in Example 1. No sera from the additional control collective showed a positive reaction.

Taken data from Example 1 and Example 2 together, only sera from PS1 and six sera of four SSc patients showed positive reaction against recombinant NVL, which were confirmed with Western blot analysis with purified recombinant NVL. No reaction was detected with 150 healthy control sera and 315 diseased control sera.

1. A method, comprising:
detecting, in a sample, an autoantibody binding to a polypeptide having the sequence SEQ ID NO: 1, SEQ ID NO: 11, SEQ ID NO: 24, or a variant thereof.

2. A polypeptide having the sequence SEQ ID NO: 1, SEQ ID NO: 11, SEQ ID NO: 24, or a variant thereof, wherein the polypeptide is immobilized on a carrier.

3. The carrier, comprising the polypeptide according to claim 2, wherein the polypeptide is immobilized on the carrier.

4. An autoantibody, binding to a polypeptide having the sequence SEQ ID NO: 1, SEQ ID NO: 11, SEQ ID NO: 24, or a variant thereof.

5. The autoantibody according to claim 4, wherein the autoantibody is
diluted in a buffer solution,
included in a diluted sample, and/or
in solution with one or more reagents selected from the group consisting of a stabilizer, a washing buffer, and an antidegradant.

6. The method according to claim 1, wherein the sample is from a patient having or suspected of having a systemic autoimmune rheumatic disease or systemic sclerosis.

7. A method for isolating an autoantibody capable of binding to a polypeptide having the sequence SEQ ID NO: 1, SEQ ID NO: 11, SEQ ID NO: 24, or a variant thereof, the method comprising:
a) contacting a sample comprising the autoantibody with the polypeptide immobilized on the carrier according to claim 2, to form a complex of said autoantibody bound to said polypeptide,
b) isolating the complex formed in a),
c) dissociating the complex isolated in b), and
d) separating the autoantibody from the polypeptide, to isolate the autoantibody.

8. A kit, comprising:
the polypeptide according to claim 2, and a means for detecting a complex comprising the polypeptide and an antibody bound to said polypeptide.

9. A kit, comprising:
the carrier according to claim 3, and a means for detecting an autoantibody bound to the carrier or for detecting the polypeptide having the sequence SEQ ID NO: 1, SEQ ID NO: 11, SEQ ID NO: 24, or the variant thereof, or
a diagnostically useful carrier comprising a means for capturing an autoantibody in a liquid solution, and a means for detecting an autoantibody bound to the diagnostically useful carrier.

10. A method, comprising:
manufacturing a kit, medical device, or diagnostic device comprising the carrier according to claim 3.

11. The method according to claim 1, wherein the sample is a bodily fluid comprising antibodies.

12. The method according to claim 1, wherein the autoantibody is detected using a method selected from the group consisting of an immunodiffusion technique, an immunoelectrophoretic technique, a light scattering immunoassay, an agglutination technique, a labeled immunoassay, an enzyme immunoassay, a chemiluminescence immunoassay, and immunofluorescence.

13. A method, comprising:
contacting a medical or diagnostic device comprising a polypeptide having the sequence SEQ ID NO: 1, SEQ ID NO: 11, SEQ ID NO: 24, or a variant thereof, with at least one buffered solution comprising an antibody binding specifically to said polypeptide having the sequence SEQ ID NO: 1, SEQ ID NO: 11, SEQ ID NO: 24, or a variant thereof, wherein the at least one buffered solution is not a sample from a patient in need of a diagnosis where the patient comprises an unknown concentration of said antibody, and
detecting a signal which indicates whether or not the antibody has bound to the polypeptide, and optionally detecting a signal relating to a concentration of the antibody in the at least one buffered solution.

14. A sample, comprising the autoantibody according to claim 4 as a positive control.

15. A diluted sample, comprising the autoantibody according to claim 4, wherein the diluted sample is a sample diluted in an aqueous buffer solution, and/or wherein the diluted sample comprises one or more reagents selected from the group consisting of a calibrator, a stabilizer, a washing buffer, a positive control, and a negative control.

16. The polypeptide according to claim 2, wherein the polypeptide is a purified recombinant protein.

17. The kit according to claim 9, wherein the means for detecting an autoantibody bound to the carrier is a secondary antibody, or wherein the means for capturing an autoantibody in a liquid solution is a non-labeled secondary antibody.

18. The kit according to claim 9, wherein the kit further comprises one or more reagents selected from the group consisting of a calibrator, a washing buffer, a positive control, and a negative control.

19. The method according to claim 11, wherein the bodily fluid comprising antibodies is selected from the group consisting of whole blood, plasma, serum, cerebrospinal fluid, and saliva.

20. The method according to claim 13, wherein
the concentration of the antibody in the at least one buffered solution is known,
the antibody is a recombinant antibody,
the at least one buffered solution is a mixture of two or more solutions comprising the antibody, wherein each of the two or more solutions has a different concentration of the antibody, and/or
the antibody is recognized by secondary antibodies binding specifically to IgG class antibodies.
